SEQUENCE LISTING (224 sequences)

SEQ ID NO: 1 - protein, 293 aa, Artificial Sequence
  Shiga -like toxin 1 Subunit A (SLT- 1A)
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 2 - protein, 293 aa, Artificial Sequence
  Shiga toxin Subunit A (StxA)
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 3 - protein, 297 aa, Artificial Sequence
  Shiga-like toxin 2 Subunit A (SLT-2A)
DEFTVDFSSQKSYVDSLNSIRSAISTPLGNISQGGVSVSVINHVLGGNYISLNVRGLDPYSERFNHLRLIMERNNLYVAGFINTETNIFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 4 - protein, 251 aa, Artificial Sequence
  Shiga toxin subtype c Subunit A (Stx1cA)
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVAR

SEQ ID NO: 5 - protein, 251 aa, Artificial Sequence
  Shiga toxin subtype d Subunit A (Stx1dA)
KEFTLDFSTAKKYVDSLNVIRSAIGTPLQTISSGGTSLLMIDSGTGDNLFAVDIMGLEPEEERFNNLRLIVERNNLYVTGFVNRTNNVFYRFADFSHVTFPGTRAVTLSGDSSYTTLQRVAGISRTGMQINRHSLTTSYLDLMSYSGTSLTQSVARAMLRFVTVTAEALRFRQIQRGFRTTLDDLSGRSYVMTAEDVDLTLNWGRLSSILPDYHGQDSVRVGRISFGSINAILGSVALILNCHHHASRVAR

SEQ ID NO: 6 - protein, 251 aa, Artificial Sequence
  Shiga toxin subtype e Subunit A (Stx1eA)
QDFTVDFSTAKKYVDSLNAIRSAIGTPLHSISSGGTSLLMIDNGTGDNLFAVDIRGLDPEEERFDNLRLIIERNNLYVTGFVNRTSNIFYRFADFSHVTFPGTRAVTLSGDSSYTTLQRVAGIGRTGMQINRHSLTTSYLDLMSYSGSSLTQPVARAMLRFVTVTAEALRFRQIQRGFRTTLDDVSGHSYTMTVEDVDLTLNWGRLSSVLPDYHGQDSVRVGRISFGGVNAILGSVALILNCHHHTSRVSR

SEQ ID NO: 7 - protein, 250 aa, Artificial Sequence
  Shiga toxin subtype 2c Subunit A (Stx2cA) variant 1
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 8 - protein, 250 aa, Artificial Sequence
  Shiga toxin subtype 2c Subunit A (Stx2cA) variant 2
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFAHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 9 - protein, 250 aa, Artificial Sequence
  Shiga toxin subtype 2c Subunit A (Stx2cA) variant 3
REFTIDFSTQQSYVSSLNSIRTEISTPLEHISQGTTSVSVINHTPPGSYFAVDIRGLDIYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 10 - protein, 250 aa, Artificial Sequence
  Shiga toxin subtype 2c Subunit A (Stx2cA) variant 4
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPSVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 11 - protein, 250 aa, Artificial Sequence
  Shiga toxin subtype 2c Subunit A (Stx2cA) variant 5
REFTIDFSTQQSYVSSLNSIRTEISTPLEHISQGTTSVSVINHTPPGSYFAVDIRGLDVYQARFDHLRLIIEQNNLYMAGFVNTATNTFYRFSDFTHISVPSVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 12 - protein, 250 aa, Artificial Sequence
  Shiga toxin subtype 2c Subunit A (Stx2cA) variant 6
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQVLSETAPVYTMTPGDVDLTLNWGRISNVLPEYRGEDGVRVGRISFNNISAILSTVAVILNCHHQGARSVR

SEQ ID NO: 13 - protein, 250 aa, Artificial Sequence
  Shiga toxin subtype 2d Subunit A (Stx2dA) variant 1
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFAHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVIPEYRGEDGVRVGRISFNNISAILGTVAVILNCHHQGARSVR

SEQ ID NO: 14 - protein, 250 aa, Artificial Sequence
  Shiga toxin subtype 2d Subunit A (Stx2dA) variant 2
REFMIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPEEVDLTLNWGRISNVLPEFRGEGGVRVGRISFNNISAILGTVAVILNCHHQGARSVR

SEQ ID NO: 15 - protein, 250 aa, Artificial Sequence
  Shiga toxin subtype 2d Subunit A (Stx2dA) variant 3
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVIPEYRGEDGVRVGRISFNNISAILSTVAVILNCHHQGARSVR

SEQ ID NO: 16 - protein, 250 aa, Artificial Sequence
  Shiga toxin subtype 2e Subunit A (Stx2eA) variant 1
QEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVR

SEQ ID NO: 17 - protein, 250 aa, Artificial Sequence
  Shiga toxin subtype 2e Subunit A (Stx2eA) variant 2
QEFTIDFSTQQSYVSSLNSIRTAISTPLEHISQGATSVSVINHTPPGSYISVGIRGLDVYQAHFDHLRLIIEQNNLYVAGFVNTATNTFYRFSDFAHISLPGVTTISMTTDSSYTTLQRVAALERSGMQISRHSLVSSYLALMEFSGNTMTREASRAVLRFVTVTAEALRFRQIQREFRQALSETAPVYTMTPEDVDLTLNWGRISNVLPEYRGEDGVRVGRISFNNISAILGTVAVILNCHHQGARSVR

SEQ ID NO: 18 - protein, 250 aa, Artificial Sequence
  Shiga toxin subtype 2f Subunit A (Stx2fA)
DEFTVDFSSQKSYVDSLNSIRSAISTPLGNISQGGVSVSVINHVPGGNYISLNVRGLDPYSERFNHLRLIMERNNLYVAGFINTETNTFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVR

SEQ ID NO: 19 - protein, 251 aa, Artificial Sequence
  SLTA-DI-1
AEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAA

SEQ ID NO: 20 - protein, 251 aa, Artificial Sequence
  SLTA-DI-2
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAA

SEQ ID NO: 21 - protein, 251 aa, Artificial Sequence
  SLTA-DI-3
REFTLDFSTARTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLALMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAA

SEQ ID NO: 22 - protein, 501 aa, Artificial Sequence
  HER2 binding molecule 114773
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSA

SEQ ID NO: 23 - protein, 519 aa, Artificial Sequence
  HER2 binding molecule 115172
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSGGGGSGGGGSDIQMTQSPSSLSASVGDRVTITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 24 - protein, 501 aa, Artificial Sequence
  HER2 binding molecule 114778
MAEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSA

SEQ ID NO: 25 - protein, 513 aa, Artificial Sequence
  HER2 binding molecule 114795
MAEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLA

SEQ ID NO: 26 - protein, 499 aa, Artificial Sequence
  HER2 binding molecule 114791
MAEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 27 - protein, 500 aa, Artificial Sequence
  HER2 binding molecule SLTA-DI-1::scFv4
MAEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSA

SEQ ID NO: 28 - protein, 520 aa, Artificial Sequence
  HER2 binding molecule 114912
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 29 - protein, 519 aa, Artificial Sequence
  HER2 binding molecule 115111
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSGGGGSGGGGSDIQMTQSPSSLSASVGDRVTITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 30 - protein, 519 aa, Artificial Sequence
  HER2 binding molecule 115411
MREFTLDFSTARTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLALMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASQVQLVQSGPEVKKPGASVKVSCKASGYPFTNYGMNWVRQAPGQGLEWMGWINTSTGESTFADDFKGRVTMTTDTSTSTTYMELRSLRPDDTAVYFCARWEVYHGYVPYWGQGTLVTVSSGGGGSGGGGSGGGGSGGGGSGGGGSDIQMTQSPSSLSASIGDRVTITCKASQDVYNAVAWYQQKPGEAPKLLVYSASSRYTGVPSRFSGSGSGTDFTFTISSLQPEDIATYFCQQHFRTPFTFAPGTKLEIK

SEQ ID NO: 31 - protein, 386 aa, Artificial Sequence
  HER2 binding molecule 114898
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAAHHSEDPSSKAPKAPEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSA

SEQ ID NO: 32 - protein, 499 aa, Artificial Sequence
  HER2 binding molecule 115195
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSDIQMTQSPSSLSASVGDRVTITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 33 - protein, 499 aa, Artificial Sequence
  HER2 binding molecule 115194
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSDIQMTQSPSSLSASVGDRVTITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 34 - protein, 371 aa, Artificial Sequence
  HER2 binding molecule 115645
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSA

SEQ ID NO: 35 - protein, 373 aa, Artificial Sequence
  HER2 binding molecule 115845
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLALMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSSHHHHHH

SEQ ID NO: 36 - protein, 514 aa, Artificial Sequence
  SLTA-DI-2::scFv8
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSGGGGSDIQMTQSPSSLSASVGDRVTITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 37 - protein, 251 aa, Artificial Sequence
  STLA-FR
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 38-44 - skipped (7 entries)

SEQ ID NO: 45 - protein, 5 aa, Artificial Sequence
  vhCDR1
DTYIH

SEQ ID NO: 46 - protein, 17 aa, Artificial Sequence
  vhCDR2
RIYPTNGYTRYADSVKG

SEQ ID NO: 47 - protein, 11 aa, Artificial Sequence
  vhCDR3
WGGDGFYAMDY

SEQ ID NO: 48 - protein, 11 aa, Artificial Sequence
  vlCDR1
RASQDVNTAVA

SEQ ID NO: 49 - protein, 7 aa, Artificial Sequence
  vlCDR2
SASFLYS

SEQ ID NO: 50 - protein, 9 aa, Artificial Sequence
  vlCDR3
QQHYTTPPT

SEQ ID NO: 51 - protein, 5 aa, Artificial Sequence
  vhCDR1
SYWIA

SEQ ID NO: 52 - protein, 17 aa, Artificial Sequence
  vhCDR2
LIYPGDSDTKYSPSFQG

SEQ ID NO: 53 - protein, 20 aa, Artificial Sequence
  vhCDR3
HDVGYCSSSNCAKWPEYFQH

SEQ ID NO: 54 - protein, 13 aa, Artificial Sequence
  vlCDR1
SGSSSNIGNNYVS

SEQ ID NO: 55 - protein, 7 aa, Artificial Sequence
  vlCDR2
SASYRYT

SEQ ID NO: 56 - protein, 9 aa, Artificial Sequence
  vlCDR3
QQYYIYPYT

SEQ ID NO: 57 - protein, 5 aa, Artificial Sequence
  vhCDR1
NYGMN

SEQ ID NO: 58 - protein, 17 aa, Artificial Sequence
  vhCDR2
WINTSTGESTFADDFKG

SEQ ID NO: 59 - protein, 10 aa, Artificial Sequence
  vhCDR3
WEVYHGYVPY

SEQ ID NO: 60 - protein, 11 aa, Artificial Sequence
  vlCDR1
KASQDVYNAVA

SEQ ID NO: 61 - protein, 7 aa, Artificial Sequence
  vlCDR2
SASSRYT

SEQ ID NO: 62 - protein, 9 aa, Artificial Sequence
  vlCDR3
QQHFRTPFT

SEQ ID NO: 63 - protein, 5 aa, Artificial Sequence
  vhCDR1
DYTMD

SEQ ID NO: 64 - protein, 17 aa, Artificial Sequence
  vhCDR2
DVNPNSGGSIYNQRFKG

SEQ ID NO: 65 - protein, 10 aa, Artificial Sequence
  vhCDR3
NLGPSFYFDY

SEQ ID NO: 66 - protein, 11 aa, Artificial Sequence
  vlCDR1
KASQDVSIGVA

SEQ ID NO: 67 - protein, 7 aa, Artificial Sequence
  vlCDR2
SASYRYT

SEQ ID NO: 68 - protein, 9 aa, Artificial Sequence
  vlCDR3
QQYYIYPYT

SEQ ID NO: 69 - protein, 5 aa, Artificial Sequence
  vhCDR1
INTMG

SEQ ID NO: 70 - protein, 16 aa, Artificial Sequence
  vhCDR2
LISSIGDTYYADSVKG

SEQ ID NO: 71 - protein, 10 aa, Artificial Sequence
  vhCDR3
FRTAAQGTDY

SEQ ID NO: 72 - protein, 5 aa, Artificial Sequence
  vlCDR1
SCGMG

SEQ ID NO: 73 - protein, 16 aa, Artificial Sequence
  vlCDR2
RLSGDGDTWHKESVKG

SEQ ID NO: 74 - protein, 7 aa, Artificial Sequence
  vlCDR3
CYNLETY

SEQ ID NO: 75 - protein, 251 aa, Artificial Sequence
  SLT-1A-combo variant 1
KEFILRFSVAHKYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 76 - protein, 251 aa, Artificial Sequence
  SLT-1A-combo variant 2
KEFTLDFSTAKTYVDSLNVIRSAIGTPLQTISSGGTSLLMIDNLVPMVATVVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 77 - protein, 251 aa, Artificial Sequence
  SLT-1A-combo variant 3
KEFTLDFSTAKTYVDSLNVIRSAIGTPLQTISSGGTSLLMIDSNLVPMVATVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 78 - protein, 251 aa, Artificial Sequence
  SLT-1A-combo variant 4
KEFTLDFSTAKTYVDSLNVIRSAIGTPLQTISSGGTSLLMIDSGILGFVFTLDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 79 - protein, 251 aa, Artificial Sequence
  SLT-1A-combo variant 5
KEFTLDFSTAKTYVDSLNVIRSAIGTPLQTISSGGTSLLMIDSGSGDNLFAVGILGFDFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 80 - protein, 251 aa, Artificial Sequence
  SLT-1A-combo variant 6
KEFTLDFSTAKTYVDSLNVIRSAIGTPLQTISSGGTSLLMIDSGSGDNLFAVDILGFVFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 81 - protein, 251 aa, Artificial Sequence
  SLT-1A-combo variant 7
KEFTLDFSTAKTYVDSLNVIRSAIGTPLQTISSGGTSLLMIDSGSGDNLFAVDILGFDFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 82 - protein, 251 aa, Artificial Sequence
  SLT-1A-combo variant 8
KEFTLDFSTAKTYVDSLNVIRSAIGTPLQTISSGGTSLLMIDSGSGDNLFAVGILGFVFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 83 - protein, 251 aa, Artificial Sequence
  SLT-1A-combo variant 9
KEFILDFSTAKTYVDSLNVIRSAIGTPLQTISSGGTSLLMIDSGIGDNLFAVDVRGIAPIEARFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLAALSGASYVMTAEDVDLTLNWGRLSSVLPDYHGQDSVRVGRISFGSINAILGSVALILNSHHHASAVAA

SEQ ID NO: 84 - protein, 252 aa, Artificial Sequence
  SLT-1A-combo variant 10
KEFTLDFSTAKTYVDSLNVIRSAIGTPLQTISSGGTSLLMIDSGSGDNLFAVGILGFVFTLEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 85 - protein, 251 aa, Artificial Sequence
  SLT-1A-combo variant 11
KEFTLDFSTAKTYVDSLNVIRSAIGTPLQTISSGGTSLLMIDSGSGDNLFAVNLVPMVATVGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 86 - protein, 251 aa, Artificial Sequence
  SLT-1A-combo variant 12
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTNLVPMVATVSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 87 - protein, 251 aa, Artificial Sequence
  SLT-1A-combo variant 13
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRGILGDVFTLSYVMTAEDVDLTLNWGRLSSVLPDYHGQDSVRVGRISFGSINAILGSVALILNSHHHASAVAA

SEQ ID NO: 88 - protein, 260 aa, Artificial Sequence
  SLT-1A-combo variant 14
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHILRFSVAHKASAVAA

SEQ ID NO: 89 - protein, 262 aa, Artificial Sequence
  SLT-1A-combo variant 15
KEFTLDFSTAKTYVDSLNVIRSAIGTPLQTISSGGTSLLMIDSGSGDNLFAVDILGFDFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHARNLVPMVATVASAVAA

SEQ ID NO: 90-96 - skipped (7 entries)

SEQ ID NO: 97 - protein, 500 aa, Artificial Sequence
  HER2-targeting molecule 1
AEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSA

SEQ ID NO: 98 - protein, 512 aa, Artificial Sequence
  HER2-targeting molecule 2
AEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLA

SEQ ID NO: 99 - protein, 498 aa, Artificial Sequence
  HER2-targeting molecule 3
AEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 100 - protein, 499 aa, Artificial Sequence
  HER2-targeting molecule 4
AEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSA

SEQ ID NO: 101 - protein, 519 aa, Artificial Sequence
  HER2-targeting molecule 5
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 102 - protein, 518 aa, Artificial Sequence
  HER2-targeting molecule 6
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSGGGGSGGGGSDIQMTQSPSSLSASVGDRVTITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 103 - protein, 518 aa, Artificial Sequence
  HER2-targeting molecule 7
REFTLDFSTARTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLALMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASQVQLVQSGPEVKKPGASVKVSCKASGYPFTNYGMNWVRQAPGQGLEWMGWINTSTGESTFADDFKGRVTMTTDTSTSTTYMELRSLRPDDTAVYFCARWEVYHGYVPYWGQGTLVTVSSGGGGSGGGGSGGGGSGGGGSGGGGSDIQMTQSPSSLSASIGDRVTITCKASQDVYNAVAWYQQKPGEAPKLLVYSASSRYTGVPSRFSGSGSGTDFTFTISSLQPEDIATYFCQQHFRTPFTFAPGTKLEIK

SEQ ID NO: 104 - protein, 385 aa, Artificial Sequence
  HER2-targeting molecule 8
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAAHHSEDPSSKAPKAPEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSA

SEQ ID NO: 105 - protein, 498 aa, Artificial Sequence
  HER2-targeting molecule 9
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSDIQMTQSPSSLSASVGDRVTITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 106 - protein, 370 aa, Artificial Sequence
  HER2-targeting molecule 10
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSA

SEQ ID NO: 107 - protein, 366 aa, Artificial Sequence
  HER2-targeting molecule 11
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLALMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 108 - protein, 513 aa, Artificial Sequence
  HER2-targeting molecule 12
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSGGGGSDIQMTQSPSSLSASVGDRVTITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 109-186 - skipped (78 entries)

SEQ ID NO: 187 - protein, 8 aa, Artificial Sequence
  linker sequence
  SITE (3)..(4): may be absent
  SITE (5)..(6): may be absent
  SITE (7)..(8): may be absent
GSGSGSGS

SEQ ID NO: 188 - protein, 20 aa, Artificial Sequence
  linker sequence
  SITE (6)..(10): may be absent
  SITE (11)..(15): may be absent
  SITE (16)..(20): may be absent
GSGGSGSGGSGSGGSGSGGS

SEQ ID NO: 189 - protein, 20 aa, Artificial Sequence
  linker sequence
  SITE (6)..(10): may be absent
  SITE (11)..(15): may be absent
  SITE (16)..(20): may be absent
GGGGSGGGGSGGGGSGGGGS

SEQ ID NO: 190 - protein, 16 aa, Artificial Sequence
  linker sequence
  SITE (5)..(8): may be absent
  SITE (9)..(12): may be absent
  SITE (13)..(16): may be absent
GGGSGGGSGGGSGGGS

SEQ ID NO: 191-194 - skipped (4 entries)

SEQ ID NO: 195 - protein, 4 aa, Artificial Sequence
  Motif
  SITE (2)..(3): Xaa is any amino acid
RXXR

SEQ ID NO: 196-198 - skipped (3 entries)

SEQ ID NO: 199 - protein, 27 aa, Artificial Sequence
  Linker
SSGGGGSGGGGSGGGGSGGGGSGGGGS

SEQ ID NO: 200 - protein, 5 aa, Artificial Sequence
  Linker
GSGSG

SEQ ID NO: 201 - protein, 6 aa, Artificial Sequence
  Linker
ASGGPE

SEQ ID NO: 202 - protein, 54 aa, Artificial Sequence
  linker sequence
  SITE (5)..(6): Up to 2 residues may be absent
  SITE (7)..(12): May be absent
  SITE (10)..(11): Up to 2 residues may be absent
  SITE (13)..(17): May be absent
  SITE (15)..(16): Up to 2 residues may be absent
  SITE (18)..(22): May be absent
  SITE (20)..(21): Up to 2 residues may be absent
  SITE (23)..(27): May be absent
  SITE (25)..(26): Up to 2 residues may be absent
  SITE (28)..(32): May be absent
  SITE (30)..(31): Up to 2 residues may be absent
  SITE (33)..(37): May be absent
  SITE (35)..(36): Up to 2 residues may be absent
  SITE (38)..(42): May be absent
  SITE (40)..(41): Up to 2 residues may be absent
  SITE (43)..(47): May be absent
  SITE (45)..(46): Up to 2 residues may be absent
  SITE (48)..(52): May be absent
  SITE (50)..(51): Up to 2 residues may be absent
AMGGGGSGGGGSGGGGSGGGGSGGGGSGGGGSGGGGSGGGGSGGGGSGGGGSAM

SEQ ID NO: 203 - protein, 210 aa, Artificial Sequence
  linker sequence
  SITE (2)..(6): Up to 5 residues may be absent
  SITE (8)..(14): May be absent
  SITE (9)..(13): Up to 5 residues may be absent
  SITE (15)..(21): May be absent
  SITE (16)..(20): Up to 5 residues may be absent
  SITE (22)..(28): May be absent
  SITE (23)..(27): Up to 5 residues may be absent
  SITE (29)..(35): May be absent
  SITE (30)..(34): Up to 5 residues may be absent
  SITE (36)..(42): May be absent
  SITE (37)..(41): Up to 5 residues may be absent
  SITE (42)..(48): Up to 5 residues may be absent
  SITE (43)..(49): May be absent
  SITE (50)..(56): May be absent
  SITE (51)..(55): Up to 5 residues may be absent
  SITE (57)..(63): May be absent
  SITE (58)..(61): Up to 5 residues may be absent
  SITE (64)..(70): May be absent
  SITE (65)..(69): Up to 5 residues may be absent
  SITE (71)..(77): May be absent
  SITE (72)..(76): Up to 5 residues may be absent
  SITE (78)..(84): May be absent
  SITE (79)..(83): Up to 5 residues may be absent
  SITE (85)..(91): May be absent
  SITE (86)..(90): Up to 5 residues may be absent
  SITE (92)..(98): May be absent
  SITE (93)..(97): Up to 5 residues may be absent
  SITE (99)..(105): May be absent
  SITE (100)..(104): Up to 5 residues may be absent
  SITE (106)..(112): May be absent
  SITE (107)..(111): Up to 5 residues may be absent
  SITE (113)..(119): May be absent
  SITE (114)..(118): Up to 5 residues may be absent
  SITE (120)..(126): May be absent
  SITE (121)..(125): Up to 5 residues may be absent
  SITE (127)..(133): May be absent
  SITE (128)..(132): Up to 5 residues may be absent
  SITE (134)..(140): May be absent
  SITE (135)..(139): Up to 5 residues may be absent
  SITE (141)..(147): May be absent
  SITE (142)..(146): Up to 5 residues may be absent
  SITE (148)..(154): May be absent
  SITE (149)..(153): Up to 5 residues may be absent
  SITE (155)..(161): May be absent
  SITE (156)..(160): Up to 5 residues may be absent
  SITE (162)..(168): May be absent
  SITE (163)..(167): Up to 5 residues may be absent
  SITE (169)..(175): May be absent
  SITE (170)..(174): Up to 5 residues may be absent
  SITE (176)..(182): May be absent
  SITE (177)..(181): Up to 5 residues may be absent
  SITE (183)..(189): May be absent
  SITE (184)..(188): Up to 5 residues may be absent
  SITE (190)..(196): May be absent
  SITE (191)..(195): Up to 5 residues may be absent
  SITE (197)..(203): May be absent
  SITE (198)..(202): Up to 5 residues may be absent
  SITE (204)..(210): May be absent
  SITE (205)..(209): Up to 5 residues may be absent
GGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGS

SEQ ID NO: 204 - protein, 210 aa, Artificial Sequence
  linker sequence
  SITE (2)..(6): Up to 5 residues may be absent
  SITE (8)..(14): May be absent
  SITE (9)..(13): Up to 5 residues may be absent
  SITE (15)..(21): May be absent
  SITE (16)..(20): Up to 5 residues may be absent
  SITE (22)..(28): May be absent
  SITE (23)..(27): Up to 5 residues may be absent
  SITE (29)..(35): May be absent
  SITE (30)..(34): Up to 5 residues may be absent
  SITE (36)..(42): May be absent
  SITE (37)..(41): Up to 5 residues may be absent
  SITE (42)..(48): Up to 5 residues may be absent
  SITE (43)..(49): May be absent
  SITE (50)..(56): May be absent
  SITE (51)..(55): Up to 5 residues may be absent
  SITE (57)..(63): May be absent
  SITE (58)..(61): Up to 5 residues may be absent
  SITE (64)..(70): May be absent
  SITE (65)..(69): Up to 5 residues may be absent
  SITE (71)..(77): May be absent
  SITE (72)..(76): Up to 5 residues may be absent
  SITE (78)..(84): May be absent
  SITE (79)..(83): Up to 5 residues may be absent
  SITE (85)..(91): May be absent
  SITE (86)..(90): Up to 5 residues may be absent
  SITE (92)..(98): May be absent
  SITE (93)..(97): Up to 5 residues may be absent
  SITE (99)..(105): May be absent
  SITE (100)..(104): Up to 5 residues may be absent
  SITE (106)..(112): May be absent
  SITE (107)..(111): Up to 5 residues may be absent
  SITE (113)..(119): May be absent
  SITE (114)..(118): Up to 5 residues may be absent
  SITE (120)..(126): May be absent
  SITE (121)..(125): Up to 5 residues may be absent
  SITE (127)..(133): May be absent
  SITE (128)..(132): Up to 5 residues may be absent
  SITE (134)..(140): May be absent
  SITE (135)..(139): Up to 5 residues may be absent
  SITE (141)..(147): May be absent
  SITE (142)..(146): Up to 5 residues may be absent
  SITE (148)..(154): May be absent
  SITE (149)..(153): Up to 5 residues may be absent
  SITE (155)..(161): May be absent
  SITE (156)..(160): Up to 5 residues may be absent
  SITE (162)..(168): May be absent
  SITE (163)..(167): Up to 5 residues may be absent
  SITE (169)..(175): May be absent
  SITE (170)..(174): Up to 5 residues may be absent
  SITE (176)..(182): May be absent
  SITE (177)..(181): Up to 5 residues may be absent
  SITE (183)..(189): May be absent
  SITE (184)..(188): Up to 5 residues may be absent
  SITE (190)..(196): May be absent
  SITE (191)..(195): Up to 5 residues may be absent
  SITE (197)..(203): May be absent
  SITE (198)..(202): Up to 5 residues may be absent
  SITE (204)..(210): May be absent
  SITE (205)..(209): Up to 5 residues may be absent
SSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSG

SEQ ID NO: 205 - protein, 150 aa, Artificial Sequence
  linker sequence
  SITE (6)..(10): May be absent
  SITE (11)..(15): May be absent
  SITE (16)..(20): May be absent
  SITE (21)..(25): May be absent
  SITE (26)..(30): May be absent
  SITE (31)..(35): May be absent
  SITE (36)..(40): May be absent
  SITE (41)..(45): May be absent
  SITE (46)..(50): May be absent
  SITE (51)..(55): May be absent
  SITE (56)..(60): May be absent
  SITE (61)..(65): May be absent
  SITE (66)..(70): May be absent
  SITE (71)..(75): May be absent
  SITE (76)..(80): May be absent
  SITE (81)..(85): May be absent
  SITE (86)..(90): May be absent
  SITE (91)..(95): May be absent
  SITE (96)..(100): May be absent
  SITE (101)..(105): May be absent
  SITE (106)..(110): May be absent
  SITE (111)..(115): May be absent
  SITE (116)..(120): May be absent
  SITE (121)..(125): May be absent
  SITE (126)..(130): May be absent
  SITE (131)..(135): May be absent
  SITE (136)..(140): May be absent
  SITE (141)..(145): May be absent
  SITE (146)..(150): May be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 206 - protein, 30 aa, Artificial Sequence
  linker sequence
  SITE (2)..(30): Up to 29 may be absent
GGGGGGGGGGGGGGGGGGGGGGGGGGGGGG

SEQ ID NO: 207 - protein, 12 aa, Artificial Sequence
  Linker
GKSSGSGSESKS

SEQ ID NO: 208 - protein, 14 aa, Artificial Sequence
  Linker
EGKSSGSGSESKEF

SEQ ID NO: 209 - protein, 14 aa, Artificial Sequence
  Linker
GSTSGSGKSSEGKG

SEQ ID NO: 210 - protein, 18 aa, Artificial Sequence
  Linker
GSTSGSGKSSEGSGSTKG

SEQ ID NO: 211 - protein, 18 aa, Artificial Sequence
  Linker
GSTSGSGKPGSGEGSTKG

SEQ ID NO: 212 - protein, 5 aa, Artificial Sequence
  Linker
SRSSG

SEQ ID NO: 213 - protein, 5 aa, Artificial Sequence
  Linker
SGSSC

SEQ ID NO: 214 - protein, 25 aa, Artificial Sequence
  linker 1
GGGGSGGGGSGGGGSGGGGSGGGGS

SEQ ID NO: 215 - protein, 20 aa, Artificial Sequence
  linker 2
GGGGSGGGGSGGGGSGGGGS

SEQ ID NO: 216 - protein, 18 aa, Artificial Sequence
  linker 3
GSTSGSGKPGSGEGSTKG

SEQ ID NO: 217 - protein, 5 aa, Artificial Sequence
  linker 4
GGGGS

SEQ ID NO: 218 - protein, 16 aa, Artificial Sequence
  linker 5
EFPKPSTPPGSSGGAP

SEQ ID NO: 219 - protein, 25 aa, Artificial Sequence
  linker 6
EFPKPSTPPGSSGGAPGILGFVFTL

SEQ ID NO: 220 - protein, 15 aa, Artificial Sequence
  linker 7
GSTSGSGKPGSGEGS

SEQ ID NO: 221 - protein, 16 aa, Artificial Sequence
  linker 8
SPSTPPTPSPSTPPAS

SEQ ID NO: 222 - protein, 15 aa, Artificial Sequence
  linker 9
AHHSEDPSSKAPKAP

SEQ ID NO: 223 - protein, 28 aa, Artificial Sequence
  Protease
AMGRSGGGCAGNRVGSSLSCGGLNLQAM

SEQ ID NO: 224 - protein, 251 aa, Artificial Sequence
  HER2 binding region
QVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSGGGGSGGGGSDIQMTQSPSSLSASVGDRVTITCKASQDVYNAVAWYQQKPGQSPKLLIYSASSRYTGVPSRFTGSGSGPDFTFTISSVQAEDLAVYFCQQHFRTPFTFGSGTKLEIK

(19) United States
(12) Patent Application Publication
(10) Pub. No.: US 2023/0295342 A1
(43) Pub. Date: Sep. 21, 2023

(54) CLINICAL METHODS FOR USE OF HER2 BINDING MOLECULES

(71) Applicants: Eric POMA, New York, NY (US); Roger WALTZMAN, New York, NY (US); Jack HIGGINS, Georgetown, TX (US); Eric WILLIAMS, Leander, TX (US); Molecular Templates, Inc., Austin, TX (US)

(72) Inventors: Eric POMA, New York, NY (US); Roger WALTZMAN, New York, NY (US); Jack HIGGINS, Georgetown, TX (US); Eric WILLIAMS, Leander, TX (US)

(21) Appl. No.: 18/006,216

(22) PCT Filed: Jul. 22, 2021
(86) PCT No.: PCT/US2021/042772

§ 371 Date: Jan. 20, 2023

Related U.S. Application Data
(60) Provisional application No. 63/055,074, filed on Jul. 22, 2020.

(51) Int. Cl. C07K 16/32; A61K 47/68; A61P 35/00

(52) U.S. Cl. CPC C07K 16/32; A61K 47/6829; A61P 35/00; A61K 2039/505

(57) ABSTRACT

Provided herein are methods for treating or preventing cancer comprising administering to a subject in need thereof an effective amount of a HER2 binding molecule comprising a cytotoxic Shiga toxin A subunit effector polypeptide and a binding region capable of specifically binding an extracellular part of human HER2. The cancer may be a cancer that involves a cell which expresses or overexpresses HER2, such as a HER2-positive breast cancer, bile duct cancer, or a gastric or gastroesophageal adenocarcinoma.

### CROSS-REFERENCE TO RELATED APPLICATIONS

This application claims priority to U.S. Provisional Application No. 63/055,074, filed on Jul. 22, 2020, which is hereby incorporated by reference in its entirety.

### FIELD

The instant application relates to compositions and methods for treating cancer. More specifically, the instant application relates to the use of HER2 targeting molecules comprising Shiga toxin effector peptides to selectively target cancer cells.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is MTEM_015_01WO_Seq_List_ST25.txt. The file is approximately 229 KB, was created on Jul. 22, 2021, and is being submitted electronically.

### BACKGROUND

HER2 (human epidermal growth factor receptor 2) is a type I transmembrane tyrosine kinase receptor of the ErbB family. HER2 is an attractive molecular target for therapeutics because of its overexpression on the surfaces of cancer cells, its correlation with poor prognoses, and its functional roles in tumorigenesis and cancer development, such as invasiveness and metastasis, and anti-neoplastic drug resistance. HER2 is prominently associated with the pathogenesis, progression and prognosis of certain breast cancers, and other cancers such as colorectal, endometrial, esophageal, gastric, head and neck, lung, ovarian, prostate, pancreatic, and testicular germ cell cancers. Notably, overexpression of HER2 in a tumor cell can confer drug resistance to anti-neoplastic agents.

HER2-targeted therapies have improved outcomes in HER2-positive cancers. However, there are challenges with currently available HER2-targeted therapies. Despite the progress in outcomes for HER2-positive cancers with targeted therapies and the increased availability of treatment options over time, many patients still relapse and need additional therapies to control their disease. Additionally, resistance to known therapies such as trastuzumab, pertuzumab, ado-trastuzumab emtansine (T-DM1), and HER2 tyrosine kinase inhibitors has been reported, and may develop over time, requiring additional therapeutic options. Many tumors retain HER2 expression and may respond to combination therapies that target different domains of HER2, allowing for a synergistic effect and prolonged stable disease.

Accordingly, there remains a need in the art for improved HER2-targeted therapies, and clinical methods for use thereof.

### BRIEF SUMMARY

Provided herein are methods for treating or preventing cancer comprising administering to a subject in need thereof an effective amount of a HER2 binding molecule comprising: a cytotoxic Shiga toxin A subunit effector polypeptide and a binding region capable of specifically binding an extracellular part of human HER2. In some embodiments, the binding region comprises: (a) an immunoglobulin heavy chain variable region comprising: a CDR1 comprising the sequence of SEQ ID NO: 57; a CDR2 comprising the sequence of SEQ ID NO: 58; and a CDR3 comprising the sequence of SEQ ID NO: 59; and (b) an immunoglobulin light chain variable region comprising: a CDR1 comprising the sequence of SEQ ID NO: 60; a CDR2 comprising the sequence of SEQ ID NO: 61; and a CDR3 comprising the sequence of SEQ ID NO: 62. In some embodiments, the effective amount is a dose in the range of about 0.1 to about 50 μg/kg.

In some embodiments, the dose is about 0.5 μg/kg, about 1.0 μg/kg, about 2.0 μg/kg, about 3.0 μg/kg, about 4.5 μg/kg, about 6.75 μg/kg, about 10.0 μg/kg, about 12.5 μg/kg, about 15.0 μg/kg, about 15.6 μg/kg, about 19.5 μg/kg, about 22.5 μg/kg, or about 33.75 μg/kg. In some embodiments, the dose is in the range of about 12.5 μg/kg to about 15 μg/kg, about 15.6 μg/kg to about 22.5 μg/kg, or about 19.5 μg/kg to about 33.75 μg/kg.

In some embodiments, the HER2 binding molecule is administered to the subject by intravenous, subcutaneous, or intramuscular injection. In some embodiments, the HER2 binding molecule is administered to the subject by intravenous injection.

In some embodiments, the HER2 binding molecule is administered to the subject over a period of about 10 minutes to about 1 hour. In some embodiments, the HER2 binding molecule is administered to the subject over a period of about 30 minutes.

In some embodiments, the HER2 binding molecule is administered to the subject once. In some embodiments, the HER2 binding molecule is administered to the subject more than once. In some embodiments, the HER2 binding molecule is administered to the subject every seven days. In some embodiments, the HER2 binding molecule is administered to the subject over a 21 day cycle.

In some embodiments, the subject is administered a dose in the range of about 0.1 μg/kg to about 50 μg/kg at each administration. In some embodiments, the subject is administered a dose of about 0.5 μg/kg, about 1.0 μg/kg, about 2.0 μg/kg, about 3.0 μg/kg, about 4.5 μg/kg, about 6.75 μg/kg, about 10.0 μg/kg, about 12.5 μg/kg, about 15.0 μg/kg, about 15.6 μg/kg, about 19.5 μg/kg, about 22.5 μg/kg, or about 33.75 μg/kg at each administration.

In some embodiments, the HER2 binding molecule is administered to the subject on days 1, 8, and 15 of the 21 day cycle. In some embodiments, the method comprises administering to the subject 0.5 μg/kg of the HER2 binding molecule on days 1, 8, and 15. In some embodiments, the method comprises administering to the subject 1.0 μg/kg of the HER2 binding molecule on days 1, 8, and 15. In some embodiments, the method comprises administering to the subject 2.0 μg/kg of the HER2 binding molecule on days 1, 8, and 15. In some embodiments, the method comprises administering to the subject 3.0 μg/kg of the HER2 binding molecule on days 1, 8, and 15. In some embodiments, the method comprises administering to the subject 4.5 μg/kg of the HER2 binding molecule on days 1, 8, and 15. In some embodiments, the method comprises administering to the subject 6.75 μg/kg of the HER2 binding molecule on days 1, 8, and 15. In some embodiments, the method comprises administering to the subject 10.0 μg/kg of the HER2 binding molecule on days 1, 8, and 15. In some embodiments, the method comprises administering to the subject 12.5 μg/kg of the HER2 binding molecule on days 1, 8, and 15. In some embodiments, the method comprises administering to the subject 15.0 μg/kg of the HER2 binding molecule on days 1, 8, and 15. In some embodiments, the method comprises administering to the subject 15.6 μg/kg of the HER2 binding molecule on days 1, 8, and 15. In some embodiments, the method comprises administering to the subject 19.5 μg/kg of the HER2 binding molecule on days 1, 8, and 15. In some embodiments, the method comprises administering to the subject 22.5 μg/kg of the HER2 binding molecule on days 1, 8, and 15. In some embodiments, the method comprises administering to the subject 33.75 μg/kg of the HER2 binding molecule on days 1, 8, and 15.

In some embodiments, the method comprises administering to the subject a composition comprising about 0.1 mg/mL to about 1 mg/mL of the HER2 binding molecule. In some embodiments, the method comprises administering to the subject a composition comprising about 0.5 mg/mL of the HER2 binding molecule. In some embodiments, the method comprises administering to the subject a composition comprising a HER2 binding molecule in a buffer comprising one or more of sodium citrate, sorbitol, and polysorbate 20. In some embodiments, the buffer has a pH in the range of about 5.3 to about 5.7. In some embodiments, the buffer has a pH of about 5.5. In some embodiments, the method comprises administering to the subject a composition comprising: (i) about 0.1 mg/mL to about 1 mg/mL of the HER2 binding molecule; (ii) about 0.5 mg/mL to about 10 mg/mL sodium citrate; (iii) about 1 mg/mL to about 100 mg/mL sorbitol; and (iv) about 0.001% (v/v) to about 0.1% (v/v) polysorbate 20; wherein the composition has a pH of about 5.3 to about 5.7. In some embodiments, the method comprises administering to the subject a composition comprising: (i) about 0.5 mg/mL of the HER2 binding molecule; (ii) about 5.2 mg/mL sodium citrate; (iii) about 36.4 mg/mL sorbitol; and (iv) about 0.02% (v/v) polysorbate 20; wherein the composition has a pH of about 5.5. In some embodiments, the method comprises administering to the subject a composition comprising: (i) about 0.5 mg/mL of the HER2 binding molecule; (ii) about 20 mM sodium citrate; (iii) about 200 mM sorbitol; and (iv) about 0.02% (v/v) polysorbate 20; wherein the composition has a pH of about 5.5.

In some embodiments, the method comprises administering to the subject a second anti-cancer agent. In some embodiments, the second anti-cancer agent is a second HER2 binding molecule. In some embodiments, the second HER2 binding molecule is trastuzumab or pertuzumab. In some embodiments, the second anti-cancer agent is trastuzumab emtansine, tucatinib, fam-trastuzumab deruxtecan, docetaxel, capecitabine, fluorouracil, or cisplatin.

In some embodiments, the cancer is a HER2-positive cancer. In some embodiments, the cancer is a HER2-positive solid cancer. In some embodiments, the cancer is an epithelial cancer. In some embodiments, the cancer is breast cancer, gastric cancer, gastroesophageal adenocarcinoma, cholangiocarcinoma, bladder cancer, gallbladder cancer, testicular cancer, ovarian cancer, uterine cancer, cervical cancer, head and neck cancer, non-small cell lung cancer, or colorectal cancer. In some embodiments, the cancer is breast cancer, gastric cancer, or gastroesophageal adenocarcinoma. In some embodiments, the cancer is cholangiocarcinoma.

In some embodiments, the cancer is relapsed or refractory to at least one other cancer therapy, or the subject is known to be intolerant of at least one other cancer therapy. In some embodiments, the cancer is relapsed or refractory to at least two prior lines of cancer therapy, or the subject is known to be intolerant of at least two prior lines of cancer therapy. In some embodiments, the cancer is relapsed or refractory to trastuzumab, pertuzumab, trastuzumab emtansine, tucatinib, fam-trastuzumab deruxtecan, docetaxel, capecitabine, fluorouracil, cisplatin, or any combination thereof.

In some embodiments, the Shiga toxin A Subunit effector polypeptide has the sequence of SEQ ID NO: 20, or a sequence that is at least 95%, at least 96%, at least 97%, at least 98%, or at least 99% identical thereto. In some embodiments, the binding region has the sequence of SEQ ID NO: 224, or a sequence that is at least at least 95%, at least 96%, at least 97%, at least 98%, or at least 99% identical thereto. In some embodiments, the Shiga toxin A subunit effector polypeptide and binding region are fused, forming a continuous polypeptide. In some embodiments, the binding molecule has the sequence of SEQ ID NO: 29, or a sequence that is at least at least 95%, at least 96%, at least 97%, at least 98%, or at least 99% identical thereto.

These and other embodiments will be described in the following detailed description, and in the attached drawings.

### BRIEF DESCRIPTION OF THE DRAWINGS

FIG. 1 is a schematic showing an illustrative and theoretical mechanism of action for the HER2 binding proteins described herein. Without being bound by any theory, it is believed that the HER2 binding proteins are capable of entering a HER2-expressing cell and inducing potent cell killing via the enzymatic and permanent inactivation of ribosomes. HER2=human epidermal growth factor receptor 2; MOA=mechanism of action; scFv=single-chain variable fragment.

FIG. 2 is a schematic showing the structure of exemplary HER2 binding molecules comprising one or more de-immunized Shiga toxin A Subunit effector polypeptides and one or more HER2 binding regions. These exemplary cell-targeting molecules each comprise a Shiga toxin effector polypeptide. A hatched rectangle depicts a furin-cleavage site disrupted by mutation(s) at the carboxy-terminus of an A1 fragment derived region of the Shiga toxin effector polypeptide. A dashed, vertical, gray line depicts a missing furin-cleavage site at the carboxy-terminus of an A1 fragment derived region of the Shiga toxin effector polypeptide. The “N” and “C” denote an amino-terminus and carboxy-terminus, respectively, of a polypeptide component of a cell-targeting molecule. In one exemplary HER2-targeting molecule, the HER2 binding region is a scFv, and the scFv is shown participating in intermolecular variable domain exchange with a neighboring scFv (bottom left).

FIG. 3 is a pictorial representation of the human HER2 protein structure with certain residues marked for their involvement in being bound by HER2 binding proteins. FIG. 3 identifies HER2 residues known to be critical for binding by certain approved anti-HER2 therapeutic monoclonal antibodies: the HER2 residues known to be critical for binding by pertuzumab binding and for trastuzumab binding are marked. The HER2 epitope bound by 115111 (SEQ ID NO:29) was mapped within the HER2 extracellular domain (ECD) to domain I; the HER2 epitope bound by pertzumab was mapped to domain II of the ECD, and the HER2 epitope bound by trastuzumab was mapped to domain IV of the ECD. FIG. 3 highlights that the HER2 epitopes bound by 115111 (SEQ ID NO:29), pertuzumab, and trastuzumab are distinct and distant from each other.

FIG. 4 is a graph showing caspase induction in cells after treatment with various doses of 115111. Caspase induction is expressed as a percentage of cells-only control in HCC1954 (a HER2-positive cell line) and MDA-MB-468 (a HER2-negative cell line).

FIG. 5 is a chart showing cytotoxic activity of 115111 on select cancer cell lines. CD₅₀=half-maximal cytotoxic concentration; iso=isotype; spec=specific; T-DM1=ado-trastuzumab emtansine. “Spec/Iso Ratio” (S/I) refers to HER2-specific monoclonal antibody-isotype control signal.

FIG. 6A-6B shows HER2 expression in various cell lines (EFM-192A, NCI-N87, SNU-216, JIMT-1, MKN-45). HER2 expression was determined using flow cytometry, relative to an isotype control antibody (i.e., to show non-specific background binding). FIG. 6A-6B also shows viability of these cell lines after treatment with various concentrations of 115111 or T-DM1. HER2=human epidermal growth factor receptor 2; T-DM1=ado-trastuzumab emtansine, *=T-DM1-resistant cell line.

FIG. 7 shows binding of 115111 to HC19954 cells, as well as 115111 toxicity in these cells. Cell viability or mean fluorescent intensity are graphed relative to concentration of 115111.

FIG. 8 is a graph showing HCC1954 cell viability as a percent of control in cells treated with vehicle plus 115111, trastuzumab plus 115111, pertuzumab plus 115111, or trastuzumab (“Traz”) plus pertuzumab (“Pertuz”) and 115111. The table below the graph shows CD₅₀ (half-maximal cytotoxic concentration) of 115111 in the presence of excess trastuzumab and pertuzumab.

FIG. 9 is a graph showing the results of an enzyme-linked immunosorbent assay (ELISA) using recombinant HER2 protein from human and cynomolgus monkey sequences and an anti-toxin monoclonal antibody was used to determine the binding affinity of 115111 to different HER2 proteins. The K_{d} was measured to be 26 ng/mL for human HER2 and 18 ng/mL for cynomolgus monkey HER2.

FIG. 10 shows pharmacokinetic (PK) data from a non-human primate study. Data was measured after a first intravenous dose using a Meso Scale Discovery-based assay. The graph shows amount of 115111 in serum at various time points post-injection.

FIG. 11 is a table that shows the dosing scheme for a 115111 GLP toxicity study in non-human primates.

FIG. 12 shows simulated human PK data using the Dedrick model. Simulations were based on the 25 μg/kg NHP PK data.

FIG. 13A-13C describe a first-in-human, open-label clinical study evaluating as monotherapy in subjects with HER2-positive locally advanced or metastatic solid cancers. FIG. 13A shows the overall study design, FIG. 13B shows Part A of the study design, and FIG. 13C shows Part B of the study design. AE=adverse event; IV=intravenous; MTD=maximum tolerated dose; RP2D=recommended phase 2 dose; OS=overall survival; PD=progressive disease; BC=breast cancer, GEA=gastroesophageal cancer; and HER2=human epidermal growth factor receptor

### 2. DETAILED DESCRIPTION

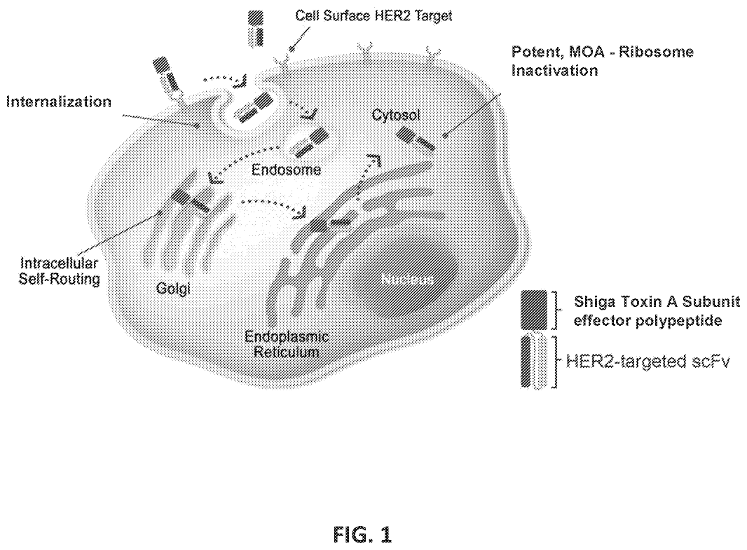

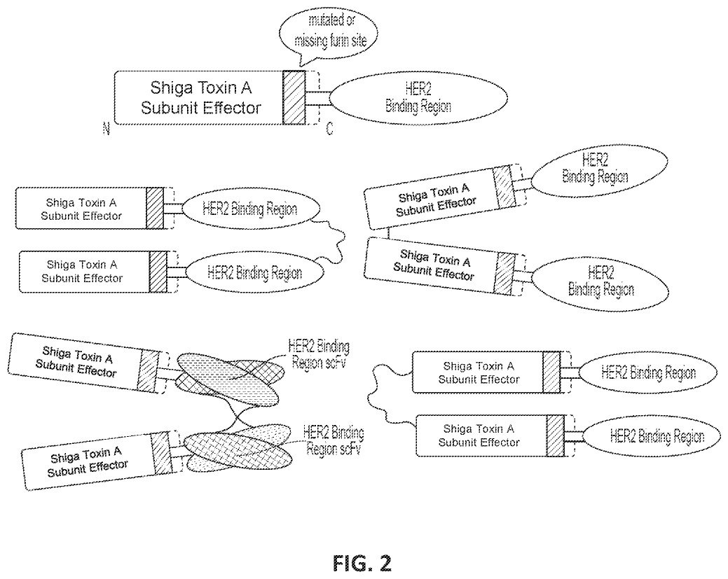

Provided herein are HER2 binding molecules, including 115111, that comprise an engineered form of a Shiga toxin A subunit genetically fused to antibody-like binding domains, and methods for use thereof. A key difference between the binding domains described herein and an antibody is that, unlike an antibody, the binding domains lack an Fc region, and therefore do not have Fc-based mechanisms of action. These molecules work through a differentiated mechanism of action involving self-routing through intracellular compartments to the cytosol, and inducing potent cell killing via the enzymatic and permanent inactivation of ribosomes (FIG. 1). This leads to programmed cell death (PCD), likely due to a ribotoxic stress response.

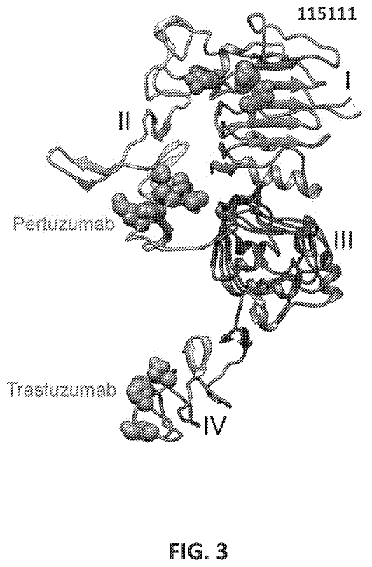

115111 (SEQ ID NO: 29) is a 55-kilodalton protein that works through a mechanism of direct cell killing via Shiga toxin effector polypeptide-mediated enzymatic ribosome inactivation, and may not be subject to resistance mechanisms that exist for tyrosine kinase inhibitors, antibody-drug conjugates, or antibody modalities. 115111 is not predicted to be a substrate of drug efflux transporters. 115111 binds an epitope on HER2 that is distinct from trastuzumab or pertuzumab, that may provide for combination potential with other HER2 targeting agents (See FIG. 3). The cytotoxic Shiga toxin A subunit effector polypeptide of 115111 is de-immunized, conferring reduced antidrug antibody development and improved tolerability in mice relative to similar molecule without the deimmunization mutations in the Shiga toxin A subunit effector polypeptide. 115111 is also described in WO 2019/0204272, which is incorporated by reference herein in its entirety, for all purposes.

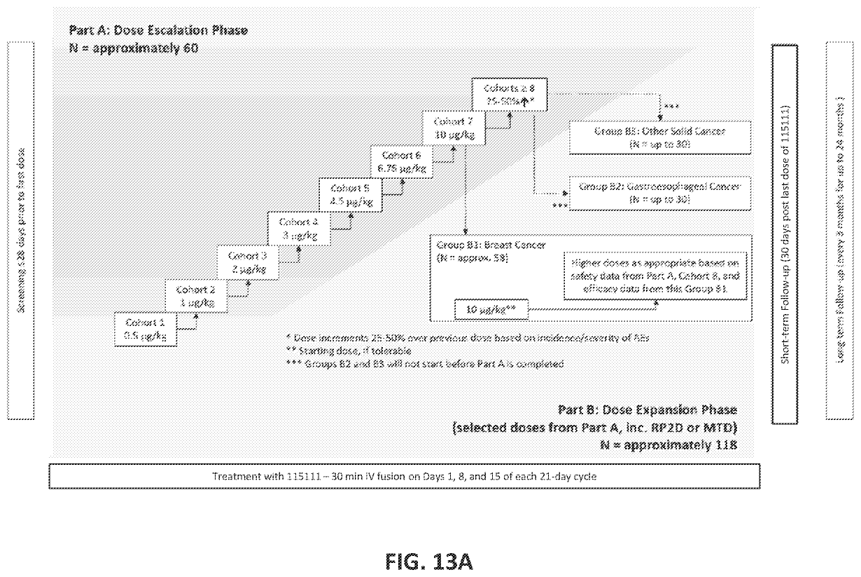

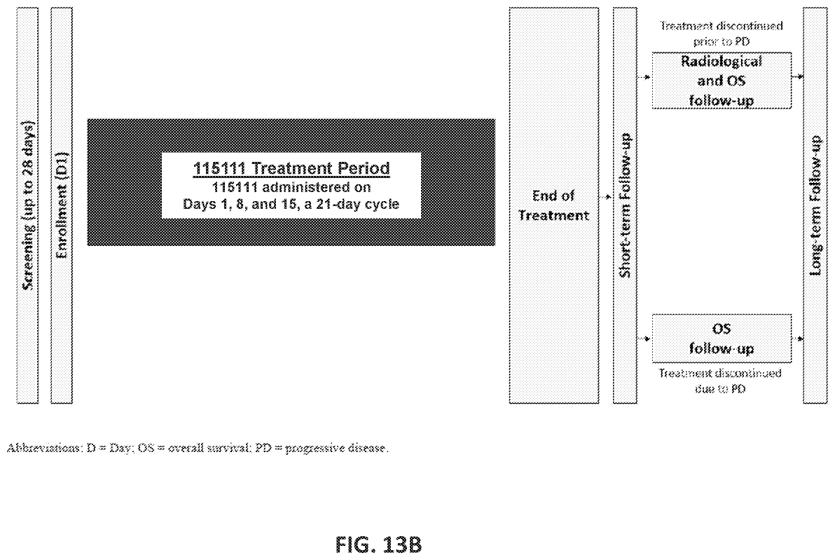

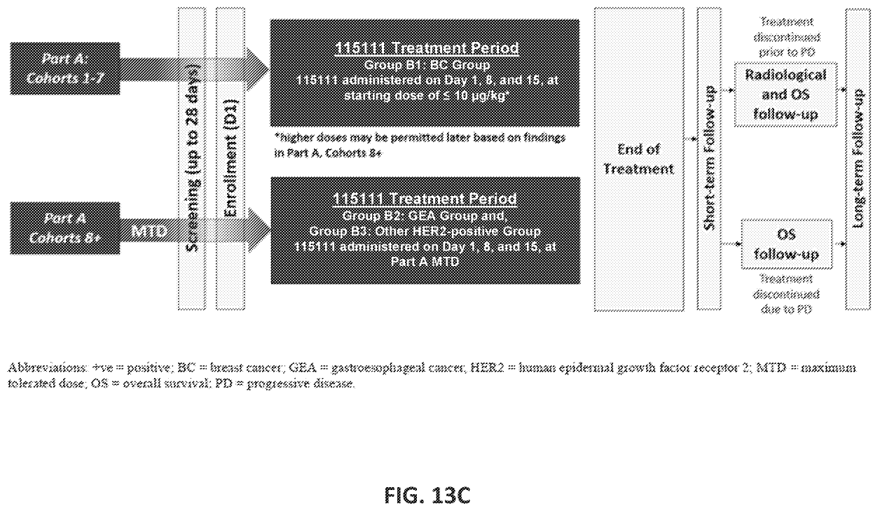

115111 is currently being evaluated as monotherapy in a first-in-human, open-label study in subjects with HER2-positive locally advanced or metastatic solid cancers (See FIGS. 13A, 13B, and 13C). The primary objective of the study is to evaluate the safety and tolerability, and to determine the maximum tolerated dose (MTD) of 115111 in subjects with advanced HER2-positive solid tumors. Secondary objectives include characterizing the pharmacokinetic (PK) profile of 115111, evaluating the tumor response to 115111, and evaluating the immunogenicity of 115111. Other objectives include correlating the pharmacodynamic (PD) markers of cancer under study with the tumor response to 115111, and, if warranted, evaluating the exposure-response relationship for using the PK, PD, safety, and tumor response variables.

Provided herein are clinical methods for the use of 115111 for treating or preventing cancer. In some embodiments, the cancer is breast cancer or gastric or gastroesophageal adenocarcinoma, such as a HER2-positive breast cancer, or a HER2-positive gastric or gastroesophageal adenocarcinoma. In some embodiments, the cancer is cholangiocarcinoma, such as HER2-positive cholangiocarcinoma.

The present invention is described more fully hereinafter using illustrative, non-limiting embodiments, and references to the accompanying figures. This invention may, however, be embodied in many different forms and should not be construed as to be limited to the embodiments set forth below. Rather, these embodiments are provided so that this disclosure is thorough and conveys the scope of the invention to those skilled in the art.

All references, articles, publications, patents, patent publications, and patent applications cited herein are incorporated by reference in their entireties for all purposes. However, disclosure of any reference, article, publication, patent, patent publication, and patent application cited herein is not, and should not be taken as, an acknowledgment or any form of suggestion that it constitutes valid prior art or form part of the common general knowledge in any country in the world.

### Definitions

In order that the present invention may be more readily understood, certain terms are defined below. Additional definitions may be found within the detailed description of the invention.

As used in the specification and the appended claims, the terms “a,” “an” and “the” include both singular and the plural referents unless the context clearly dictates otherwise.

As used in the specification and the appended claims, the term “and/or” when referring to two species, A and B, means at least one of A and B. As used in the specification and the appended claims, the term “and/or” when referring to greater than two species, such as A, B, and C, means at least one of A, B, or C, or at least one of any combination of A, B, or C (with each species in singular or multiple possibility).

As used herein, the term “a plurality of” means more than one; such as at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more.

The term “amino acid residue” or “amino acid” includes reference to an amino acid that is incorporated into a protein, polypeptide, or peptide. The term “polypeptide” includes any polymer of amino acids or amino acid residues. The term “polypeptide sequence” refers to a series of amino acids or amino acid residues which physically comprise a polypeptide. A “protein” is a macromolecule comprising one or more polypeptides or polypeptide “chains.” A “peptide” is a small polypeptide of sizes less than about a total of 15 to 20 amino acid residues. The term “amino acid sequence” refers to a series of amino acids or amino acid residues which physically comprise a peptide or polypeptide depending on the length. Unless otherwise indicated, polypeptide and protein sequences disclosed herein are written from left to right representing their order from an amino-terminus to a carboxy-terminus.

The terms “amino acid,” “amino acid residue,” “amino acid sequence,” or polypeptide sequence include naturally occurring amino acids (including L and D isostereomers) and, unless otherwise limited, also include known analogs of natural amino acids that can function in a similar manner as naturally occurring amino acids, such as selenocysteine, pyrrolysine, N′-formylmethionine, gamma-carboxyglutamate, hydroxyprolinehypusine, pyroglutamic acid, and selenomethionine. The amino acids referred to herein are described by shorthand designations as follows in Table 1.

TABLE 1
Amino Acid Nomenclature

| Name | 3-Letter | 1-Letter |
|---|---|---|
| Alanine | Ala | A |
| Arginine | Arg | R |
| Asparagine | Asn | N |
| Aspartic Acid or Aspartate | Asp | D |
| Cysteine | Cys | C |
| Glutamic Acid or Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “HER2” stands for human epidermal growth factor receptor 2. HER2 is also known as CD340, Neu, HER2/neu, receptor tyrosine-protein kinase ERBB2, or simply ERBB2. In humans, it is encoded by the ERBB2 gene. See UniProt Accession No. P04626. Amplification of the ERBB2 gene, or overexpression of HER2 has been shown to play an important role in the development and progression of certain types of breast and other cancers. HER2 is overexpressed in approximately 15-30% of breast cancers. HER2 signaling promotes cell proliferation and prevents apoptosis. Signaling pathways activated by HER2 include: mitogen-activated protein kinase (MAPK), phosphoinositide 3-kinase (PI3K/Akt), phospholipase C-gamma, protein kinase C (PKC), and signal transducer and activator of transcription (STAT) pathways. While the name HER2 might refer to multiple proteins with related structures and polypeptide sequences from various species, for the purposes of this application, the term “HER2” refers to the epidermal growth factor receptor proteins present in humans whose exact sequence might vary slightly based on the isoform and from individual to individual. For example, in some embodiments, HER2 refers to the human protein represented by the exemplary polypeptide sequences UniProt P04626 and NCBI accessions NP_004439.2, NP_001005862.1, NP_001276865.1, NP_001276866.1, and NP_001276867.1; however, different isoforms and variants exist due to splicing, polymorphisms and/or mutations.

The term “binding molecule” is used herein to describe a protein comprising at least two domains that have been joined so that they are transcribed and translated as a single unit, producing a single polypeptide. In some embodiments, a binding molecule can be a homodimeric binding molecule (comprising two identical binding molecule monomers) or a heterodimeric binding molecule (comprising two different binding molecule monomers). In some embodiments, a binding molecule is a multimeric binding molecule (comprising at least two binding molecule monomers.)

“Specific binding” or “specifically binds to” or is “specific for” a particular target or an epitope means binding that is measurably different from a non-specific interaction, e.g., binds preferentially to one target relative to another. Specific binding can be measured, for example, by determining binding of a first molecule, e.g., binding molecule, or binding domain thereof, compared to binding of a second, control molecule or binding domain thereof. In some embodiments, the control molecule that has a structure that is similar to that of the first molecule, but that does not bind to the particular target. For example, specific binding can be determined by competition with a control molecule that is similar to the target. Specific binding can include binding having an equilibrium dissociation constant (K_{D}) of at least 10⁶ M⁻¹, at least 10⁷ M⁻¹, at least 10⁸ M⁻¹, at least 10⁹ M⁻¹, or at least 10¹⁰ M⁻¹, or an affinity in the range of, for example, about 10⁶ M⁻¹ to about 10¹⁰ M⁻¹, about 10⁷ M⁻¹ to about 10¹⁰ M⁻¹, or about 10⁸ M⁻¹ to about 10¹⁰ M.

By “binding region” herein is meant a polypeptide capable of specifically binding to a target (e.g., HER2). In some embodiments, a binding region comprises a set of six complementarity-determining regions (CDRs) that, when present as part of a polypeptide sequence, specifically binds a target antigen. Thus, an “anti-HER2 binding region” binds a HER2 target as outlined herein. As is known in the art, these CDRs are generally present as a first set of variable heavy CDRs (HCDRs or VHCDRs) and a second set of variable light CDRs (LCDRs or VLCDRs), each comprising three CDRs: HCDR1, HCDR2, HCDR3 for the heavy chain and LCDR1, LCDR2 and LCDR3 for the light chain. As is understood in the art, the CDRs are separated by framework regions in each of the heavy variable and light variable regions: for the light variable region, these are (VL)FR1-LCDR1-(VL)FR2-LCDR2-(VL)FR3-LCDR3-(VL)FR4, and for the heavy variable region, these are (VH)FR1-HCDR1-(VH)FR2-HCDR2-(VH)FR3-HCDR3-(VH)FR4.

Binding regions can be embodied in multiple formats, for example, in Fab, Fv and scFv. In an “Fab” format, the set of 6 CDRs are contributed by two different polypeptide sequences, the heavy variable region (vh or VH; containing the HCDR1, HCDR2 and HCDR3) and the light variable region (vl or VL; containing the LCDR1, LCDR2 and LCDR3), with the C-terminus of the VH being attached to the N-terminus of the CH1 domain of the heavy chain and the C-terminus of the VL being attached to the N-terminus of the constant light domain (and thus forming the light chain). Heavy variable regions and light variable regions together form Fvs, which can be either scFvs or Fabs, as outlined herein. Thus, in some cases, the six CDRs of the antigen binding domain are contributed by a VH and VL. In an scFv format, the VH and VL are covalently attached, generally through the use of a linker as outlined herein, into a single polypeptide sequence, which can be either (starting from the N-terminus) VH-linker-VL or VL-linker-VH.

As used herein, the term “cytotoxic” refers to the quality of being toxic to a living cell. Cytotoxic molecules may lead to cell death, for example, by necrosis or apoptosis. The term “selective cytotoxicity” with regard to the cytotoxic activity of a molecule refers to the relative level of cytotoxicity between a target positive cell population and a non-targeted bystander cell population, which can be expressed as a ratio of the half-maximal cytotoxic concentration (CD₅₀) for a targeted cell type over the CD₅₀ for an untargeted cell type to provide a metric of cytotoxic selectivity or indication of the selectivity of killing of a targeted cell versus an untargeted cell.

As used herein, the phrases “Shiga toxin effector polypeptide,” “Shiga toxin effector polypeptide region,” and “Shiga toxin effector region” refer to a polypeptide or polypeptide region derived from at least one Shiga toxin A Subunit of a member of the Shiga toxin family wherein the polypeptide or polypeptide region is capable of exhibiting at least one Shiga toxin effector function.

As used herein, a Shiga toxin effector function is a biological activity conferred by a polypeptide region derived from a Shiga toxin A Subunit. Non-limiting examples of Shiga toxin effector functions include promoting cell entry; lipid membrane deformation; promoting cellular internalization: stimulating clathrin-mediated endocytosis; directing intracellular routing to various intracellular compartments such as, e.g., the Golgi, endoplasmic reticulum, and cytosol; directing intracellular routing with a cargo; inhibiting a ribosome function(s); catalytic activities, such as, e.g., N-glycosidase activity and catalytically inhibiting ribosomes; reducing protein synthesis, inducing caspase activity, activating effector caspases, effectuating cytostatic effects, and cytotoxicity. Shiga toxin catalytic activities include, for example, ribosome inactivation, protein synthesis inhibition, N-glycosidase activity, polynudeotide:adenosine glycosidase activity, RNAase activity, and DNAase activity. Shiga toxins are ribosome inactivating proteins (RIPs). RIPs can depurinate nucleic acids, polynudeosides, polynucleotides, rRNA, ssDNA, dsDNA, mRNA (and polyA), and viral nucleic acids. Shiga toxin catalytic activities have been observed both in vitro and in vivo. Non-limiting examples of assays for Shiga toxin effector activity measure various activities, such as, e.g., protein synthesis inhibitory activity, depurination activity, inhibition of cell growth, cytotoxicity, supercoiled DNA relaxation activity, and nuclease activity.

As used herein, the retention of Shiga toxin effector function refers to being capable of exhibiting a level of Shiga toxin functional activity, as measured by an appropriate quantitative assay with reproducibility, comparable to a wild-type, Shiga toxin effector polypeptide control (e.g. a Shiga toxin A1 fragment) or a binding molecule comprising a wild-type Shiga toxin effector polypeptide (e.g. a Shiga toxin A1 fragment) under the same conditions. For the Shiga toxin effector function of ribosome inactivation or ribosome inhibition, retained Shiga toxin effector function is exhibiting an IC₅₀ of 10,000 pM or less in an in vitro setting, such as, e.g., by using an assay known to the skilled worker and/or described herein. For the Shiga toxin effector function of cytotoxicity in a target positive cell-kill assay, retained Shiga toxin effector function is exhibiting a CD₅₀ of 1,000 nM or less, depending on the cell type and its expression of the appropriate extracellular target biomolecule, as shown, e.g., by using an assay known to the skilled worker and/or described herein.

As used herein, an “effective amount” is an amount effective for treating and/or preventing a disease, disorder, or condition as disclosed herein. In some embodiments, an effective amount is an amount or dose of a composition (e.g., a therapeutic composition, compound, or agent) that produces at least one desired therapeutic effect in a subject, such as preventing or treating a target condition or beneficially alleviating a symptom associated with the condition. In some embodiments, the effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic composition (including activity, pharmacokinetics, pharmacodynamics, and bioavailability), the physiological condition of the subject (including age, sex, disease type, disease stage, general physical condition, responsiveness to a given dosage, and type of medication), the nature of the pharmaceutically acceptable carrier or carriers in the formulation, and the route of administration. One skilled in the clinical and pharmacological arts will be able to determine an effective amount through routine experimentation, namely by monitoring a subject's response to administration of a composition and adjusting the dosage accordingly (see e.g. Remington: The Science and Practice of Pharmacy (Gennaro A, ed., Mack Publishing Co., Easton, PA, U.S., 19th ed., 1995)).

As used herein, the term “anti-cancer agent” refers to any agent useful for treating or preventing cancer. In some embodiments, the anti-cancer agent may be, for example, a protein such as an antibody (e.g., trastuzumab, pertuzumab) or other binding molecule derived therefrom, an enzyme, a signaling molecule, or a hormone. In some embodiments, the anti-cancer agent is a small molecule, such as a chemotherapeutic agent. For example, the anti-cancer agent may be an alkylating agent, or an antimetabolite. A non-limiting list of anti-cancer agents includes capecitabine, fluorouracil, anthracyclines (e.g., doxorubicin, epirubicin), taxanes (e.g., paclitaxel, docetaxel, and albumin-bound paclitaxel), platinum agents (e.g., cisplatin, carboplatin), vinorelbine, gemcitabine, ixabepilone, eribulin, or cycophosphamide.

As used herein, the term “trastuzumab” refers to a monoclonal antibody sold under the brand name Herceptin® (among others). Trastuzumab targets HER2 by binding to the juxtamembrane portion of the extracellular domain. This binding limits the receptor's ability to activate its intrinsic tyrosine kinase, which in turn, limits the activation of numerous signaling pathways that promote cell growth.

As used herein, the term “pertuzumab” refers to a monoclonal antibody sold under the brand name Perjeta® (among others). Pertuzumab binding to HER2 prevents the formation of HER2/HER3 dimers, and blocks signaling by the dimer.

HER2-positive cancers overexpress HER2 protein. HER2-positive cancers may be detected using standard immunohistochemistry (IHC) or fluorescent in situ hybridization (FISH) assays. A standard IHC test used in the art gives a score of 0 to 3+ that measures the amount of HER2 receptor protein on the surface of cells in a breast cancer tissue sample. According to this test, if the score is 0 to 1+, it's called “HER2− negative.” If the score is 2+, it's called “borderline.” A score of 3+ is called “HER2-positive.”

“Percent (%) amino acid sequence identity” with respect to a protein sequence is defined as the percentage of amino acid residues in a candidate sequence that are identical with the amino acid residues in the specific protein sequence, after aligning the sequences and introducing gaps, if necessary, to achieve the maximum percent sequence identity, and not considering any conservative substitutions as part of the sequence identity. In some embodiments, the percent sequence identity is at least about 90%, at least about 95%, at least about 96%, at least about 97%, at least about 98%, or at least about 99%.

Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full-length of the sequences being compared. One particular program is the ALIGN-2 program outlined at paragraphs [0279] to [0280] of US Pre-Grant Pub. No. 2016/0244525, hereby incorporated by reference. Another approximate alignment for nucleic acid sequences is provided by the local homology algorithm of Smith and Waterman, Advances in Applied Mathematics, 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nud. Acids Res. 14(6):6745-6763 (1986).

An example of an implementation of this algorithm to determine percent identity of a sequence is provided by the Genetics Computer Group (Madison, WI) in the “BestFit” utility application. The default parameters for this method are described in the Wisconsin Sequence Analysis Package Program Manual, Version 8 (1995) (available from Genetics Computer Group, Madison, WI). Another method of establishing percent identity is to use the MPSRCH package of programs copyrighted by the University of Edinburgh, developed by John F. Collins and Shane S. Sturrok, and distributed by IntelliGenetics, Inc. (Mountain View, CA). From this suite of packages, the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the internet address located by placing http:// in front of blast.ncbi.nlm.nih.gov/Blast.cgi.

The degree of identity between a first protein sequence and the candidate amino acid sequence is calculated as the number of exact matches in an alignment of the two sequences, divided by the length of the first amino acid sequence, or the length of the parental sequence, whichever is the shortest. The result is expressed in percent identity.

As used herein, the term “continuous polypeptide” refers to a single polypeptide comprising a binding region and a Shiga toxin A effector that are fused by a peptide bond.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (VH-linker-VL or VL-linker-VH). In general, the linker is a scFv linker as is generally known in the art and discussed above.

The term “VHH” is used herein to describe a single domain antibody, an autonomous heavy domain antibody variable domain, or a binding region having structural and/or sequence similarity to, for example, a variable antigen-binding domain heavy-chain antibody from a camelid (camel, dromedary, llama, alpaca, etc.) or to an immunoglobulin new antigen receptor (IgNAR) of a cartilaginous fish (e.g., a shark). In some embodiments, a VHH may be very small in size, for example about 12 to about 15 kDa. A VHH may also be referred to herein as a “nanobody.”

By “linker” herein is meant a domain linker that joins two protein domains together, such as are used in scFv and/or other protein and protein fusion structures. For example, a “binding region linker” may be used to link a Shiga Toxin A subunit effector polypeptide with a binding region, and a “scFv linker” may be used to link the VH and the VL in an scFv. Generally, there are a number of suitable linkers that can be used, including traditional peptide bonds, generated by recombinant techniques that allows for recombinant attachment of the two domains with sufficient length and flexibility to allow each domain to retain its biological function. In some embodiments, the linker peptide can predominantly include the following amino acid residues: Gly, Ser, Ala, or Thr. The linker peptide should have a length that is adequate to link two molecules in such a way that they assume the correct conformation relative to one another so that they retain the desired activity. In some embodiments, the linker is from about 1 to about 50 amino acids in length. In some embodiments, the linker is from about 1 to about 30 amino acids in length. In one embodiment, linkers of 1 to 20 amino acids in length can be used, with from about 5 to about 10 amino acids finding use in some embodiments. Useful linkers include glycine-serine polymers, including for example (GS)ₙ (SEQ ID NO: 187), (GSGGS)ₙ (SEQ ID NO: 188), (GGGGS)ₙ (SEQ ID NO: 189), and (GGGS)ₙ (SEQ ID NO: 190), where n is an integer of at least one (and generally from 3 to 4), glycine-alanine polymers, alanine-serine polymers, and other flexible linkers. Alternatively, a variety of non-proteinaceous polymers, including but not limited to polyethylene glycol (PEG), polypropylene glycol, polyoxyalkylenes, or copolymers of polyethylene glycol and polypropylene glycol, can find use as linkers. Other linker sequences can include any sequence of any length of CLCH1 domain but not all residues of CL/CH1 domain; for example, the first 5-12 amino acid residues of the CL/CH1 domains. Linkers can also be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences can also be derived from other proteins such as Ig-like proteins (e.g., TCR, FcR, KIR), hinge region-derived sequences, and other natural sequences from other proteins. While any suitable linker can be used, some embodiments utilize a glycine-serine polymer, including for example (GS)ₙ (SEQ ID NO: 187), (GSGGS)ₙ (SEQ ID NO: 188), (GGGGS)ₙ (SEQ ID NO: 189), and (GGGS)ₙ (SEQ ID NO: 190), where n is an integer of at least one (and generally from 2 to 3 to 4 to 5). “scFv linkers” generally include these glycine-serine polymers.

The term “antibody” is used in the broadest sense and includes, for example, an intact immunoglobulin or an antigen binding portion of an immunoglobulin or an antigen binding protein related or derived from an immunoglobulin. Intact antibody structural units often comprise a tetrameric protein. Each tetramer is typically composed of two identical pairs of polypeptide chains, each pair having one “light” chain (typically having a molecular weight of about 25 kDa) and one “heavy” chain (typically having a molecular weight of about 50- to 70 kDa). Human immunoglobulin light chains can be classified as having kappa or lambda light chains. In some embodiments, provided herein are antibody structures comprising antigen binding domains (e.g. antibody heavy and/or light chains) that generally are based on the IgG class, which has several subclasses, including, but not limited to IgG1, IgG2, IgG3, and IgG4. In general, IgG1 has different allotypes with polymorphisms at 356 (D or E), IgG2 and 358 (L or M). The sequences depicted herein use the 356D/358M allotype, however the other allotype is included herein. That is, any sequence inclusive of an IgG1 Fc domain included herein can have 356E/358L replacing the 356D/358M allotype. IgG4 are used more frequently than IgG3.

By “Fc” or “Fc region” or “Fc domain” as used herein is meant the polypeptide comprising the constant region of an antibody excluding the first constant region immunoglobulin domain (e.g., CH1) and in some cases, part of the hinge. For IgG, the Fc domain comprises immunoglobulin domains CH2 and CH3 (Cγ2 and Cγ3) and the lower hinge region between CH1 (Cγ1) and CH2 (Cγ2). Although the boundaries of the Fc region can vary, the human IgG heavy chain Fc region is usually defined to include residues C226 or P230 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Thus, the “Fc domain” includes the —CH2-CH3 domain, and optionally a hinge domain (hinge-CH2-CH3). In some embodiments, as is more fully described below, amino acid modifications are made to the Fc region, for example to alter binding to one or more FcγR receptors or to the FcRn receptor.

By “variable domain” as used herein is meant the region of an immunoglobulin that comprises one or more Ig domains substantially encoded by any of the Vκ (V.kappa), Vλ (V.lambda), and/or VH genes that make up the kappa, lambda, and heavy chain immunoglobulin genetic loci respectively. Thus a “variable heavy domain” or a “heavy chain variable domain” comprises (VH)FR1-HCDR1-(VH)FR2-HCDR2-(VH)FR3-HCDR3-(VH)FR4 and a “variable light domain” or a “light chain variable domain” comprises (VL)FR1-LCDR1-(VL)FR2-LCDR2-(VL)FR3-LCDR3-(VL)FR4.

Each VH and VL is composed of three hypervariable regions (“complementary determining regions,” “CDRs”) and four FRs, arranged from amino-terminus to carboxy-terminus in the following order. FR1-CDR1-FR2-CDR2-FR3-CDR3-FR4. The CDRs contribute to the formation of the antigen-binding, or more specifically, epitope binding site of antibodies.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs are described below.

As will be appreciated by those in the art, the exact numbering and placement of the CDRs can be different among different numbering systems. A useful comparison of CDR numbering is as below (Table 2), see Lafranc et al., Dev. Comp. Immunol. 27(1):55-77 (2003):

TABLE 2
Antibody CDR Nomenclature

| | Kabat + | | | | | |
|---|---|---|---|---|---|---|
| | Chothia | IMGT | Kabat | AbM | Chothia | Contact |
| HCDR1 | 26-35 | 27-38 | 31-35 | 26-35 | 26-32 | 30-35 |
| HCDR2 | 50-65 | 56-65 | 50-65 | 50-58 | 52-56 | 47-58 |
| HCDR3 | 95-102 | 105-117 | 95-102 | 95-102 | 95-102 | 93-101 |
| LCDR1 | 24-34 | 27-38 | 24-34 | 24-34 | 24-34 | 30-36 |
| LCDR2 | 50-56 | 56-65 | 50-56 | 50-56 | 50-56 | 46-55 |
| LCDR3 | 89-97 | 105-117 | 89-97 | 89-97 | 89-97 | 89-96 |

As used herein, the phrase “derived from” when referring to a polypeptide or polypeptide region means that the polypeptide or polypeptide region comprises amino acid sequences originally found in a “parental” protein and which may now comprise some amino acid residue additions, deletions, truncations, rearrangements, or other alterations relative to the original polypeptide or polypeptide region as long as a some function(s) and a structure(s) of the “parental” molecule are substantially conserved. The skilled worker will be able to identify a parental molecule from which a polypeptide or polypeptide region was derived using techniques known in the art, e.g., protein sequence alignment software.

### HER2 Binding Regions

In some embodiments, a binding region of a cell-targeting molecule is a cell-targeting component, such as, e.g., a domain, molecular moiety, or agent, capable of binding specifically to an extracellular part of a HER2 molecule on a cell surface with high affinity. Numerous types of binding regions known to skilled worker or which may be discovered by the skilled worker using techniques known in the art may be used in the binding molecules described herein. For example, any cell-targeting component that exhibits the requisite binding characteristics described herein may be used as the binding region in some embodiments of the cell-targeting molecules described herein.

An “extracellular part” of a target biomolecule (e.g., HER2) refers to a portion of its structure exposed to the extracellular environment when the molecule is physically coupled to a cell, such as, e.g., when the target biomolecule is expressed at a cellular surface by the cell. In this context, exposed to the extracellular environment means that part of the target biomolecule is accessible by, e.g., an antibody or at least a binding moiety smaller than an antibody such as a single-domain antibody domain, a Nanobody®, a heavy-chain antibody domain derived from camelids or cartilaginous fishes, a single-chain variable fragment, or any number of engineered alternative scaffolds to immunoglobulins. The exposure to the extracellular environment of or accessibility to a part of target biomolecule physically coupled to a cell may be empirically determined by the skilled worker using methods well known in the art.

A binding region of a HER2 binding molecule may be, e.g., a ligand, peptide, immunoglobulin-type binding region, monoclonal antibody, engineered antibody derivative, or engineered alternative to antibodies. In some embodiments, the binding region may comprise an immunoglobulin-type binding region. The term “immunoglobulin-type binding region” as used herein refers to a polypeptide region capable of binding one or more target biomolecules, such as an antigen or epitope. Binding regions may be functionally defined by their ability to bind to target molecules. Immunoglobulin-type binding regions are commonly derived from antibody or antibody-like structures; however, alternative scaffolds from other sources are contemplated within the scope of the term. In some embodiments, the binding region may comprise an immunoglobulin binding region derived from antibody or antibody-like structure.

Immunoglobulin (Ig) proteins have a structural domain known as an Ig domain. Ig domains range in length from about 70-110 amino acid residues and possess a characteristic Ig-fold, in which typically 7 to 9 antiparallel beta strands arrange into two beta sheets which form a sandwich-like structure. The Ig fold is stabilized by hydrophobic amino acid interactions on inner surfaces of the sandwich and highly conserved disulfide bonds between cysteine residues in the strands. Ig domains may be variable (IgV or V-set), constant (IgC or C-set) or intermediate (IgI or I-set). Some Ig domains may be associated with a complementarity determining region (CDR), also called a “complementary determining region,” which is important for the specificity of antibodies binding to their epitopes. Ig-like domains are also found in non-immunoglobulin proteins and are classified on that basis as members of the Ig superfamily of proteins. The HUGO Gene Nomenclature Committee (HGNC) provides a list of members of the Ig-like domain containing family.

An immunoglobulin-type binding region may be a polypeptide sequence of an antibody or antigen-binding fragment thereof wherein the amino acid sequence has been varied from that of a native antibody or an Ig-like domain of a non-immunoglobulin protein, for example by molecular engineering or selection by library screening. Because of the relevance of recombinant DNA techniques and in vitro library screening in the generation of immunoglobulin-type binding regions, antibodies can be redesigned to obtain desired characteristics, such as smaller size, cell entry, or other improvements for in vivo and/or therapeutic applications. The possible variations are many and may range from the changing of just one amino acid to the complete redesign of, for example, a variable region. Typically, changes in the variable region will be made in order to improve the antigen-binding characteristics, improve variable region stability, or reduce the potential for immunogenic responses.

There are numerous immunoglobulin-type binding regions contemplated as components of the binding molecules described herein. In some embodiments, the immunoglobulin-type binding region is derived from an immunoglobulin binding region, such as an antibody paratope capable of binding an extracellular target biomolecule. In some embodiments, the immunoglobulin-type binding region comprises an engineered polypeptide not derived from any immunoglobulin domain but which functions like an immunoglobulin binding region by providing high-affinity binding to an extracellular target biomolecule. This engineered polypeptide may optionally include polypeptide scaffolds comprising, consisting of, or consisting essentially of complementary determining regions from immunoglobulins as described herein.

Numerous other binding regions may be useful for targeting polypeptides to specific cell-types via their high-affinity binding characteristics. In some embodiments, the binding region of a cell-targeting molecule is selected from the group which includes autonomous VH domains, single-domain antibody domains (sdAbs), heavy-chain antibody domains derived from camelids (VHH fragments or VH domain fragments), heavy-chain antibody domains derived from camelid VHH fragments or VH domain fragments, heavy-chain antibody domains derived from cartilaginous fishes, immunoglobulin new antigen receptors (IgNARs), VNAR fragments, single-chain variable (scFv) fragments, Nanobodies®, Fd fragments consisting of the heavy chain and (H1 domains, single chain FV-CH3 minibodies, dimeric CH2 domain fragments (CH2D), FC antigen binding domains (Fcabs), isolated complementary determining region 3 (CDR3) fragments, constrained framework region 3, CDR3, framework region 4 (FR3-CDR3-FR4) polypeptides, small modular immunopharmaceutical (SMIP) domains, scFv-Fc fusions, multimerizing scFv fragments (diabodies, triabodies, tetrabodies), disulfide stabilized antibody variable (Fv) fragments, disulfide stabilized antigen-binding (Fab) fragments consisting of the VL, VH, CL and C1 domains, bivalent Nanobodies®, bivalent minibodies, bivalent F(ab′)2 fragments (Fab dimers), bispecific tandem VHH fragments, bispecific tandem scFv fragments, bispecific Nanobodies®, bispecific minibodies, and any genetically manipulated counterparts of the foregoing that retain its paratope and binding function. For example, a cell-targeting molecule may comprise a binding region that comprises, consists essentially of, or consists of one or more of: an antibody variable fragment, a single-domain antibody fragment, a single-chain variable fragment, a Fd fragment, an antigen-binding fragment, an autonomous VH domain, a VHH fragment derived from a camelid antibody, a heavy-chain antibody domain derived from a cartilaginous fish antibody, a VNAR fragment, and an immunoglobulin new antigen receptor. In some embodiments, the binding region comprises, consists essentially of, or consists of a single-chain variable fragment and/or a VHH fragment derived from a camelid antibody. In some embodiments, the binding region comprises, consists essentially of, or consists of a single-chain variable fragment. In some embodiments, the binding region comprises, consists essentially of, or consists of a VHH fragment derived from a camelid antibody.

In some embodiments, the binding region comprises an engineered, alternative scaffold to immunoglobulin domains. Engineered alternative scaffolds are known in the art which exhibit similar functional characteristics to immunoglobulin-derived structures, such as high-affinity and specific binding of target biomolecules, and may provide improved characteristics to certain immunoglobulin domains, such as, e.g., greater stability or reduced immunogenicity. Generally, alternative scaffolds to immunoglobulins are less than 20 kilodaltons, consist of a single polypeptide chain, lack cysteine residues, and exhibit relatively high thermodynamic stability.

In some embodiments, the binding region comprises an alternative scaffold selected from the group which includes autonomous VH domains, single-domain antibody domains (sdAbs), heavy-chain antibody domains derived from camelids (VHH fragments or VH domain fragments), heavy-chain antibody domains derived from camelid VHH fragments or VH domain fragments, heavy-chain antibody domains derived from cartilaginous fishes, immunoglobulin new antigen receptors (IgNARs), VNAR fragments, single-chain variable (scFv) fragments, Nanobodies®, Fd fragments consisting of the heavy chain and C1 domains, permutated Fvs (pFv), single chain FV-CH3 minibodies, dimeric CH2 domain fragments (CH2D), FC antigen binding domains (Fcabs), isolated complementary determining region 3 (CDR3) fragments, constrained framework region 3, CDR3, framework region 4 (FR3-CDR3-FR4) polypeptides, small modular immunopharmaceutical (SMIP) domains, scFv-Fc fusions, multimerizing scFv fragments (diabodies, triabodies, tetrabodies), disulfide stabilized antibody variable (Fv) fragments, disulfide stabilized antigen binding (Fab) fragments consisting of the VL, VH, CL and Cn1 domains, bivalent Nanobodies®, bivalent minibodies, bivalent F(ab′) 2 fragments (Fab dimers), bispecific tandem VHH fragments, bispecific tandem scFv fragments, bispecific Nanobodies®, bispecific minibodies, and any genetically manipulated counterparts of the foregoing that retains its binding functionality.

In addition to alternative antibody formats, antibody-like binding abilities may be conferred by non-proteinaceous compounds, such as, e.g., oligomers, RNA molecules, DNA molecules, carbohydrates, and glycocalyxcalixarenes or partially proteinaceous compounds, such as, e.g., phenol-formaldehyde cyclic oligomers coupled with peptides and calixarene-peptide compositions.

In some embodiments, the binding region is an immunoglobulin-type HER2 binding region such as a HER2-binding monoclonal antibody or derivative thereof. For example, the binding region may be derived from one or more of the following antibodies: anti-ErbB2, 4D5, 2C4, 7F3, 7C2, mumAb 4D5, chmAb 4D5, (rhu)mAb 4D5, huMAb4D5-I, huMAb4D5-2, huMAb4D5-3, huMAb4D5-4, huMAb4D5-5, huMAb4D5-6, huMAb4D5-7, huMAb4D5-8, trastuzumab, humanized 520C9, 4D5Fc8, hingeless rhu4D5, non-glycosylated rhu4D5 with mutated cysteine residues, pertuzumab, and humanized 2C4.

In some embodiments, the cell-targeting molecule comprises a binding region comprising an immunoglobulin-type polypeptide (e.g., an immunoglobulin polypeptide) selected for specific and high-affinity binding to human HER2 and/or the cellular surface of a HER2-positive cell. In some embodiments, the binding region comprises at least one heavy chain variable (VH) domain; and/or at least one light chain variable (VL) domain. As described herein, the at least one heavy-chain variable domain polypeptide may be linked to the at least one light-chain variable domain polypeptide by a linker (such as a linker or inter-domain linker described herein). In some embodiments, the binding region comprises a single-domain antibody fragment, such as, e.g., only a heavy chain variable (VHH) domain (e.g., as derived from a camelid antibody).

The binding region of the cell-targeting molecule may be defined by reference to its CDRs, such as those defined in SEQ ID NOs: 45-74. These sequences are provided below in Table 3.

TABLE 3
CDR Sequences

| Description | Sequence | SEQ ID NO |
|---|---|---|
| vhCDR1 | DTYIH | 45 |
| vhCDR2 | RIYPTNGYTRYADSVKG | 46 |
| vhCDR3 | WGGDGFYAMDY | 47 |
| VlCDR1 | RASQDVNTAVA | 48 |
| VlCDR2 | SASFLYS | 49 |
| VlCDR3 | QQHYTTPPT | 50 |
| vhCDR1 | SYWIA | 51 |
| vhCDR2 | LIYPGDSDTKYSPSFQG | 52 |
| vhCDR3 | HDVGYCSSSNCAKWPEYFQH | 53 |
| VlCDR1 | SGSSSNIGNNYVS | 54 |
| VlCDR2 | SASYRYT | 55 |
| VlCDR3 | QQYYIYPYT | 56 |
| vhCDR1 | NYGMN | 57 |
| vhCDR2 | WINTSTGESTFADDFKG | 58 |
| vhCDR3 | WEVYHGYVPY | 59 |
| VlCDR1 | KASQDVYNAVA | 60 |
| VlCDR2 | SASSRYT | 61 |
| VlCDR3 | QQHFRTPFT | 62 |
| vhCDR1 | DYTMD | 63 |
| vhCDR2 | DVNPNSGGSIYNQRFKG | 64 |
| vhCDR3 | NLGPSFYFDY | 65 |
| VlCDR1 | KASQDVSIGVA | 66 |
| VlCDR2 | SASYRYT | 67 |
| VlCDR3 | QQYYIYPYT | 68 |
| vhCDR1 | INTMG | 69 |
| vhCDR2 | LISSIGDTYYADSVKG | 70 |
| vhCDR3 | FRTAAQGTDY | 71 |
| VlCDR1 | SCGMG | 72 |
| VlCDR2 | RISGDGDTWHKESVKG | 73 |
| VlCDR3 | CYNLETY | 74 |

In some embodiments, the binding region comprises a polypeptide(s) selected from the group consisting of: a) a heavy chain variable (VH) domain comprising (i) a HCDR1 comprising or consisting essentially of one of the amino acid sequences as shown in SEQ ID NO:45, SEQ ID NO:51, SEQ ID NO:57 or SEQ ID NO:63; (ii) a HCDR2 comprising or consisting essentially of one of the amino acid sequence as shown in SEQ ID NO:46, SEQ ID NO:52, SEQ ID NO:58, or SEQ ID NO:64; and (iii) a HCDR3 comprising or consisting essentially of one of the amino acid sequence as shown in SEQ ID NO:47, SEQ ID NO:53, SEQ ID NO:59, or SEQ ID NO:65; and/or b) a light chain variable (VL) domain comprising (i) a LCDR1 comprising or consisting essentially of one of the amino acid sequence as shown in SEQ ID NO:48, SEQ ID NO:54, SEQ ID NO:60, or SEQ ID NO:66; (ii) a LCDR2 comprising or consisting essentially of one of the amino acid sequence as shown in SEQ ID NO:49, SEQ ID NO:55, SEQ ID NO:61 or SEQ ID NO:67; and (iii) a LCDR3 comprising or consisting essentially of one of the amino acid sequence as shown in SEQ ID NO:50, SEQ ID NO:56, SEQ ID NO:62, or SEQ ID NO:68. In some embodiments, the binding region comprises at least one heavy-chain variable domain polypeptide comprising (i) the HCDR1, HCDR2, and HCDR3 amino acid sequences shown in SEQ ID NOs: 51, SEQ ID NO:52, and SEQ ID NO:53, respectively; (ii) the HCDR1, HCDR2, and HCDR3 amino acid sequences shown in SEQ ID NO:57, SEQ ID NO:58, and SEQ ID NO:59, respectively; or (iii) the HCDR1, HCDR2, and HCDR3 amino acid sequences shown in SEQ ID NO:63, SEQ ID NO:64, and SEQ ID NO:65, respectively. In some embodiments, the binding region comprises at least one light-chain variable domain polypeptide comprising (i) the LCDR1, LCDR2, and LCDR3 amino acid sequences shown in SEQ ID NO:54, SEQ ID NO:55, and SEQ ID NO:56, respectively; (ii) the LCDR1, LCDR2, and LCDR3 amino acid sequences shown in SEQ ID NO:60, SEQ ID NO:61, and SEQ ID NO:62, respectively; or (iii) the LCDR1, LCDR2, and LCDR3 amino acid sequences shown in SEQ ID NO:66, SEQ ID NO:67, and SEQ ID NO:68, respectively.

In some embodiments, the binding region comprises at least one heavy-chain variable domain polypeptide comprising (i) the HCDR1, HCDR2, and HCDR3 amino acid sequences shown in SEQ ID NOs: 51, SEQ ID NO:52, and SEQ ID NO:53, respectively; (ii) the HCDR1, HCDR2, and HCDR3 amino acid sequences shown in SEQ ID NO:57, SEQ ID NO:58, and SEQ ID NO:59, respectively; or (iii) the HCDR1, HCDR2, and HCDR3 amino acid sequences shown in SEQ ID NO:63, SEQ ID NO:64, and SEQ ID NO:65, respectively; and at least one light-chain variable domain polypeptide comprising (i) the LCDR1, LCDR2, and LCDR3 amino acid sequences shown in SEQ ID NO:54, SEQ ID NO:55, and SEQ ID NO:56, respectively; (ii) the LCDR1, LCDR2, and LCDR3 amino acid sequences shown in SEQ ID NO:60, SEQ ID NO:61, and SEQ ID NO:62, respectively; or (iii) the LCDR1, LCDR2, and LCDR3 amino acid sequences shown in SEQ ID NO:66, SEQ ID NO:67, and SEQ ID NO:68, respectively. For example, the binding region may comprises at least one heavy-chain variable domain polypeptide comprising (i) the HCDR1, HCDR2, and HCDR3 amino acid sequences shown in SEQ ID NOs: 51, SEQ ID NO:52, and SEQ ID NO:53, respectively; and at least one light-chain variable domain polypeptide comprising: (i) the LCDR1, LCDR2, and LCDR3 amino acid sequences shown in SEQ ID NO:54, SEQ ID NO:55, and SEQ ID NO:56, respectively. For example, the binding region may comprises at least one heavy-chain variable domain polypeptide comprising (i) the HCDR1, HCDR2, and HCDR3 amino acid sequences shown in SEQ ID NOs: 57, SEQ ID NO:58, and SEQ ID NO:59, respectively; and at least one light-chain variable domain polypeptide comprising (i) the LCDR1, LCDR2, and LCDR3 amino acid sequences shown in SEQ ID NO:60, SEQ ID NO:61, and SEQ ID NO:62, respectively. For example, the binding region may comprises at least one heavy-chain variable domain polypeptide comprising (i) the HCDR1, HCDR2, and HCDR3 amino acid sequences shown in SEQ ID NOs: 63, SEQ ID NO:64, and SEQ ID NO:65, respectively; and at least one light-chain variable domain polypeptide comprising (i) the LCDR1, LCDR2, and LCDR3 amino acid sequences shown in SEQ ID NO:66, SEQ ID NO:67, and SEQ ID NO:68, respectively. The binding region having these CDRs may be an immunoglobulin binding region comprising a single-chain variable fragment.

In some embodiments, the binding region comprises a polypeptide(s) selected from the group consisting of: a) a heavy chain only variable (VHH) domain comprising (i) a HCDR1 comprising or consisting essentially of the amino acid sequences as shown in SEQ ID NO:69 or SEQ ID NO:72; (ii) a HCDR2 comprising or consisting essentially of the amino acid sequence as shown in SEQ ID NO:70 or SEQ ID NO:73; and/or (iii) a HCDR3 comprising or consisting essentially of the amino acid sequence as shown in SEQ ID NO:71 or SEQ ID NO:74. In some embodiments, the binding region comprises a polypeptide(s) selected from the group consisting of: a) a heavy chain only variable (VHH) domain comprising (i) a HCDR1 comprising or consisting essentially of the amino acid sequences as shown in SEQ ID NO:69 or SEQ ID NO:72; (ii) a HCDR2 comprising or consisting essentially of the amino acid sequence as shown in SEQ ID NO:70 or SEQ ID NO:73; and (iii) a HCDR3 comprising or consisting essentially of the amino acid sequence as shown in SEQ ID NO:71 or SEQ ID NO:74. The binding region having these CDRs may be an immunoglobulin binding region comprising a heavy chain only variable (VHH) domain derived from a camelid antibody.

In some embodiments, the binding region comprises, consists essentially of, or consists of an amino acid sequence that is at least 85% (such as at least 90%, 91%, 92%, 93%, 94%, 95%, 96%, 97%, 98%, 99% or more) identical to the amino acid sequence of: amino acids 269 to 501 of SEQ ID NO:24; amino acids 269 to 513 of SEQ ID NO:25; amino acids 269 to 499 of SEQ ID NO: 26 or SEQ ID NO:27; amino acids; amino acids 269-520 of SEQ ID NO:28; amino acids 269 to 519 of SEQ ID NO:29 or SEQ ID NO:30; amino acids 268 to 386 of SEQ ID NO: 31; amino acids 269 to 499 of SEQ ID NO:32; amino acids 269 to 499 of SEQ ID NO:33; amino acids 253 to 370 of SEQ ID NO:34; amino acids 253 to 367 of SEQ ID NO:35; amino acids 269 to 514 of SEQ ID NO:36; amino acids 268 to 498 of SEQ ID NO:99; amino acids 268 to 499 of SEQ ID NO: 100; amino acids 268 to 500 of SEQ ID NO:97; amino acids 268 to 512 of SEQ ID NO:98; amino acids 268 to 518 of SEQ ID NO: 102 or SEQ ID NO: 103; amino acids 268-519 of SEQ ID NO: 101; amino acids 267 to 384 of SEQ ID NO: 104; amino acids 268 to 498 of SEQ ID NO: 105; amino acids 252 to 370 of SEQ ID NO: 106; amino acids 252 to 366 of SEQ ID NO: 107; and amino acids 268 to 513 of SEQ ID NO: 108. In some embodiments, the binding region comprises, consists essentially of, or consists of an amino acid sequence that is at least 85% (such as at least 90%, 91%, 92%, 93%, 94%, 95%, 96%, 97%, 98%, 99% or more) identical to the amino acid sequence of: amino acids 269 to 513 of SEQ ID NO:25; amino acids 269 to 499 of SEQ ID NO:26; amino acids 269 to 519 of SEQ ID NO:29 or SEQ ID NO:30; amino acids 268 to 386 of SEQ ID NO:31; amino acids 253 to 370 of SEQ ID NO:34; amino acids 253 to 367 of SEQ ID NO:35; or amino acids 269 to 514 of SEQ ID NO:36. In some embodiments, the binding region comprises, consists essentially of, or consists of an amino acid sequence that is at least 85% (such as at least 90%, 91%, 92%, 93%, 94%, 95%, 96%, 97%, 98%, 99% or more) identical to amino acids 269 to 519 of SEQ ID NO:29 or SEQ ID NO:30.

In some embodiments, the binding region comprises, consists essentially of, or consists of the polypeptide represented by any one of the following polypeptide sequences: amino acids 269 to 501 of SEQ ID NO:24; amino acids 269 to 513 of SEQ ID NO:25; amino acids 269 to 499 of SEQ ID NO: 26 or SEQ ID NO:27; amino acids; amino acids 269-520 of SEQ ID NO:28; amino acids 269 to 519 of SEQ ID NO:29 or SEQ ID NO:30; amino acids 268 to 386 of SEQ ID NO:31; amino acids 269 to 499 of SEQ ID NO:32; amino acids 269 to 499 of SEQ ID NO:33; amino acids 253 to 370 of SEQ ID NO:34; amino acids 253 to 367 of SEQ ID NO:35; amino acids 269 to 514 of SEQ ID NO:36 amino acids 268 to 498 of SEQ ID NO:99; amino acids 268 to 499 of SEQ ID NO: 100; amino acids 268 to 500 of SEQ ID NO:97; amino acids 268 to 512 of SEQ ID NO:98; amino acids 268 to 518 of SEQ ID NO: 102 or SEQ ID NO: 103; amino acids 268-519 of SEQ ID NO: 101; amino acids 267 to 384 of SEQ ID NO: 104; amino acids 268 to 498 of SEQ ID NO: 105; amino acids 252 to 370 of SEQ ID NO: 106; amino acids 252 to 366 of SEQ ID NO: 107; and amino acids 268 to 513 of SEQ ID NO: 108. In some embodiments, the binding region comprises, consists essentially of, or consists of the polypeptide represented by any one of the following polypeptide sequences: amino acids 269 to 513 of SEQ ID NO:25; amino acids 269 to 499 of SEQ ID NO:26; amino acids 269 to 519 of SEQ ID NO:29 or SEQ ID NO:30; amino acids 268 to 386 of SEQ ID NO:31; amino acids 253 to 370 of SEQ ID NO:34; amino acids 253 to 367 of SEQ ID NO:35; and amino acids 269 to 514 of SEQ ID NO:36. In some embodiments, the binding region comprises, consists essentially of, or consists of the polypeptide represented by amino acids 269 to 519 of SEQ ID NO:29 or SEQ ID NO:30. In some embodiments, the binding region comprises, consists essentially of, or consists of the polypeptide represented by amino acids 269 to 519 of SEQ ID NO:29, amino acids 268 to 386 of SEQ ID NO:31; amino acids 253 to 370 of SEQ ID NO:34; or amino acids 253 to 367 of SEQ ID NO:35. In some embodiments, the binding region comprises, consists essentially of, or consists of the polypeptide represented by amino acids 269 to 519 of SEQ ID NO:29. In certain, embodiments, the binding region comprises, consists essentially of, or consists of the polypeptide represented by amino acids 268 to 386 of SEQ ID NO:31. In some embodiments, the binding region comprises, consists essentially of, or consists of the polypeptide represented by amino acids 253 to 370 of SEQ ID NO: 34. In some embodiments, the binding region comprises, consists essentially of, or consists of the polypeptide represented by amino acids 253 to 367 of SEQ ID NO: 35. In some embodiments, the binding region comprises, consists essentially of, or consists of the polypeptide represented by amino acids 269 to 514 of SEQ ID NO: 36.

In some embodiments, the binding region comprises at least one heavy chain variable (VH) domain comprising, consisting essentially of, or consisting of an amino acid sequence that is at least 85% (such as at least 90%, 91%, 92%, 93%, 94%, 95%, 96%, 97%, 98%, 99% or more) identical to the amino acid sequence shown in any one of: amino acids 253 to 367 of SEQ ID NO:35; amino acids 253 to 370 of SEQ ID NO:34; amino acids 268 to 386 of SEQ ID NO: 31; amino acids 269 to 387 of SEQ ID NO: 26, 29, 30 or 36; amino acids 269 to 397 of SEQ ID NO:25; amino acids 381 to 500 of SEQ ID NO: 24 or 27; and amino acids 401 to 520 of SEQ ID NO:28. In some embodiments, the binding region comprises at least one heavy chain variable (VH) domain comprising, consisting essentially of, or consisting of: amino acids 253 to 367 of SEQ ID NO:35; amino acids 253 to 370 of SEQ ID NO:34; amino acids 268 to 386 of SEQ ID NO:31; amino acids 269 to 387 of SEQ ID NO: 26, 29, 30 or 36; amino acids 269 to 397 of SEQ ID NO:25; amino acids 381 to 500 of SEQ ID NO: 24 or 27; and amino acids 401 to 520 of SEQ ID NO:28. In some embodiments, the binding region comprises at least one heavy chain variable (VH) domain comprising, consisting essentially of, or consisting of an amino acid sequence that is at least 85% (such as at least 90%, 91%, 92%, 93%, 94%, 95%, 96%, 97%, 98%, 99% or more) identical to the amino acid sequence shown in any one of: amino acids 269 to 387 of SEQ ID NO: 26, 29, 30 or 36; amino acids 269 to 397 of SEQ ID NO:25; amino acids 381 to 500 of SEQ ID NO: 24 or 27; and amino acids 401 to 520 of SEQ ID NO:28. In some embodiments, the binding region comprises at least one light chain variable (VL) domain comprising, consisting essentially of, or consisting of an amino acid sequence that is at least 85% (such as at least 90%, 91%, 92%, 93%, 94%, 95%, 96%, 97%, 98%, 99% or more) identical to the amino acid sequence shown in any one of: amino acids 269 to 375 of SEQ ID NO: 24, 27, or 28; amino acids 393 to 499 of SEQ ID NO:26; amino acids 403 to 513 of SEQ ID NO:25; amino acids 408 to 514 of SEQ ID NO:36; and amino acids 413 to 519 of SEQ ID NO: 29 or 30. In some embodiments, the binding region comprises at least one light chain variable (VL) domain comprising, consisting essentially of, or consisting of: amino acids 269 to 375 of SEQ ID NO: 24, 27, or 28; amino acids 393 to 499 of SEQ ID NO:26; amino acids 403 to 513 of SEQ ID NO:25; amino acids 408 to 514 of SEQ ID NO:36; and amino acids 413 to 519 of SEQ ID NO: 29 or 30. Any of heavy chain variable domain polypeptides described herein may be used in combination with any of the light chain variable domain polypeptides described herein.

In some embodiments, the binding region may comprise: (a) at least one heavy chain variable (VH) domain comprising, consisting essentially of, or consisting of: amino acids 269 to 387 of SEQ ID NOs: 26, 29, 30, or 36; amino acids 269 to 397 of SEQ ID NO:25; amino acids 381 to 500 of SEQ ID NO: 24 or 27; amino acids 401 to 522 of SEQ ID NO:36, or amino acids 401 to 520 of SEQ ID NO:28; and (b) at least one light chain variable (VL) domain comprising, consisting essentially of, or consisting of: amino acids 269 to 375 of SEQ ID NO: 24, 27, or 28; amino acids 393 to 499 of SEQ ID NO:26; amino acids 403 to 513 of SEQ ID NO:25; amino acids 408 to 514 of SEQ ID NO:36; and amino acids 413 to 519 of SEQ ID NO: 29 or 30. For example, the binding region may comprise (a) at least one heavy chain variable (VH) domain comprising, consisting essentially of, or consisting of amino acids 381 to 500 of SEQ ID NO:24; and (b) at least one light chain variable (VL) domain comprising, consisting essentially of, or consisting of amino acids 269 to 375 of SEQ ID NO:24. For example, the binding region may comprise (a) at least one heavy chain variable (VH) domain comprising, consisting essentially of, or consisting of amino acids 269 to 397 of SEQ ID NO:25; and (b) at least one light chain variable (VL) domain comprising, consisting essentially of, or consisting of: amino acids 403 to 513 of SEQ ID NO:25. For example, the binding region may comprise (a) at least one heavy chain variable (VH) domain comprising, consisting essentially of, or consisting of amino acids 269 to 387 of SEQ ID NO:26; and (b) at least one light chain variable (VL) domain comprising, consisting essentially of, or consisting of amino acids 393 to 499 of SEQ ID NO:26. For example, the binding region may comprise (a) at least one heavy chain variable (VH) domain comprising, consisting essentially of, or consisting of amino acids 381 to 500 of SEQ ID NO:27; and (b) at least one light chain variable (VL) domain comprising, consisting essentially of, or consisting of amino acids 269 to 375 of SEQ ID NO:27. For example, the binding region may comprise (a) at least one heavy chain variable (VH) domain comprising, consisting essentially of, or consisting of amino acids 401 to 520 of SEQ ID NO:28; and (b) at least one light chain variable (VL) domain comprising, consisting essentially of, or consisting of amino acids 269 to 375 of SEQ ID NO:28. For example, the binding region may comprise (a) at least one heavy chain variable (VH) domain comprising, consisting essentially of, or consisting of amino acids 269 to 387 of SEQ ID NO:29; and (b) at least one light chain variable (VL) domain comprising, consisting essentially of, or consisting of amino acids 413 to 519 of SEQ ID NO:29. For example, the binding region may comprise (a) at least one heavy chain variable (VH) domain comprising, consisting essentially of, or consisting of amino acids 269 to 387 of SEQ ID NO:30; and (b) at least one light chain variable (VL) domain comprising, consisting essentially of, or consisting of amino acids 413 to 519 of SEQ ID NO:30. For example, the binding region may comprise (a) at least one heavy chain variable (VH) domain comprising, consisting essentially of, or consisting of amino acids 269 to 387 of SEQ ID NO:36; and (b) at least one light chain variable (VL) domain comprising, consisting essentially of, or consisting of amino acids 408 to 514 of SEQ ID NO:36.

In some embodiments, the binding region comprises or consists essentially of amino acids 269-520 of SEQ ID NO: 102.

In some embodiments, the binding region comprises the heavy chain variable domain comprising or consisting essentially of amino acids 269 to 387 of SEQ ID NO:26, 29-30, or 36; 269 to 397 of SEQ ID NO:25; 381 to 500 of SEQ ID NO:27; or 401 to 522 of SEQ ID NO:36. In some embodiments, the binding region comprises the light chain variable domain comprising or consisting essentially of amino acids 269 to 375 of SEQ ID NO:27; 393 to 499 of SEQ ID NO:26; 403 to 513 of SEQ ID NO:25; 408 to 514 of SEQ ID NO:36; 413 to 519 of SEQ ID NO:29 or 30. In some embodiments, the binding region comprises or consists essentially of amino acids 269 to 513 of SEQ ID NO:25; 269 to 499 of SEQ ID NO:26; 269 to 519 of SEQ ID NO:29; 269 to 519 of SEQ ID NO:30; 268 to 386 of SEQ ID NO:31; 269 to 499 of SEQ ID NO:32; 269 to 499 of SEQ ID NO:33; 253 to 370 of SEQ ID NO:34; 253 to 367 of SEQ ID NO:35; or 269 to 514 of SEQ ID NO:36.

In some embodiments, a HER2 binding region is capable of specifically binding an extracellular part of human HER2. In some embodiments, the HER2 binding region comprises an immunoglobulin heavy chain variable region comprising: a CDR1 comprising the sequence of SEQ ID NO: 57; a CDR2 comprising the sequence of SEQ ID NO: 58; and a CDR3 comprising the sequence of SEQ ID NO: 59; and an immunoglobulin light chain variable region comprising: a CDR1 comprising the sequence of SEQ ID NO: 60; a CDR2 comprising the sequence of SEQ ID NO: 61; and a CDR3 comprising the sequence of SEQ ID NO: 62.

In some embodiments, a HER2 binding region comprises a sequence of SEQ ID NO: 224, as shown in Table 4 below. In some embodiments, the binding region has the sequence of SEQ ID NO: 224, or a sequence that is at least 95%, at least 96%, at least 97%, at least 98%, or at least 99% identical thereto. In some embodiments, the binding region has the sequence of SEQ ID NO: 224, or a sequence that has 1, 2, 3, 4, 5, 6, 7, 8, 9, 10, 11, 12, 13, 14, 15, 16, 17, 18, 19, 20, 21, 22, 23, 24, or 25 amino acid substitutions relative thereto.

TABLE 4
Exemplary HER2 binding region

| | SEQ ID | |
|---|---|---|
| Description | NO | Sequence |
| HER2 binding | 224 | QVQLQQSGPELKKPGETVKISCKASGYPFTNYGMNWVKQAPGQGLK |
| region | | WMGWINTSTGESTFADDFKGRFDFSLETSANTAYLQINNLKSEDSA |
| | | TYFCARWEVYHGYVPYWGQGTTVTVSSGGGGSGGGGSGGGGSGGGG |
| | | SGGGGSDIQMTQSPSSLSASVGDRVTITCKASQDVYNAVAWYQQKP |
| | | GQSPKLLIYSASSRYTGVPSRFTGSGSGPDFTFTISSVQAEDLAVY |
| | | FCQQHFRTPFTFGSGTKLEIK |

A natural ligand or derivative thereof may be utilized as the HER2 binding region for a cell targeting molecule. Native HER2 is known to heterodimerize with other members of the ErbB family upon binding ligands such as epidermal growth factors like epiregulin and heregulin. ErbB ligands which bind members of the ErbB family include EGF, TGF-beta, amphiregulin, betacellulin, HB-EGF, epiregulin, HER2-68 and HER2-100, heregulins, herstatin, NRG-2, NRG-3, and NRG-4. Examples of an ErbB ligand include the heregulins (HRG). Examples of heregulins include heregulin-a, heregulin-β1. heregulin-p2 and heregulin-p3; neu differentiation factor (NDF); acetylcholine receptor-inducing activity (ARIA); glial growth factors (GGFs); sensory and motor neuron derived factor (SMDF); γ-heregulin.

An ErbB ligand may also be a synthetic ErbB ligand. The synthetic ligand may be specific for a particular ErbB receptor or may recognize particular ErbB receptor complexes. An example of a synthetic ligand is the synthetic heregulin/EGF chimera biregulin and the EGF-like domain fragment HRG I 177-244. ErbB ligands or a part of an ErbB ligand that interacts with HER2 or a derivative thereof may be fused to Shiga toxin effector polypeptides to construct HER2-targeting, cell-targeting molecules that bind an extracellular part of HER2.

In some embodiments, small molecules which bind an extracellular part of HER2 may be utilized as the binding region for targeting. Many small molecules have been described which are capable of binding to HER2 such as tyrosine kinase inhibitors, AZD8931, lapatinib, neratinib (HKI-272), dacomitinib (PF-00299804), afatinib (BIBW 2992). Other small molecules which bind to an extracellular part of HER2 may be identified using methods well known to those of skill in the art, such as by derivatizing known EGFR binders like gefitinib, erlotinib, AEE788, AG1478, AG1571 (SU-5271), AP26113, CO-1686, XL647, vandetanib, and BMS-690514.

Any of the aforementioned HER2 binding molecules may be suitable for use as a HER2 binding region or modified to create one or more HER2 binding regions for use in a cell-targeting molecule as described herein.

### Shiga-Toxin Subunit Effector Polypeptides

Exemplary Shiga toxin effector polypeptides suitable for use in the binding molecules described herein are provided in Table 5. In some embodiments, the Shiga toxin effector polypeptide comprises the sequence of any one of SEQ ID NO: 1-21, 37, or 75-89. In some embodiments, the Shiga toxin effector polypeptide comprises a sequence that is at least 90%, at least 91%, at least 92%, at least 93%, at least 94%, at least 95%, at least 96% at least 97%, at least 98%, or at least 99% identical to any one of SEQ ID NO: 1-21, 37, or 75-89. In some embodiments, the Shiga toxin effector polypeptide comprises a sequence that has 1, 2, 3, 4, 5, 6, 7, 8, 9, 10, 11, 12, 13, 14, 15, 16, 17, 18, 19, 20, 21, 22, 23, 24, or 25, or more, amino acid substitutions relative to any one of SEQ ID NO: 1-21, 37, or 75-89. In some embodiments, the Shiga toxin effector polypeptide comprises the sequence of SEQ ID NO: 20, or a sequence that is at least 95%, at least 96%, at least 97%, at least 98%, or at least 99% identical thereto.

TABLE 5
Shiga Toxin Effectors

| | SEQ ID | |
|---|---|---|
| Description | NO: | Sequence |
| Shiga-like toxin 1 Subunit | 1 | KEFTLDFSTAKTYVDSLNVIRSAIGTPLQTISSGGTS |
| A (SLT-1A) | | LLMIDSGSGDNLFAVDVRGIDPEEGRFNNLRLIVERN |
| | | NLYVTGFVNRTNNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQIQRGFRTTLDDL |
| | | SGRSYVMTAEDVDLTLNWGRLSSVLPDYHGQDSVRVG |
| | | RISFGSINAILGSVALILNCHHHASRVARMASDEFPS |
| | | MCPADGRVRGITHNKILWDSSTLGAILMRRTISS |
| Shiga toxin Subunit A | 2 | KEFTLDFSTAKTYVDSLNVIRSAIGTPLQTISSGGTS |
| (StxA) | | LLMIDSGTGDNLFAVDVRGIDPEEGRFNNLRLIVERN |
| | | NLYVTGFVNRTNNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQIQRGFRTTLDDL |
| | | SGRSYVMTAEDVDLTLNWGRLSSVLPDYHGQDSVRVG |
| | | RISFGSINAILGSVALILNCHHHASRVARMASDEFPS |
| | | MCPADGRVRGITHNKILWDSSTLGAILMRRTISS |
| Shiga-like toxin 2 Subunit | 3 | DEFTVDFSSQKSYVDSLNSIRSAISTPLGNISQGGVS |
| A (SLT-2A) | | VSVINHVLGGNYISLNVRGLDPYSERFNHLRLIMERN |
| | | NLYVAGFINTETNIFYRFSDFSHISVPDVITVSMTTD |
| | | SSYSSLQRIADLERTGMQIGRHSLVGSYLDLMEFRGR |
| | | SMTRASSRAMLRFVTVIAEALRFRQIQRGFRPALSEA |
| | | SPLYTMTAQDVDLTLNWGRISNVLPEYRGEEGVRIGR |
| | | ISFNSLSAILGSVAVILNCHSTGSYSVRSVSQKQKTE |
| | | CQIVGDRAAIKVNNVLWEANTIAALLNRKPQDLTEPN |
| | | Q |
| Shiga toxin subtype c | 4 | KEFTLDFSTAKTYVDSLNVIRSAIGTPLQTISSGGTS |
| Subunit A (Stx1cA) | | LLMIDSGTGDNLFAVDVRGIDPEEGRFNNLRLIVERN |
| | | NLYVTGFVNRTNNVFYRFADFSHVTFPGTTAVTLSGD |
| | | SSYTTLQRVAGISRTGMQINRHSLTTSYLDLMSHSGT |
| | | SITQSVARAMLRFVTVTAEALRFRQIQRGFRTTLDDL |
| | | SGRSYVMTAEDVDLTLNWGRLSSVLPDYHGQDSVRVG |
| | | RISFGSVNAILGSVALILNCHHHASRVAR |
| Shiga toxin subtype d | 5 | KEFTLDFSTAKKYVDSLNVIRSAIGTPLQTISSGGTS |
| Subunit A (Stx1dA) | | LLMIDSGTGDNLFAVDIMGLEPEEERFNNLRLIVERN |
| | | NLYVTGFVNRTNNVFYRFADFSHVTFPGTRAVTLSGD |
| | | SSYTTLQRVAGISRTGMQINRHSLTTSYLDLMSYSGT |
| | | SLTQSVARAMLRFVTVTAEALRFRQIQRGFRTTLDDL |
| | | SGRSYVMTAEDVDLTLNWGRLSSILPDYHGQDSVRVG |
| | | RISFGSINAILGSVALILNCHHHASRVAR |
| Shiga toxin subtype e | 6 | QDFTVDFSTAKKYVDSLNAIRSAIGTPLHSISSGGTS |
| Subunit A (Stx1eA) | | LLMIDNGTGDNLFAVDIRGLDPEEERFDNLRLIIERN |
| | | NLYVTGFVNRTSNIFYRFADFSHVTFPGTRAVTLSGD |
| | | SSYTTLQRVAGIGRTGMQINRHSLTTSYLDLMSYSGS |
| | | SLTQPVARAMLRFVTVTAEALRFRQLQRGFRTTLDDV |
| | | SGHSYTMTVEDVDLTLNWGRLSSVLPDYHGQDSVRVG |
| | | RISFGGVNAILGSVALILNCHHHTSRVSR |
| Shiga toxin subtype 2c | 7 | REFTIDFSTQQSYVSSLNSIRTEISTPLEHISQGTTS |
| Subunit A (Stx2cA) variant | | VSVINHTPPGSYFAVDIRGLDVYQARFDHLRLIIEQN |
| 1 | | NLYVAGFVNTATNTFYRFSDFTHISVPGVTTVSMTTD |
| | | SSYTTLQRVAALERSGMQISRHSLVSSYLALMEFSGN |
| | | TMTRDASRAVLRFVTVTAEALRFRQIQREFRQALSET |
| | | APVYTMTPGDVDLTLNWGRISNVLPEYRGEDGVRVGR |
| | | ISFNNISAILGTVAVILNCHHQGARSVR |
| Shiga toxin subtype 2c | 8 | REFTIDFSTQQSYVSSLNSIRTEISTPLEHISQGITS |
| Subunit A (Stx2cA) variant | | VSVINHTPPGSYFAVDIRGLDVYQARFDHLRLIIEQN |
| 2 | | NLYVAGFVNTATNTFYRFSDFAHISVPGVTTVSMTTD |
| | | SSYTTLQRVAALERSGMQISRHSLVSSYLALMEFSGN |
| | | TMTRDASRAVLRFVTVTAEALRFRQIQREFRQALSET |
| | | APVYTMTPGDVDLTLNWGRISNVLPEYRGEDGVRVGR |
| | | ISFNNISAILGTVAVILNCHHQGARSVR |
| Shiga toxin subtype 2c | 9 | REFTIDFSTQQSYVSSINSIRTEISTPLEHISQGTTS |
| Subunit A (Stx2cA) variant | | VSVINHTPPGSYFAVDIRGLDIYQARFDHLRLIIEQN |
| 3 | | NLYVAGFVNTATNTFYRFSDFTHISVPGVTTVSMTTD |
| | | SSYTTLQRVAALERSGMQISRHSLVSSYLALMEFSGN |
| | | TMTRDASRAVLRFVTVTAEALRFRQIQREFRQALSET |
| | | APVYTMTPGDVDLTLNWGRISNVLPEYRGEDGVRVGR |
| | | ISFNNISAILGTVAVILNCHHQGARSVR |
| Shiga toxin subtype 2c | 10 | REFTIDESTQQSYVSSLNSIRTEISTPLEHISQGTTS |
| Subunit A (Stx2cA) variant | | VSVINHTPPGSYFAVDIRGLDVYQARFDHLRLIIEQN |
| 4 | | NLYVAGFVNTATNTFYRFSDFTHISVPSVTTVSMTTD |
| | | SSYTTLQRVAALERSGMQISRHSLVSSYLALMEFSGN |
| | | TMTRDASRAVLRFVTVTAEALRFRQIQREFRQALSET |
| | | APVYTMTPGDVDLTLNWGRISNVLPEYRGEDGVRVGR |
| | | ISFNNISAILGTVAVILNCHHQGARSVR |
| Shiga toxin subtype 2c | 11 | REFTIDFSTQQSYVSSLNSIRTEISTPLEHISQGTTS |
| Subunit A (Stx2cA) variant | | VSVINHTPPGSYFAVDIRGLDVYQARFDHLRLIIEQN |
| 5 | | NLYMAGFVNTATNTFYRFSDFTHISVPSVTTVSMTTD |
| | | SSYTTLQRVAALERSGMQISRHSLVSSYLALMEFSGN |
| | | TMTRDASRAVLRFVTVTAEALRFRQIQREFRQALSET |
| | | APVYTMTPGDVDLTLNWGRISNVLPEYRGEDGVRVGR |
| | | ISFNNISAILGTVAVILNCHHQGARSVR |
| Shiga toxin subtype 2c | 12 | REFTIDFSTQQSYVSSLNSIRTEISTPLEHISQGTTS |
| Subunit A (Stx2cA) variant | | VSVINHTPPGSYFAVDIRGLDVYQARFDHLRLIIEQN |
| 6 | | NLYVAGFVNTATNTFYRFSDFTHISVPGVTTVSMTTD |
| | | SSYTTLQRVAALERSGMQISRHSLVSSYLALMEFSGN |
| | | TMTRDASRAVLRFVTVTAEALRFRQIQREFRQVLSET |
| | | APVYTMTPGDVDLTLNWGRISNVLPEYRGEDGVRVGR |
| | | ISFNNISAILSTVAVILNCHHQGARSVR |
| Shiga toxin subtype 2d | 13 | REFTIDFSTQQSYVSSLNSIRTEISTPLEHISQGTTS |
| Subunit A (Stx2dA) variant | | VSVINHTPPGSYFAVDIRGLDVYQARFDHLRLIIEQN |
| 1 | | NLYVAGFVNTATNTFYRFSDFAHISVPGVTTVSMTTD |
| | | SSYTTLQRVAALERSGMQISRHSLVSSYLALMEFSGN |
| | | TMTRDASRAVLRFVTVTAEALRFRQIQREFRQALSET |
| | | APVYTMTPGDVDLTLNWGRISNVIPEYRGEDGVRVGR |
| | | ISFNNISAILGTVAVILNCHHQGARSVR |
| Shiga toxin subtype 2d | 14 | REFMIDFSTQQSYVSSLNSIRTEISTPLEHISQGTTS |
| Subunit A (Stx2dA) variant | | VSVINHTPPGSYFAVDIRGLDVYQARFDHLRLIIEQN |
| 2 | | NLYVAGFVNTATNTFYRFSDFTHISVPGVTTVSMTTD |
| | | SSYTTLQRVAALERSGMQISRHSLVSSYLALMEFSGN |
| | | TMTRDASRAVLRFVTVTAEALRFRQIQREFRQALSET |
| | | APVYTMTPEEVDLTLNWGRISNVLPEFRGEGGVRVGR |
| | | ISFNNISAILGTVAVILNCHHQGARSVR |
| Shiga toxin subtype 2d | 15 | REFTIDFSTQQSYVSSLNSIRTEISTPLEHISQGTTS |
| Subunit A (Stx2dA) variant | | VSVINHTPPGSYFAVDIRGLDVYQARFDHLRLIIEQN |
| 3 | | NLYVAGFVNTATNTFYRFSDFTHISVPGVTTVSMTTD |
| | | SSYTTLQRVAALERSGMQISRHSLVSSYLALMEFSGN |
| | | TMTRDASRAVLRFVTVTAEALRFRQIQREFRQALSET |
| | | APVYTMTPGDVDLTLNWGRISNVIPEYRGEDGVRVGR |
| | | ISFNNISAILSTVAVILNCHHQGARSVR |
| Shiga toxin subtype 2e | 16 | QEFTIDFSTQQSYVSSLNSIRTAISTPLEHISQGATS |
| Subunit A (Stx2eA) variant | | VSVINHTPPGSYISVGIRGLDVYQERFDHLRLIIERN |
| 1 | | NLYVAGFVNTTTNTFYRFSDFAHISLPGVTTISMTTD |
| | | SSYTTLQRVAALERSGMQISRHSLVSSYLALMEFSGN |
| | | TMTRDASRAVLRFVTVTAEALRFRQIQREFRLALSET |
| | | APVYTMTPEDVDLTLNWGRISNVLPEYRGEAGVRVGR |
| | | ISFNNISAILGTVAVILNCHHQGARSVR |
| Shiga toxin subtype 2e | 17 | QEFTIDFSTQQSYVSSLNSIRTAISTPLEHISQGATS |
| Subunit A (Stx2eA) variant | | VSVINHTPPGSYISVGIRGLDVYQAHFDHLRLIIEQN |
| 2 | | NLYVAGFVNTATNTFYRFSDFAHISLPGVTTISMTTD |
| | | SSYTTLQRVAALERSGMQISRHSLVSSYLALMEFSGN |
| | | TMTREASRAVLRFVTVTAEALRFRQIQREFRQALSET |
| | | APVYTMTPEDVDLTLNWGRISNVLPEYRGEDGVRVGR |
| | | ISFNNISAILGTVAVILNCHHQGARSVR |
| Shiga toxin subtype 2f | 18 | DEFTVDFSSQKSYVDSLNSIRSAISTPLGNISQGGVS |
| Subunit A (Stx2fA) | | VSVINHVPGGNYISLNVRGLDPYSERFNHLRLIMERN |
| | | NLYVAGFINTETNTFYRFSDFSHISVPDVITVSMTTD |
| | | SSYSSLQRIADLERTGMQIGRHSLVGSYLDLMEFRGR |
| | | SMTRASSRAMLRFVTVIAEALRFRQIQRGFRPALSEA |
| | | SPLYTMTAQDVDLTLNWGRISNVLPEYRGEEGVRIGR |
| | | ISFNSLSAILGSVAVILNCHSTGSYSVR |
| SLTA-DI-1 | 19 | AEFTLDFSTAKTYVDSLNVIRSAIGTPLQTISSGGTS |
| | | LLMIDSGIGDNLFAVDILGFDFTLGRFNNLRLIVERN |
| | | NLYVTGFVNRTNNVFYRFADFSHVTFPGTTAVTLSAD |
| | | SSYTTLQRVAGISRIGMQINRHSITTSYLDLMSHSAT |
| | | SLTQSVARAMLRFVTVTAEALRFRQIQRGFRTTLDDL |
| | | SGRSYVMTAEDVDLTLNWGRLSSVLPDYHGQDSVRVG |
| | | RISFGSINAILGSVALILNSHHHASAVAA |
| SLTA-DI-2 | 20 | KEFTLDFSTAKTYVDSLNVIRSAIGTPLQTISSGGTS |
| | | LLMIDSGIGDNLFAVDILGFDFTLGRFNNLRLIVERN |
| | | NLYVTGFVNRTNNVFYRFADFSHVTFPGTTAVTLSAD |
| | | SSYTTLQRVAGISRTGMQINRHSLTTSYLDLMSHSGT |
| | | SLTQSVARAMLRFVTVTAEALRFRQIQRGFRTTLDDL |
| | | SGASYVMTAEDVDLTLNWGRLSSVLPDYHGQDSVRVG |
| | | RISEGSINAILGSVALILNSHHHASAVAA |
| SLTA-DI-3 | 21 | REFTLDFSTARTYVDSLNVIRSAIGTPLQTISSGGTS |
| | | LLMIDSGIGDNLFAVDILGFDFTLGRFNNLRLIVERN |
| | | NLYVTGFVNRTNNVFYRFADFSHVTFPGTTAVTLSAD |
| | | SSYTTLQRVAGISRIGMQINRHSITTSYLALMSHSGT |
| | | SLTQSVARAMLRFVTVTAEALRFRQIQRGFRTTLDDL |
| | | SGASYVMTAEDVDLTLNWGRLSSVLPDYHGQDSVRVG |
| | | RISFGSINAILGSVALILNSHHHASAVAA |
| STLA-FR | 37 | KEFTLDFSTAKTYVDSLNVIRSAIGTPLQTISSGGTS |
| | | LLMIDSGSGDNLFAVDVRGIDPEEGRFNNLRLIVERN |
| | | NLYVTGFVNRTNNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQIQRGFRTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 1 | 75 | KEFILRFSVAHKYVDSLNVIRSAIGTPLQTISSGGTS |
| | | LLMIDSGSGDNLFAVDVRGIDPEEGRFNNLRLIVERN |
| | | NLYVTGFVNRINNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQIQRGERTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 2 | 76 | KEFTLDFSTAKTYVDSLNVIRSAIGTPLQTISSGGTS |
| | | LLMIDNLVPMVATVVDVRGIDPEEGRFNNLRLIVERN |
| | | NLYVTGFVNRTNNVFYRFADFSHVTFPGTTAVTLSGD |
| | | SSYTTLQRVAGISRTGMQINRHSLTTSYLDLMSHSGT |
| | | SITQSVARAMLRFVTVTAEALRFRQIQRGERTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 3 | 77 | KEFTLDFSTAKTYVDSLNVIRSAIGTPLQTISSGGTS |
| | | LLMIDSNLVPMVATVDVRGIDPEEGRFNNLRLIVERN |
| | | NLYVTGFVNRTNNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQLQRGFRTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 4 | 78 | KEFTLDFSTAKTYVDSLNVIRSAIGTPLQTISSGGTS |
| | | LLMIDSGILGFVFTLDVRGIDPEEGRFNNLRLIVERN |
| | | NLYVTGFVNRTNNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQIQRGFRTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 5 | 79 | KEFTLDFSTAKTYVDSLNVIRSAIGTPLQTISSGGTS |
| | | LLMIDSGSGDNLFAVGILGFDFTLGRFNNLRLIVERN |
| | | NLYVTGFVNRINNVFYRFADFSHVTFPGTTAVTLSGD |
| | | SSYTTLQRVAGISRIGMQINRHSLTTSYLDLMSHSGT |
| | | SLTQSVARAMLRFVTVTAEALRFRQIQRGFRTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 6 | 80 | KEFTLDFSTAKTYVDSLNVIRSAIGTPLQTISSGGTS |
| | | LLMIDSGSGDNLFAVDILGFVFTLGRFNNLRLIVERN |
| | | NLYVTGFVNRTNNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQIQRGFRTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 7 | 81 | KEFTLDFSTAKTYVDSLNVIRSAIGTPLQTISSGGTS |
| | | LLMIDSGSGDNLFAVDILGFDFTLGRFNNLRLIVERN |
| | | NLYVTGFVNRTNNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQIQRGERTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 8 | 82 | KEFTLDFSTAKTYVDSLNVIRSAIGTPLQTISSGGTS |
| | | LLMIDSGSGDNLFAVGILGFVFTLGRFNNLRLIVERN |
| | | NLYVTGFVNRINNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQIQRGERTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 9 | 83 | KEFILDFSTAKTYVDSLNVIRSAIGTPLQTISSGGTS |
| | | LLMIDSGIGDNLFAVDVRGIAPIEARFNNLRLIVERN |
| | | NLYVTGFVNRTNNVFYRFADFSHVTFPGTTAVTLSAD |
| | | SSYTTLQRVAGISRTGMQINRHSLTTSYLDLMSHSAT |
| | | SLTQSVARAMLRFVTVTAEALRFRQIQRGFRTTLAAL |
| | | SGASYVMTAEDVDLTLNWGRLSSVLPDYHGQDSVRVG |
| | | RISFGSINAILGSVALILNSHHHASAVAA |
| SLT-1A-combo variant 10 | 84 | KEFTLDFSTAKTYVDSLNVIRSAIGTPLQTISSGGTS |
| | | LLMIDSGSGDNLFAVGILGFVFTLEGRFNNLRLIVER |
| | | NNLYVTGFVNRTNNVFYRFADFSHVTFPGTTAVTLSG |
| | | DSSYTTLQRVAGISRTGMQINRHSLTTSYLDLMSHSG |
| | | TSLTQSVARAMLRFVTVTAEALRFRQIQRGFRTTLDD |
| | | LSGRSYVMTAEDVDLTLNWGRLSSVLPDYHGQDSVRV |
| | | GRISFGSINAILGSVALILNCHHHASAVAA |
| SLT-1A-combo variant 11 | 85 | KEFTLDFSTAKTYVDSLNVIRSAIGTPLQTISSGGTS |
| | | LLMIDSGSGDNLFAVNLVPMVATVGRFNNLRLIVERN |
| | | NLYVTGFVNRTNNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQIQRGFRTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 12 | 86 | KEFTLDFSTAKTYVDSLNVIRSAIGTPLQTISSGGTS |
| | | LLMIDSGSGDNLFAVDVRGIDPEEGRFNNLRLIVERN |
| | | NLYVTGFVNRTNNVFYRFADFSHVTFPGTNLVPMVAT |
| | | VSYTTLQRVAGISRTGMQINRHSLTTSYLDLMSHSGT |
| | | SLTQSVARAMLRFVTVTAEALRFRQIQRGFRTTLDDL |
| | | SGRSYVMTAEDVDLTLNWGRLSSVLPDYHGQDSVRVG |
| | | RISFGSINAILGSVALILNCHHHASAVAA |
| SLT-1A-combo variant 13 | 87 | KEFTLDFSTAKTYVDSLNVIRSAIGTPLQTISSGGTS |
| | | LLMIDSGSGDNLFAVDVRGIDPEEGRFNNLRLIVERN |
| | | NLYVTGFVNRINNVFYRFADFSHVTFPGTTAVTLSGD |
| | | SSYTTLQRVAGISRTGMQINRHSLTTSYLDLMSHSGT |
| | | SLTQSVARAMLRFVIVTAEALRFRQIQRGFRGILGDV |
| | | FTLSYVMTAEDVDLTLNWGRLSSVLPDYHGQDSVRVG |
| | | RISFGSINAILGSVALILNSHHHASAVAA |
| SLT-1A-combo variant 14 | 88 | KEFTLDFSTAKTYVDSLNVIRSAIGTPLQTISSGGTS |
| | | LLMIDSGSGDNLFAVDVRGIDPEEGRFNNLRLIVERN |
| | | NLYVTGFVNRINNVFYRFADFSHVTFPGTTAVTLSGD |
| | | SSYTTLQRVAGISRTGMQINRHSLTTSYLDLMSHSGT |
| | | SITQSVARAMLRFVTVTAEALRFRQIQRGERTTLDDI |
| | | SGRSYVMTAEDVDLTLNWGRLSSVLPDYHGQDSVRVG |
| | | RISFGSINAILGSVALILNCHHHILRFSVAHKASAVA |
| | | A |
| SLT-1A-combo variant 15 | 89 | KEFTLDFSTAKTYVDSLNVIRSAIGTPLQTISSGGTS |
| | | LLMIDSGSGDNLFAVDILGFDFTLGRFNNLRLIVERN |
| | | NLYVTGFVNRINNVFYRFADFSHVTFPGTTAVTLSGD |
| | | SSYTTLQRVAGISRTGMQINRHSLTTSYLDLMSHSGT |
| | | SLTQSVARAMLRFVTVTAEALRFRQIQRGFRTTLDDL |
| | | SGRSYVMTAEDVDLTLNWGRLSSVLPDYHGQDSVRVG |
| | | RISFGSINAILGSVALILNCHHHARNLVPMVATVASA |
| | | VAA |

In some embodiments, the Shiga toxin effector polypeptides described herein are de-immunized, such as, e.g., as compared to a wild-type Shiga toxin, wild-type Shiga toxin polypeptide, and/or Shiga toxin effector polypeptide comprising only wild-type polypeptide sequences. The de-immunized, Shiga toxin effector polypeptides each comprise a disruption of at least one (such as, e.g., at least two, three, four, five, six, seven, eight, nine or more), putative, endogenous, epitope region in order to reduce the antigenic and/or immunogenic potential of the Shiga toxin effector polypeptide after administration of the polypeptide to a subject. A Shiga toxin effector polypeptide and/or Shiga toxin A Subunit polypeptide, whether naturally occurring or not, can be de-immunized by a method described herein and/or known to the skilled worker, wherein the resulting molecule retains or exhibits one or more Shiga toxin A Subunit functions.

In some embodiments, the Shiga toxin effector polypeptides described herein comprise a disruption of an endogenous epitope or epitope region, such as, e.g., a B-cell and/or CD4+ T-cell epitope. In some embodiments, the Shiga toxin effector polypeptide comprises a disruption of at least one (such as at least two, three, four, five, six, seven, eight or more) endogenous, B-cell and/or CD4+ T-cell epitope region. In some embodiments, the Shiga toxin effector polypeptide comprises a disruption of at least one (such as at least two, three, four, five, six, seven, eight or more), endogenous, epitope region described herein, wherein the disruption reduces the antigenic and/or immunogenic potential of the Shiga toxin effector polypeptide after administration of the polypeptide to a subject, and wherein the Shiga toxin effector polypeptide is capable of exhibiting one or more Shiga toxin A Subunit functions, such as, e.g., a significant level of Shiga toxin cytotoxicity. For example, the Shiga toxin effector polypeptide may comprise a disruption of at least three, endogenous, B-cell and/or CD4+ T-cell epitope regions (such as, e.g., due to two or more mutations and one or more truncations relative to a wild-type Shiga toxin A Subunit).

The term “disrupted” or “disruption” as used herein with regard to an epitope region refers to the deletion of at least one (such as at least two, three, four, five, six, seven, eight or more) amino acid residue in an epitope region, inversion of two or more amino acid residues where at least one of the inverted amino acid residues is in an epitope region, insertion of at least one (such as at least two, three, four, five, six, seven, eight or more) amino acid into an epitope region, and a substitution of at least one amino acid residue in an epitope region. An epitope region disruption by mutation includes amino acid substitutions with non-standard amino acids and/or non-natural amino acids. Epitope regions may alternatively be disrupted by mutations comprising the modification of an amino acid by the addition of a covalently-linked chemical structure which masks at least one amino acid in an epitope region, such as PEGylation, small molecule adjuvants, and site-specific albumination.

In some embodiments, the de-immunized, Shiga toxin effector polypeptides comprise a disruption of at least one (such as at least two, three, four, five, six, seven, eight or more) epitope region provided herein. For example, the de-immunized, Shiga toxin effector polypeptide may comprise a disruption of at least three epitope regions provided herein. In some embodiments, the de-immunized, Shiga toxin effector polypeptide comprises a disruption of at least four epitope regions provided herein. In some embodiments, the de-immunized, Shiga toxin effector polypeptide comprises a disruption of at least five epitope regions provided herein. As described herein, when the Shiga toxin effector polypeptide also comprises an embedded or inserted, heterologous, CD8+ T-cell epitope, at least some number of disrupted, endogenous, B-cell and/or CD4+ T-cell epitope region does not overlap with the embedded or inserted, heterologous, CD8+ T-cell epitope.

In some embodiments, the de-immunized, Shiga toxin effector polypeptide comprises, consists of, or consists essentially of a full-length Shiga toxin A Subunit (e.g. SLT-1A (SEQ ID NO: 1), StxA (SEQ ID NO:2), or SLT-2A (SEQ ID NO:3)) comprising at least one disruption of the amino acid sequence selected from the group of natively positioned amino acids consisting of: 1-15 of SEQ ID NO: 1 or SEQ ID NO:2; 3-14 of SEQ ID NO:3; 26-37 of SEQ ID NO:3; 27-37 of SEQ ID NO: 1 or SEQ ID NO:2; 39-48 of SEQ ID NO: 1 or SEQ ID NO:2; 42-48 of SEQ ID NO:3; 53-66 of SEQ ID NO: 1, SEQ ID NO:2, or SEQ ID NO:3; 94-115 of SEQ ID NO: 1, SEQ ID NO:2, or SEQ ID NO:3; 141-153 of SEQ ID NO: I or SEQ ID NO:2; 140-156 of SEQ ID NO:3; 179-190 of SEQ ID NO: 1 or SEQ ID NO:2; 179-191 of SEQ ID NO:3; 204 of SEQ ID NO:3; 205 of SEQ ID NO:1 or SEQ ID NO:2; 210-218 of SEQ ID NO:3; 240-258 of SEQ ID NO:3; 243-257 of SEQ ID NO: 1 or SEQ ID NO:2; 254-268 of SEQ ID NO: 1 or SEQ ID NO:2; 262-278 of SEQ ID NO:3; 281-297 of SEQ ID NO:3; and 285-293 of SEQ ID NO: 1 or SEQ ID NO:2, or the equivalent position in a Shiga toxin A Subunit polypeptide, conserved Shiga toxin effector polypeptide sub-region, and/or non-native, Shiga toxin effector polypeptide sequence (such as the Shiga toxin effector polypeptides shown in SEQ ID NOs: 4-18).

In some embodiments, the de-immunized Shiga toxin effector polypeptide comprises, consists essentially of, or consists of a full-length or truncated Shiga toxin A Subunit (e.g. SLT-1A (SEQ ID NO: 1), StxA (SEQ ID NO:2), SLT-2A (SEQ ID NO:3), or any one of SEQ ID NOs: 7-18 further comprising a disruption of at least one (such as at least two, three, four, five, six, seven, eight or more) endogenous, B-cell and/or CD4+ T-cell epitope region, wherein the B-cell region is selected from the group of natively positioned Shiga toxin A Subunit regions consisting of: 1-15 of SEQ ID NO: 1 or SEQ ID NO:2; 3-14 of SEQ ID NO:3; 26-37 of SEQ ID NO:3; 27-37 of SEQ ID NO: 1 or SEQ ID NO:2; 39-48 of SEQ ID NO: 1 or SEQ ID NO:2; 42-48 of SEQ ID NO:3; 53-66 of SEQ ID NO: 1, SEQ ID NO:2, or SEQ ID NO:3; 94-1 15 of SEQ ID NO: 1, SEQ ID NO:2, or SEQ ID NO:3; 141-153 of SEQ ID NO: I or SEQ ID NO:2; 140-156 of SEQ ID NO:3; 179-190 of SEQ ID NO: 1 or SEQ ID NO:2; 179-191 of SEQ ID NO:3; 204 of SEQ ID NO:3; 205 of SEQ ID NO: 1 or SEQ ID NO:2, and 210-218 of SEQ ID NO:3; 240-260 of SEQ ID NO:3; 243-257 of SEQ ID NO: 1 or SEQ ID NO:2; 254-268 of SEQ ID NO: 1 or SEQ ID NO:2; 262-278 of SEQ ID NO:3; 281-297 of SEQ ID NO:3; and 285-293 of SEQ ID NO: 1 or SEQ ID NO:2; or the equivalent region in a Shiga toxin A Subunit or derivative thereof (such as the equivalent region in any one of the Shiga toxin 1 A Subunit variants shown in SEQ ID NOs: 4-6 and the Shiga-like toxin 2 A Subunit variants shown in SEQ ID NOs: 7-18); and the CD4+ T-cell epitope region is selected from the group of natively positioned Shiga toxin A Subunit regions consisting of: 4-33 of SEQ ID NO: 1 or SEQ ID NO:2; 34-78 of SEQ ID NO: 1 or SEQ ID NO:2; 77-103 of SEQ ID NO: 1 or SEQ ID NO:2; 128-168 of SEQ ID NO: 1 or SEQ ID NO:2; 160-183 of SEQ ID NO: 1 or SEQ ID NO:2; 236-258 of SEQ ID NO: 1 or SEQ ID NO:2; and 274-293 of SEQ ID NO: 1 or SEQ ID NO:2; or the equivalent region in a Shiga toxin A Subunit or derivative thereof (such as the equivalent region in any one of the Shiga toxin 1 A Subunit variants shown in SEQ ID NOs: 4-6 and the Shiga-like toxin 2 A Subunit variants shown in SEQ ID NOs: 7-18). In some embodiments, the B-cell epitope region is selected from the group of natively positioned Shiga toxin A Subunit regions consisting of: 1-15 of SEQ ID NO: 1 or SEQ ID NO:2; 3-14 of SEQ ID NO:3; 26-37 of SEQ ID NO:3; 27-37 of SEQ ID NO: 1 or SEQ ID NO:2; 39-48 of SEQ ID NO: 1 or SEQ ID NO:2; 42-48 of SEQ ID NO:3; 53-66 of SEQ ID NO: 1, SEQ ID NO:2, or SEQ ID NO:3; 94-1 15 of SEQ ID NO: 1, SEQ ID NO:2, or SEQ ID NO:3; 141-153 of SEQ ID NO: 1 or SEQ ID NO:2; 140-156 of SEQ ID NO:3; 179-190 of SEQ ID NO: 1 or SEQ ID NO:2; 179-191 of SEQ ID NO:3; 204 of SEQ ID NO:3; 205 of SEQ ID NO: 1 or SEQ ID NO:2; 210-218 of SEQ ID NO:3 and 243-257 of SEQ ID NO: 1 or SEQ ID NO:2; or the equivalent region in a Shiga toxin A Subunit or derivative thereof (such as the equivalent region in any one of the Shiga toxin 1 A Subunit variants shown in SEQ ID NOs: 4-6 and the Shiga-like toxin 2 A Subunit variants shown in SEQ ID NOs: 7-18); and the CD4+ T-cell epitope region is selected from the group of natively positioned Shiga toxin A Subunit regions consisting of: 4-33 of SEQ ID NO: 1 or SEQ ID NO:2; 34-78 of SEQ ID NO: 1 or SEQ ID NO:2; 77-103 of SEQ ID NO: 1 or SEQ ID NO:2; 128-168 of SEQ ID NO: 1 or SEQ ID NO:2; 160-183 of SEQ ID NO: 1 or SEQ ID NO:2; and 236-258 of SEQ ID NO: 1 or SEQ ID NO:2; or the equivalent region in a Shiga toxin A Subunit or derivative thereof (such as the equivalent region in any one of the Shiga toxin 1 A Subunit variants shown in SEQ ID NOs: 4-6 and the Shiga-like toxin 2 A Subunit variants shown in SEQ ID NOs: 7-18).

In some embodiments, the de-immunized Shiga toxin effector polypeptide comprises, consists essentially of, or consists of a full-length or truncated Shiga toxin A Subunit (e.g. SLT-1A (SEQ ID NO: 1), StxA (SEQ ID NO:2), Shiga toxin 1 A Subunit variant effector polypeptide (SEQ ID NOs: 4-6), SLT-2A (SEQ ID NO:3), or Shiga-like toxin 2 A Subunit variant effector polypeptide (SEQ ID NOs: 7-18)) comprising a disruption of at least three, endogenous, B-cell and/or CD4+ T-cell epitope regions, wherein the disruption comprises a mutation, relative to a wild-type Shiga toxin A Subunit, in the B-cell epitope region selected from the group of natively positioned Shiga toxin A Subunit regions consisting of: 1-15 of SEQ ID NO: 1 or SEQ ID NO:2; 3-14 of SEQ ID NO:3; 26-37 of SEQ ID NO:3; 27-37 of SEQ ID NO: 1 or SEQ ID NO:2; 39-48 of SEQ ID NO: 1 or SEQ ID NO:2; 42-48 of SEQ ID NO:3; 53-66 of SEQ ID NO: 1, SEQ ID NO:2, or SEQ ID NO:3; 94-1 15 of SEQ ID NO: 1, SEQ ID NO:2, or SEQ ID NO:3; 141-153 of SEQ ID NO: 1 or SEQ ID NO:2; 140-156 of SEQ ID NO:3; 179-190 of SEQ ID NO: 1 or SEQ ID NO:2; 179-191 of SEQ ID NO:3; 204 of SEQ ID NO:3; 205 of SEQ ID NO: 1 or SEQ ID NO:2; 210-218 of SEQ ID NO:3 and 243-257 of SEQ ID NO: 1 or SEQ ID NO:2; or the equivalent region in a Shiga toxin A Subunit or derivative thereof (such as the equivalent region in any one of the Shiga toxin 1 A Subunit variants shown in SEQ ID NOs: 4-6 and Shiga-like toxin 2 A Subunit variants shown in SEQ ID NOs: 7-18); and/or the CD4+ T-cell epitope region selected from the group of natively positioned Shiga toxin A Subunit regions consisting of: 4-33 of SEQ ID NO: 1 or SEQ ID NO:2; 34-78 of SEQ ID NO: 1 or SEQ ID NO:2; 77-103 of SEQ ID NO: 1 or SEQ ID NO:2; 128-168 of SEQ ID NO: 1 or SEQ ID NO:2; 160-183 of SEQ ID NO: 1 or SEQ ID NO:2; and 236-258 of SEQ ID NO: 1 or SEQ ID NO:2; or the equivalent region in a Shiga toxin A Subunit or derivative thereof (such as the equivalent region in any one of the Shiga toxin 1 A Subunit variants shown in SEQ ID NOs: 4-6 and the Shiga-like toxin 2 A Subunit variants shown in SEQ ID NOs: 7-18). In some embodiments, each of the at least three of the B-cell and/or CD4+ T-cell epitope regions comprises a disruption comprising an amino acid residue substitution relative to a wild-type Shiga toxin A Subunit sequence.

In some embodiments, the Shiga toxin effector polypeptide comprises, consists of, or consists essentially of a truncated Shiga toxin A Subunit. Truncations of Shiga toxin A Subunits might result in the deletion of an entire epitope region(s) without affecting Shiga toxin effector function(s). The smallest Shiga toxin A Subunit fragment shown to exhibit significant enzymatic activity was a polypeptide composed of residues 75-247 of StxA (Al-Jaufy A et al, Infect Immun 62: 956-60 (1994)). Truncating the carboxy-terminus of SLT-1A, StxA, or SLT-2A to amino acids 1-25 1 removes two predicted B-cell epitope regions, two predicted CD4 positive (CD4+) T-cell epitopes, and a predicted, discontinuous, B-cell epitope. Truncating the amino-terminus of SLT-1A, StxA, or SLT-2A to 75-293 removes at least three, predicted, B-cell epitope regions and three predicted CD4+ T-cell epitopes. Truncating both amino- and carboxy-terminals of SLT-1A, StxA, or SLT-2A to 75-251 deletes at least five, predicted, B-cell epitope regions; four, putative, CD4+ T-cell epitopes; and one, predicted, discontinuous, B-cell epitope.

In some embodiments, a Shiga toxin effector polypeptide may comprise, consist of, or consist essentially of a full-length or truncated Shiga toxin A Subunit with at least one (such as at least two, three, four, five, six, seven, eight or more) mutation, e.g. deletion, insertion, inversion, or substitution, in a provided epitope region. In some embodiments, the polypeptides comprise a disruption which comprises a deletion of at least one amino acid within the epitope region. In some embodiments, the polypeptides comprise a disruption which comprises an insertion of at least one amino acid within the epitope region. In some embodiments, the polypeptides comprise a disruption which comprises an inversion of amino acids, wherein at least one inverted amino acid is within the epitope region. In some embodiments, the polypeptides comprise a disruption which comprises a substitution of at least one (such as at least two, three, four, five, six, seven, eight or more) amino acid within the epitope region. In some embodiments, the polypeptides comprise a disruption which comprises a mutation, such as an amino acid substitution to a non-standard amino acid or an amino acid with a chemically modified side chain.

In some embodiments, the Shiga toxin effector polypeptides may comprise, consist of, or consist essentially of a full-length or truncated Shiga toxin A Subunit with one or more mutations as compared to the native sequence which comprises at least one amino acid substitution selected from the group consisting of: A, G, V, L, I, P, C, M, F, S, D, N, Q, H, and K. In some embodiments, the polypeptide may comprise, consist of, or consist essentially of a full-length or truncated Shiga toxin A Subunit with a single mutation as compared to the native sequence wherein the substitution is selected from the group consisting of: D to A, D to G, D to V, D to L, D to I, D to F, D to S, D to Q, E to A, E to G, E to V, E to L, E to I, E to F, E to S, E to Q, E to N, E to D, E to M, E to R, G to A, H to A, H to G, H to V, H to L, H to I, H to F, H to M, K to A, K to G, K to V, K to L, K to I, K to M, K to H, L to A, L to G, N to A, N to G, N to V, N to L, N to I, N to F, P to A, P to G, P to F, R to A, R to G, R to V, R to L, R to I, R to F, R to M, R to Q, R to S, R to K, R to H, S to A, S to G, S to V, S to L, S to I, S to F, S to M, T to A, T to G, T to V, T to L, T to I, T to F, T to M, T to S, Y to A, Y to G, Y to V, Y to L, Y to I, Y to F, and Y to M.

In some embodiments, the Shiga toxin effector polypeptides comprise, consist of, or consist essentially of a full-length or truncated Shiga toxin A Subunit with one or more mutations as compared to the native amino acid residue sequence which comprises at least one amino acid substitution of an immunogenic residue and/or within an epitope region, wherein at least one substitution occurs at the natively positioned group of amino acids selected from the group consisting of: 1 of SEQ ID NO: 1 or SEQ ID NO:2; 4 of SEQ ID NO: 1, SEQ ID NO:2, or SEQ ID NO:3; 8 of SEQ ID NO: 1, SEQ ID NO:2, or SEQ ID NO:3; 9 of SEQ ID NO: 1, SEQ ID NO:2, or SEQ ID NO:3; 11 of SEQ ID NO: 1, SEQ ID NO:2, or SEQ ID NO:3; 33 of SEQ ID NO: 1 or SEQ ID NO:2; 43 of SEQ ID NO: 1 or SEQ ID NO:2; 44 of SEQ ID NO: 1 or SEQ ID NO:2; 45 of SEQ ID NO: 1 or SEQ ID NO:2; 46 of SEQ ID NO: 1 or SEQ ID NO:2; 47 of SEQ ID NO: 1 or SEQ ID NO:2; 48 of SEQ ID NO: 1, SEQ ID NO:2, or SEQ ID NO:3; 49 of SEQ ID NO: 1 or SEQ ID NO:2; 50 of SEQ ID NO: 1 or SEQ ID NO:2; 5 1 of SEQ ID NO: 1 or SEQ ID NO:2; 53 of SEQ ID NO: 1 or SEQ ID NO:2; 54 of SEQ ID NO: 1 or SEQ ID NO:2; 55 of SEQ ID NO: 1 or SEQ ID NO:2; 56 of SEQ ID NO: 1 or SEQ ID NO:2; 57 of SEQ ID NO: 1 or SEQ ID NO:2; 58 of SEQ ID NO: 1, SEQ ID NO:2, or SEQ ID NO:3; 59 of SEQ ID NO: 1, SEQ ID NO:2, or SEQ ID NO:3; 60 of SEQ ID NO: 1 or SEQ ID NO:2; 6 1 of SEQ ID NO: 1 or SEQ ID NO:2; 62 of SEQ ID NO: 1 or SEQ ID NO:2; 84 of SEQ ID NO: 1 or SEQ ID NO:2; 88 of SEQ ID NO: 1 or SEQ ID NO:2; 94 of SEQ ID NO: 1, SEQ ID NO:2, or SEQ ID NO:3; 96 of SEQ ID NO: 1, SEQ ID NO:2, or SEQ ID NO:3; 104 of SEQ ID NO: 1 or SEQ ID NO:2; 105 of SEQ ID NO: 1 or SEQ ID NO:2; 107 of SEQ ID NO: 1 or SEQ ID NO:2; 108 of SEQ ID NO: 1 or SEQ ID NO:2; 109 of SEQ ID NO: 1, SEQ ID NO:2, or SEQ ID NO:3; 110 of SEQ ID NO: 1 or SEQ ID NO:2; 111 of SEQ ID NO: 1 or SEQ ID NO:2; 112 of SEQ ID NO: 1, SEQ ID NO:2, or SEQ ID NO:3; 141 of SEQ ID NO: 1 or SEQ ID NO:2; 147 of SEQ ID NO: 1, SEQ ID NO:2, or SEQ ID NO:3; 154 of SEQ ID NO: 1 or SEQ ID NO:2; 179 of SEQ ID NO: 1, SEQ ID NO:2, or SEQ ID NO:3; 180 of SEQ ID NO: 1 or SEQ ID NO:2; 181 of SEQ ID NO: 1 or SEQ ID NO:2; 183 of SEQ ID NO: 1, SEQ ID NO:2, or SEQ ID NO:3; 184 of SEQ ID NO: 1, SEQ ID NO:2, or SEQ ID NO:3; 185 of SEQ ID NO: 1 or SEQ ID NO:2; 186 of SEQ ID NO: 1, SEQ ID NO:2, or SEQ ID NO:3; 187 of SEQ ID NO: 1 or SEQ ID NO:2; 188 of SEQ ID NO: 1 or SEQ ID NO:2; 189 of SEQ ID NO: 1 or SEQ ID NO:2; 198 of SEQ ID NO: 1 or SEQ ID NO:2; 204 of SEQ ID NO:3; 205 of SEQ ID NO: 1 or SEQ ID NO:2; 241 of SEQ ID NO:3; 242 of SEQ ID NO: 1 or SEQ ID NO:2; 247 of SEQ ID NO: 1 or SEQ ID NO:2; 247 of SEQ ID NO:3; 248 of SEQ ID NO: 1 or SEQ ID NO:2; 250 of SEQ ID NO:3; 25 1 of SEQ ID NO: 1 or SEQ ID NO:2; 264 of SEQ ID NO: 1, SEQ ID NO:2, or SEQ ID NO:3; 265 of SEQ ID NO: 1 or SEQ ID NO:2; and 286 of SEQ ID NO: 1 or SEQ ID NO:2, or the equivalent position in a Shiga toxin A Subunit polypeptide, conserved Shiga toxin effector polypeptide sub-region, and/or non-native, Shiga toxin effector polypeptide sequence (such as the Shiga toxin 1 A Subunit variant effector polypeptides shown in SEQ ID NOs: 4-6 or the Shiga-like toxin 2 A Subunit variant effector polypeptides shown in SEQ ID NOs: 7-18).

In some embodiments, the Shiga toxin effector polypeptides comprise, consist of, or consist essentially of a full-length or truncated Shiga toxin A Subunit with at least one substitution of an immunogenic residue and/or within an epitope region, wherein at least one amino acid substitution is to a non-conservative amino acid relative to a natively occurring amino acid positioned at one of the following native positions: 1 of SEQ ID NO: 1 or SEQ ID NO:2; 4 of SEQ ID NO: 1, SEQ ID NO:2, or SEQ ID NO:3; 8 of SEQ ID NO: 1, SEQ ID NO:2, or SEQ ID NO:3; 9 of SEQ ID NO: 1, SEQ ID NO:2, or SEQ ID NO:3; 11 of SEQ ID NO: 1, SEQ ID NO:2, or SEQ ID NO:3; 33 of SEQ ID NO: 1 or SEQ ID NO:2; 43 of SEQ ID NO: 1 or SEQ ID NO:2; 44 of SEQ ID NO: 1 or SEQ ID NO:2; 45 of SEQ ID NO: 1 or SEQ ID NO:2; 46 of SEQ ID NO: 1 or SEQ ID NO:2; 47 of SEQ ID NO: 1 or SEQ ID NO:2; 48 of SEQ ID NO: 1, SEQ ID NO:2, or SEQ ID NO:3; 49 of SEQ ID NO: 1 or SEQ ID NO:2; 50 of SEQ ID NO: 1 or SEQ ID NO:2; 51 of SEQ ID NO: 1 or SEQ ID NO:2; 53 of SEQ ID NO: 1 or SEQ ID NO:2; 54 of SEQ ID NO: 1 or SEQ ID NO:2; 55 of SEQ ID NO: 1 or SEQ ID NO:2; 56 of SEQ ID NO: 1 or SEQ ID NO:2; 57 of SEQ ID NO: 1 or SEQ ID NO:2; 58 of SEQ ID NO: 1, SEQ ID NO:2, or SEQ ID NO:3; 59 of SEQ ID NO: 1, SEQ ID NO:2, or SEQ ID NO:3; 60 of SEQ ID NO: 1 or SEQ ID NO:2; 6 1 of SEQ ID NO: 1 or SEQ ID NO:2; 62 of SEQ ID NO: 1 or SEQ ID NO:2; 84 of SEQ ID NO: 1 or SEQ ID NO:2; 88 of SEQ ID NO: 1 or SEQ ID NO:2; 94 of SEQ ID NO: 1, SEQ ID NO:2, or SEQ ID NO:3; 96 of SEQ ID NO: 1, SEQ ID NO:2, or SEQ ID NO:3; 104 of SEQ ID NO: 1 or SEQ ID NO:2; 105 of SEQ ID NO: 1 or SEQ ID NO:2; 107 of SEQ ID NO: 1 or SEQ ID NO:2; 108 of SEQ ID NO: 1 or SEQ ID NO:2; 109 of SEQ ID NO: 1, SEQ ID NO:2, or SEQ ID NO:3; 110 of SEQ ID NO: 1 or SEQ ID NO:2; 111 of SEQ ID NO: 1 or SEQ ID NO:2; 112 of SEQ ID NO: 1, SEQ ID NO:2, or SEQ ID NO:3; 141 of SEQ ID NO: 1 or SEQ ID NO:2; 147 of SEQ ID NO: 1, SEQ ID NO:2, or SEQ ID NO:3; 154 of SEQ ID NO: 1 or SEQ ID NO:2; 179 of SEQ ID NO: 1, SEQ ID NO:2, or SEQ ID NO:3; 180 of SEQ ID NO: 1 or SEQ ID NO:2; 181 of SEQ ID NO: 1 or SEQ ID NO:2; 183 of SEQ ID NO: 1, SEQ ID NO:2, or SEQ ID NO:3; 184 of SEQ ID NO: 1, SEQ ID NO:2, or SEQ ID NO:3; 185 of SEQ ID NO: 1 or SEQ ID NO:2; 186 of SEQ ID NO: 1, SEQ ID NO:2, or SEQ ID NO:3; 187 of SEQ ID NO: 1 or SEQ ID NO:2; 188 of SEQ ID NO: 1 or SEQ ID NO:2; 189 of SEQ ID NO: 1 or SEQ ID NO:2; 198 of SEQ ID NO: 1 or SEQ ID NO:2; 204 of SEQ ID NO:3; 205 of SEQ ID NO: 1 or SEQ ID NO:2; 241 of SEQ ID NO:3; 242 of SEQ ID NO: 1 or SEQ ID NO:2; 247 of SEQ ID NO: 1 or SEQ ID NO:2; 247 of SEQ ID NO:3; 248 of SEQ ID NO: 1 or SEQ ID NO:2; 250 of SEQ ID NO:3; 251 of SEQ ID NO: 1 or SEQ ID NO:2; 264 of SEQ ID NO: 1, SEQ ID NO:2, or SEQ ID NO:3; 265 of SEQ ID NO: 1 or SEQ ID NO:2; and 286 of SEQ ID NO: 1 or SEQ ID NO:2, or the equivalent position in a Shiga toxin A Subunit polypeptide, conserved Shiga toxin effector polypeptide sub-region, and/or non-native, Shiga toxin effector polypeptide sequence (such as the Shiga toxin effector polypeptide of any one of SEQ ID NOs: 4-18).

In some embodiments, the Shiga toxin effector polypeptides comprise, consist essentially of, or consist of a full-length or truncated Shiga toxin A Subunit with one or more mutations as compared to the native amino acid residue sequence which comprises at least one amino acid substitution of an immunogenic residue and/or within an epitope region, wherein at least one substitution occurs at the natively positioned amino acid position selected from the group consisting of: 1 of SEQ ID NO: 1 or SEQ ID NO:2; 11 of SEQ ID NO: 1 or SEQ ID NO:2; 45 of SEQ ID NO: 1 or SEQ ID NO:2; 54 of SEQ ID NO: 1, SEQ ID NO:2; 55 of SEQ ID NO: 1 or SEQ ID NO:2; 57 of SEQ ID NO: 1, SEQ ID NO:2; 59 of SEQ ID NO:1, SEQ ID NO:2; 60 of SEQ ID NO:1 or SEQ ID NO:2; 6 1 of SEQ ID NO:1 or SEQ ID NO:2; 110 of SEQ ID NO:1 or SEQ ID NO:2; 141 of SEQ ID NO: 1 or SEQ ID NO:2; 147 of SEQ ID NO: 1 or SEQ ID NO:2; 188 of SEQ ID NO: 1 or SEQ ID NO:2; 242 of SEQ ID NO: 1 or SEQ ID NO:2; 248 of SEQ ID NO: 1 or SEQ ID NO:2; and 25 1 of SEQ ID NO: 1 or SEQ ID NO:2.

In some embodiments, the Shiga toxin effector polypeptides comprise or consist essentially of a full-length or truncated Shiga toxin A Subunit with at least one amino acid substitution selected from the group consisting of: K I to A, G, V, L, I, F, M and H; T4 to A, G, V, L, I, F, M, and S; D6 to A, G, V, L, I, F, S, and Q; S8 to A, G, V, I, L, F, and M; T8 to A, G, V, I, L, F, M, and S; T9 to A, G, V, I, L, F, M, and S; S9 to A, G, V, L, I, F, and M; K I 1to A, G, V, L, I, F, M and H; T12 to A, G, V, I, L, F, M, and S; S33 to A, G, V, L, I, F, and M; S43 to A, G, V, L, I, F, and M; G44 to A and L; S45 to A, G, V, L, I, F, and M; T45 to A, G, V, L, I, F, and M; G46 to A and P; D47 to A, G, V, L, I, F, S, and Q; N48 to A, G, V, L, and M; L49 to A or G; F50; A5 1 to V; D53 to A, G, V, L, I, F, S, and Q; V54 to A, G, and L; R55 to A, G, V, L, I, F, M, Q, S, K, and H; G56 to A and P; 157 to A, G, M, and F; 57 to A, G, M, and F; D58 to A, G, V, F, I, F, S, and Q; P59 to A, G, and F; E60 to A, G, V, F, I, F, S, Q, N, D, M, and R; E61 to A, G, V, F, I, F, S, Q, N, D, M, and R; G62 to A; D94 to A, G, V, F, I, F, S, and Q; R84 to A, G, V, F, I, F, M, Q, S, K, and H; V88 to A and G; 188 to A, G, and V; D94; S96 to A, G, V, I, F, F, and M; T104 to A, G, V, I, F, F, M, and S; A105 to F; T107 to A, G, V, I, F, F, M, and S; S107 to A, G, V, F, I, F, and M; F108 to A, G, and M; S109 to A, G, V, I, F, F, and M; T109 to A, G, V, I, F, F, M, and S; G I 10 to A; D I 11 to A, G, V, F, I, F, S, and Q; SI 12 to A, G, V, F, I, F, and M; D141 to A, G, V, F, I, F, S, and Q; G147 to A; V154 to A and G; R179 to A, G, V, F, I, F, M, Q, S, K, and H; T180 to A, G, V, F, I, F, M, and S; T181 to A, G, V, F, I, F, M, and S; D183 to A, G, V, F, I, F, S, and Q; D184 to A, G, V, F, I, F, S, and Q; F185 to A, G, and V; S186 to A, G, V, I, F, F, and M; G187 to A; R188 to A, G, V, F, I, F, M, Q, S, K, and H; S189 to A, G, V, I, F, F, and M; D197 to A, G, V, F, I, F, S, and Q; D198 to A, G, V, F, I, F, S, and Q; R204 to A, G, V, F, I, F, M, Q, S, K, and H; R205 to A, G, V, F, I, F, M, Q, S, K and H; C242 to A, G, V, and S; S247 to A, G, V, I, F, F, and M; Y247 to A, G, V, F, I, F, and M; R247 to A, G, V, F, I, F, M, Q, S, K, and H; R248 to A, G, V, F, I, F, M, Q, S, K, and H; R250 to A, G, V, F, I, F, M, Q, S, K, and H; R25 1to A, G, V, F, I, F, M, Q, S, K, and H; C262 to A, G, V, and S; D264 to A, G, V, F, I, F, S, and Q; G264 to A; and T286 to A, G, V, F, I, F, M, and S.

In some embodiments, the Shiga toxin effector polypeptides comprise, consist of, or consist essentially of a full-length or truncated Shiga toxin A Subunit with at least one (such as at least two, three, four, five, six, seven, eight, nine, ten, eleven, twelve, thirteen or more) of the following amino acid substitutions: K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R247A, R248A, R250A, R251A, or D264A, G264A, T286A, and/or T286I. In some embodiments, the Shiga toxin effector polypeptides comprise, consist essentially of, or consist of a full-length or truncated Shiga toxin A Subunit with at least one (such as at least two, three, four, five, six, seven, eight, nine, ten, eleven, twelve, thirteen or more) of the following amino acid substitutions: K1A, S45I, V54I, R55L, I57F, P59F, E60T, E61L, G110A, D141A, G147A, R188A, C242S, R248A, and R251A. These epitope disrupting substitutions may be combined to form a de-immunized, Shiga toxin effector polypeptide with multiple substitutions per epitope region and/or multiple epitope regions disrupted while still retaining Shiga toxin effector function. For example, substitutions at the natively positioned K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R247A, R248A, R250A, R251A, or D264A, G264A, T286A, and/or T286I may be combined, where possible, with substitutions at the natively positioned residues K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R247A, R248A, R250A, R251A, or D264A, G264A, T286A, and/or T286I to create de-immunized, Shiga toxin effector polypeptides. For example, the Shiga toxin effector polypeptides may comprise, consist essentially of, or consist of a full-length or truncated Shiga toxin A Subunit comprising the following substitutions at native positions in a Shiga toxin A Subunit: K1A, S45I, V54I, R55L, I57F, P59F, E60T, E61L, G110A, G147A, C242S, R248A, and R251A. These substitutions correspond to those present in the Shiga toxin effector polypeptide of the exemplary cell-targeting molecule shown in any one of SEQ ID NOs: 24-27 and 97-100. For example, the Shiga toxin effector polypeptides may comprise, consist essentially of, or consist of a full-length or truncated Shiga toxin A Subunit comprising the following substitutions at native positions in a Shiga toxin A Subunit: S45I, V54I, R55L, I57F, P59F, E60T, E61F, G110A, R188A, C242S, R248A, and R251A. These substitutions correspond to those present in the Shiga toxin effector polypeptide of the exemplary cell-targeting molecule shown in any one of SEQ ID NOs: 28-29, 31-32, 34, 36, 101-102, 104-105, 106, and 108. For example, the Shiga toxin effector polypeptides may comprise, consist essentially of, or consist of a full-length or truncated Shiga toxin A Subunit comprising the following substitutions at native positions in a Shiga toxin A Subunit: S45I, V54I, R55L, I57F, P59F, E60T, E61L, G110A, D141A, R188A, C242S, R248A, and R251A. These substitutions correspond to those present in the Shiga toxin effector polypeptide of the exemplary cell-targeting molecule shown in any one of SEQ ID NOs: 30 or 103.

In some embodiments, the Shiga toxin effector polypeptide comprises (1) a Shiga toxin A1 fragment derived region having a carboxy-terminus and (2) a disrupted furin-cleavage motif at the carboxy-terminus of the Shiga toxin A1 fragment region. Improving the stability of connections between the Shiga toxin component and other components of cell-targeting molecules, e.g., cell-targeting binding regions, can improve their toxicity profiles after administration to organisms by reducing non-specific toxicities caused by the breakdown of the connection and loss of cell-targeting, such as, e.g., as a result of proteolysis. In some embodiments, the protease-cleavage resistant Shiga toxin effector polypeptide has a carboxy-terminal truncation as compared to the carboxy-terminus of a wild-type Shiga toxin A Subunit.

Shiga toxin A Subunits of members of the Shiga toxin family comprise a conserved, furin-cleavage site at the carboxy-terminal of their A1 fragment regions important for Shiga toxin function. Furin-cleavage site motifs and furin-cleavage sites can be identified by the skilled worker using standard techniques and/or by using the information herein.

In some embodiments, the Shiga toxin effector polypeptide comprising a disrupted furin-cleavage motif is directly fused by a peptide bond to a molecular moiety comprising an amino acid, peptide, and/or polypeptide wherein the fused structure involves a single, continuous polypeptide. In these fusion embodiments, the amino acid sequence following the disrupted furin-cleavage motif may be designed not to create a de novo, furin-cleavage site at the fusion junction and the molecular moiety may be chosen so as not to comprise any furin cleavage sites.

In some embodiments, the Shiga toxin effector polypeptide comprises an embedded or inserted epitope-peptide and a Shiga toxin A1 fragment derived region. In some embodiments, the epitope-peptide is a heterologous, T-cell epitope-peptide, such as, e.g., an epitope considered heterologous to Shiga toxin A Subunits. In some embodiments, the Shiga toxin effector polypeptide comprises an embedded or inserted epitope-peptide within the Shiga toxin A1 fragment region. In some embodiments, the epitope-peptide is a CD8+ T-cell epitope. In some embodiments, the CD8+ T-cell epitope-peptide has a binding affinity to a MHC class I molecule characterized by a dissociation constant (KD) of 10⁴ molar or less and/or the resulting MHC class 1-epitope-peptide complex has a binding affinity to a T-cell receptor (TCR) characterized by a dissociation constant (KD) of 104 molar or less.

Any of the de-immunized, Shiga toxin effector polypeptide sub-regions and/or epitope disrupting mutations; the protease-cleavage resistant, Shiga toxin effector polypeptide sub-regions and/or disrupted furin-cleavage motifs; or the protease-cleavage resistant, Shiga toxin effector polypeptide sub-regions and/or disrupted furin-cleavage motifs described herein may be used alone or in combination with each individual embodiment described herein, including methods described herein.

### HER2 Binding Molecules

Provided herein are various HER2 binding molecules, each comprising (1) a HER2 binding region, and (2) a Shiga toxin effector polypeptide. In some embodiments, a HER2 binding molecule comprises a binding region capable of specifically binding an extracellular part of HER2, and a Shiga toxin effector polypeptide capable of exhibiting one or more Shiga toxin A subunit effector functions, such as, cytostasis, cytotoxicity, catalytic activity, promoting cellular internalization, directing intracellular routing to a certain subcellular compartment(s), and intracellular delivery of a material(s). The association of a HER2 binding region with a Shiga toxin effector polypeptide allows for the engineering of therapeutic and diagnostic molecules with desirable characteristics, such as de-immunization, potent cytotoxicity, efficient intracellular routing, T-cell hyper-immunization, molecular stability, and in vivo tolerability at high dosages as compared to certain reference molecules.

In some embodiments, the binding molecules comprise a Shiga toxin A subunit effector polypeptide and a binding region capable of binding specifically to a HER2 extracellular domain. In some embodiments, the binding region comprises a heavy chain variable domain (VH) comprising a HCDR1, a HCDR2, and a HCDR3. In some embodiments, the binding region comprises a light chain variable domain (VL) comprising a LCDR1, a LCDR2, and a LCDR3. In some embodiments, the binding region comprises a VH and a VL.

One non-limiting example of a cell-targeting molecule is a Shiga toxin effector polypeptide fused to a proteinaceous, cell-targeting, binding region, such as, e.g., an immunoglobulin or immunoglobulin-type binding region. For example, the cell-targeting molecules may comprise an immunoglobulin binding region capable of specifically binding an extracellular part of HER2, and comprising a polypeptide comprising one or more of: an antibody variable fragment, a single-domain antibody fragment, a single-chain variable fragment, a Fd fragment, an antigen-binding fragment, an autonomous VH domain, a VHH fragment derived from a camelid antibody, a heavy-chain antibody domain derived from a cartilaginous fish antibody, a VNAR fragment, and an immunoglobulin new antigen receptor.

In some embodiments, a HER2 binding molecule comprises a cytotoxic Shiga toxin A subunit effector polypeptide; and a binding region capable of specifically binding an extracellular part of human HER2, wherein the binding region comprises: an immunoglobulin heavy chain variable region comprising: a CDR1 comprising the sequence of SEQ ID NO: 57; a CDR2 comprising the sequence of SEQ ID NO: 58; and a CDR3 comprising the sequence of SEQ ID NO: 59; and an immunoglobulin light chain variable region comprising: a CDR1 comprising the sequence of SEQ ID NO: 60; a CDR2 comprising the sequence of SEQ ID NO: 61; and a CDR3 comprising the sequence of SEQ ID NO: 62. In some embodiments, the Shiga toxin A subunit effector polypeptide and binding region are fused, forming a continuous polypeptide.

Sequences of exemplary HER2 binding molecules are provided below in Table 6. In some embodiments, a HER2 binding molecule comprises the sequence of any one of SEQ ID NO: 22-36 or 97-108. In some embodiments, the HER2 binding molecule comprises a sequence that is at least 90%, at least 91%, at least 92%, at least 93%, at least 94%, at least 95%, at least 96% at least 97%, at least 98%, or at least 99% identical to any one of SEQ ID NO: 22-36 or 97-108. In some embodiments, the HER2 binding molecule comprises a sequence that has 1, 2, 3, 4, 5, 6, 7, 8, 9, 10, 11, 12, 13, 14, 15, 16, 17, 18, 19, 20, 21, 22, 23, 24, or 25, or more, amino acid substitutions relative to any one of SEQ ID NO: 22-36 or 97-108. In some embodiments, the HER2 binding molecule comprises the sequence of SEQ ID NO: 29, or a sequence that is at least at least 95%, at least 96%, at least 97%, at least 98%, or at least 99% identical thereto.

TABLE 6
Exemplary HER2 binding molecules

| | SEQ ID | |
|---|---|---|
| Description | NO | Sequence |
| 114773 | 22 | MKEFTLDFSTAKTYVDSLNVIRSAIGTPLQTISSGGTSLLMIDSGSG |
| | | DNLFAVDVRGIDPEEGRFNNLRLIVERNNLYVTGFVNRTNNVFYRFA |
| | | DFSHVTFPGTTAVTLSGDSSYTTLQRVAGISRIGMQINRHSLTTSYL |
| | | DLMSHSGTSLTQSVARAMLRFVTVTAEALRFRQIQRGFRTTLDDLSG |
| | | RSYVMTAEDVDLTLNWGRLSSVLPDYHGQDSVRVGRISFGSINAILG |
| | | SVALILNCHHHASAVAAEFPKPSTPPGSSGGAPDIQMTQSPSSLSAS |
| | | VGDRVTITCRASQDVNTAVAWYQQKPGKAPKLLIYSASFLYSGVPSR |
| | | FSGSRSGTDFTLTISSLQPEDFATYYCQQHYTTPPTFGQGTKVEIKG |
| | | GGGSEVQLVESGGGLVQPGGSLRLSCAASGFNIKDTYIHWVRQAPGK |
| | | GLEWVARIYPTNGYTRYADSVKGRFTISADTSKNTAYLQMNSLRAED |
| | | TAVYYCSRWGGDGFYAMDYWGQGTLVTVSSA |
| 115172 | 23 | MKEFTLDFSTAKTYVDSLNVIRSAIGTPLQTISSGGTSLLMIDSGSG |
| | | DNLFAVDVRGIDPEEGRFNNLRLIVERNNLYVTGFVNRTNNVFYRFA |
| | | DFSHVTFPGTTAVTLSGDSSYTTLQRVAGISRTGMQINRHSLTTSYL |
| | | DLMSHSGTSLTQSVARAMLRFVTVTAEALRFRQIQRGFRTTLDDLSG |
| | | RSYVMTAEDVDLTLNWGRLSSVLPDYHGQDSVRVGRISFGSINAILG |
| | | SVALILNCHHHASAVAAEFPKPSTPPGSSGGAPQVQLQQSGPELKKP |
| | | GETVKISCKASGYPFTNYGMNWVKQAPGQGLKWMGWINTSTGESTFA |
| | | DDFKGRFDFSLETSANTAYLQINNLKSEDSATYFCARWEVYHGYVPY |
| | | WGQGTTVTVSSGGGGSGGGGSGGGGSGGGGSGGGGSDIQMTQSPSSL |
| | | SASVGDRVTITCKASQDVYNAVAWYQQKPGQSPKLLIYSASSRYTGV |
| | | PSRFTGSGSGPDFTFTISSVQAEDLAVYFCQQHFRTPFTFGSGTKLE |
| | | IK |
| 114778 | 24 | MAEFTLDFSTAKTYVDSLNVIRSAIGTPLQTISSGGTSLLMIDSGIG |
| | | DNLFAVDILGFDFTLGRFNNLRLIVERNNLYVTGFVNRTNNVFYRFA |
| | | DFSHVTFPGTTAVTLSADSSYTTLQRVAGISRIGMQINRHSLTTSYL |
| | | DLMSHSATSLTQSVARAMLRFVTVTAEALRFRQIQRGFRTTLDDLSG |
| | | RSYVMTAEDVDLTLNWGRLSSVLPDYHGQDSVRVGRISFGSINAILG |
| | | SVALILNSHHHASAVAAEFPKPSTPPGSSGGAPDIQMTQSPSSLSAS |
| | | VGDRVTITCRASQDVNTAVAWYQQKPGKAPKLLIYSASFLYSGVPSR |
| | | FSGSRSGTDFTLTISSLQPEDFATYYCQQHYTTPPTFGQGTKVEIKG |
| | | GGGSEVQLVESGGGLVQPGGSLRLSCAASGFNIKDTYIHWVRQAPGK |
| | | GLEWVARIYPTNGYTRYADSVKGRFTISADTSKNTAYLQMNSLRAED |
| | | TAVYYCSRWGGDGFYAMDYWGQGTLVTVSSA |
| 114795 | 25 | MAEFTLDFSTAKTYVDSLNVIRSAIGTPLQTISSGGTSLLMIDSGIG |
| | | DNLFAVDILGFDFTLGRFNNLRLIVERNNLYVTGFVNRINNVFYRFA |
| | | DFSHVTFPGTTAVTLSADSSYTTLQRVAGISRTGMQINRHSLTTSYL |
| | | DLMSHSATSLTQSVARAMLRFVTVTAEALRFRQIQRGFRTTLDDLSG |
| | | RSYVMTAEDVDLTLNWGRLSSVLPDYHGQDSVRVGRISFGSINAILG |
| | | SVALILNSHHHASAVAAEFPKPSTPPGSSGGAPQVQLLQSGAELKKP |
| | | GESLKISCKGSGYSFTSYWIAWVRQMPGKGLEYMGLIYPGDSDTKYS |
| | | PSFQGQVTISVDKSVSTAYLQWSSLKPSDSAVYFCARHDVGYCSSSN |
| | | CAKWPEYFQHWGQGTLVTVSSGGGGSQSVLTQPPSVSAAPGQKVTIS |
| | | CSGSSSNIGNNYVSWYQQLPGTAPKLLIYGHTNRPAGVPDRFSGSKS |
| | | GTSASLAISGFRSEDEADYYCAAWDDSLSGWVFGGGTKLTVLA |
| 114791 | 26 | MAEFTLDFSTAKTYVDSLNVIRSAIGTPLQTISSGGTSLLMIDSGIG |
| | | DNLFAVDILGFDFTLGRFNNLRLIVERNNLYVTGFVNRTNNVFYRFA |
| | | DFSHVTFPGTTAVTLSADSSYTTLQRVAGISRTGMQINRHSLTTSYL |
| | | DLMSHSATSLTQSVARAMLRFVTVTAEALRFRQIQRGFRTTLDDLSG |
| | | RSYVMTAEDVDLTLNWGRLSSVLPDYHGQDSVRVGRISFGSINAILG |
| | | SVALILNSHHHASAVAAEFPKPSTPPGSSGGAPQVQLQQSGPELKKP |
| | | GETVKISCKASGYPFTNYGMNWVKQAPGQGLKWMGWINTSTGESTFA |
| | | DDFKGRFDFSLETSANTAYLQINNLKSEDSATYFCARWEVYHGYVPY |
| | | WGQGTTVTVSSGGGGSDIQLTQSHKFLSTSVGDRVSITCKASQDVYN |
| | | AVAWYQQKPGQSPKLLIYSASSRYTGVPSRFTGSGSGPDFTFTISSV |
| | | QAEDLAVYFCQQHFRTPFTFGSGTKLEIK |
| SLTA-DI- | 27 | MAEFTLDFSTARTYVDSLNVIRSAIGTPLQTISSGGTSLLMIDSGIG |
| 1:scFv4 | | DNLFAVDILGFDFTLGRFNNLRLIVERNNLYVTGFVNRTNNVFYRFA |
| | | DFSHVTFPGTTAVTLSADSSYTTLQRVAGISRTGMQINRHSITTSYL |
| | | DLMSHSATSLTQSVARAMLRFVTVTAEALRFRQIQRGFRTTLDDLSG |
| | | RSYVMTAEDVDLTLNWGRLSSVLPDYHGQDSVRVGRISFGSINAILG |
| | | SVALILNSHHHASAVAAEFPKPSTPPGSSGGAPDIQMTQSPSSLSAS |
| | | VGDRVTITCKASQDVSIGVAWYQQKPGKAPKLLIYSASYRYTGVPSR |
| | | FSGSGSGTDFTLTISSLQPEDFATYYCQQYYTYPYTFGQGTKVEIKG |
| | | GGGSEVQLVESGGGLVQPGGSLRLSCAASGFTFTDYTMDWVRQAPGK |
| | | GLEWVADVNPNSGGSIYNQRFKGRFTLSVDRSKNTLYLQMNSLRAED |
| | | TAVYYCARNLGPSFYFDYWGQGTLVTVSSA |
| 114912 | 28 | MKEFTLDFSTAKTYVDSLNVIRSAIGTPLQTISSGGTSLLMIDSGIG |
| | | DNLFAVDILGFDFTLGRFNNLRLIVERNNLYVTGFVNRTNNVFYRFA |
| | | DFSHVTFPGTTAVTLSADSSYTTLQRVAGISRTGMQINRHSLTTSYL |
| | | DLMSHSGTSLTQSVARAMLRFVTVTAEALRFRQIQRGFRTTLDDLSG |
| | | ASYVMTAEDVDLTLNWGRLSSVLPDYHGQDSVRVGRISFGSINAILG |
| | | SVALILNSHHHASAVAAEFPKPSTPPGSSGGAPDIQMTQSPSSLSAS |
| | | VGDRVTITCRASQDVNTAVAWYQQKPGKAPKLLIYSASFLYSGVPSR |
| | | FSGSRSGTDFTLTISSLQPEDFATYYCQQHYTTPPTFGQGTKVEIKG |
| | | GGGSGGGGSGGGGSGGGGSGGGGSEVQLVESGGGLVQPGGSLRLSCA |
| | | ASGFNIKDTYIHWVRQAPGKGLEWVARIYPTNGYTRYADSVKGRFTI |
| | | SADTSKNTAYLQMNSLRAEDTAVYYCSRWGGDGFYAMDYWGQGTLVT |
| | | VSS |
| 115111 | 29 | MKEFTLDFSTAKTYVDSLNVIRSAIGTPLQTISSGGTSLLMIDSGIG |
| | | DNLFAVDILGFDFTLGRFNNLRLIVERNNLYVTGFVNRTNNVFYRFA |
| | | DFSHVTFPGTTAVTLSADSSYTTLQRVAGISRTGMQINRHSLTTSYL |
| | | DLMSHSGTSLTQSVARAMLRFVTVTAEALRFRQIQRGFRTTLDDLSG |
| | | ASYVMTAEDVDLTLNWGRLSSVLPDYHGQDSVRVGRISFGSINAILG |
| | | SVALILNSHHHASAVAAEFPKPSTPPGSSGGAPQVQLQQSGPELKKP |
| | | GETVKISCKASGYPFTNYGMNWVKQAPGQGLKWMGWINTSTGESTFA |
| | | DDFKGRFDFSLETSANTAYLQINNLKSEDSATYFCARWEVYHGYVPY |
| | | WGQGTTVTVSSGGGGSGGGGSGGGGSGGGGSGGGGSDIQMTQSPSSL |
| | | SASVGDRVTITCKASQDVYNAVAWYQQKPGQSPKLLIYSASSRYTGV |
| | | PSRFTGSGSGPDFTFTISSVQAEDLAVYFCQQHFRTPFTFGSGTKLE |
| | | IK |
| 115411 | 30 | MREFTLDFSTARTYVDSLNVIRSAIGTPLQTISSGGTSLLMIDSGIG |
| | | DNLFAVDILGFDFTLGRFNNLRLIVERNNLYVTGFVNRTNNVFYRFA |
| | | DFSHVTFPGTTAVTLSADSSYTTLQRVAGISRIGMQINRHSLTTSYL |
| | | ALMSHSGTSLTQSVARAMLRFVTVTAEALRFRQIQRGFRTTLDDLSG |
| | | ASYVMTAEDVDLTLNWGRLSSVLPDYHGQDSVRVGRISFGSINAILG |
| | | SVALILNSHHHASAVAASPSTPPTPSPSTPPASQVQLVQSGPEVKKP |
| | | GASVKVSCKASGYPFTNYGMNWVRQAPGQGLEWMGWINTSTGESTFA |
| | | DDFKGRVTMTTDTSTSTTYMELRSLRPDDTAVYFCARWEVYHGYVPY |
| | | WGQGTLVTVSSGGGGSGGGGSGGGGSGGGGSGGGGSDIQMTQSPSSL |
| | | SASIGDRVTITCKASQDVYNAVAWYQQKPGEAPKLLVYSASSRYTGV |
| | | PSRFSGSGSGTDFTFTISSLQPEDIATYFCQQHFRTPFTFAPGTKLE |
| | | IK |
| 114898 | 31 | MKEFTLDFSTAKTYVDSLNVIRSAIGTPLQTISSGGTSLLMIDSGIG |
| | | DNLFAVDILGFDFTLGRFNNLRLIVERNNLYVTGFVNRTNNVFYRFA |
| | | DFSHVTFPGTTAVTLSADSSYTTLQRVAGISRTGMQINRHSLTTSYL |
| | | DLMSHSGTSLTQSVARAMLRFVTVTAEALRFRQIQRGFRTTLDDLSG |
| | | ASYVMTAEDVDLTLNWGRLSSVLPDYHGQDSVRVGRISFGSINAILG |
| | | SVALILNSHHHASAVAAAHHSEDPSSKAPKAPEVQLVESGGGLVQAG |
| | | GSLRLSCAASGITFSINTMGWYRQAPGKQRELVALISSIGDTYYADS |
| | | VKGRFTISRDNAKNTVYLQMNSLKPEDTAVYYCKRFRTAAQGTDYWG |
| | | QGTQVTVSSA |
| 115195 | 32 | MKEFTLDFSTAKTYVDSLNVIRSAIGTPLQTISSGGTSLLMIDSGIG |
| | | DNLFAVDILGFDFTLGRFNNLRLIVERNNLYVTGFVNRTNNVFYRFA |
| | | DFSHVTFPGTTAVTLSADSSYTTLQRVAGISRTGMQINRHSLTTSYL |
| | | DLMSHSGTSLTQSVARAMLRFVTVTAEALRFRQIQRGFRTTLDDLSG |
| | | ASYVMTAEDVDLTLNWGRLSSVLPDYHGQDSVRVGRISFGSINAILG |
| | | SVALILNSHHHASAVAAEFPKPSTPPGSSGGAPQVQLQQSGPELKKP |
| | | GETVKISCKASGYPFTNYGMNWVKQAPGQGLKWMGWINTSTGESTFA |
| | | DDFKGRFDFSLETSANTAYLQINNLKSEDSATYFCARWEVYHGYVPY |
| | | WGQGTTVTVSSGGGGSDIQMTQSPSSLSASVGDRVTITCKASQDVYN |
| | | AVAWYQQKPGQSPKLLIYSASSRYTGVPSRFTGSGSGPDFTFTISSV |
| | | QAEDLAVYFCQQHFRTPFTFGSGTKLEIK |
| 115194 | 33 | MKEFTLDFSTAKTYVDSLNVIRSAIGTPLQTISSGGTSLLMIDSGSG |
| | | DFSHVTFPGTTAVTLSGDSSYTTLQRVAGISRTGMQINRHSLTTSYL |
| | | DNLFAVDVRGIDPEEGRFNNLRLIVERNNLYVTGFVNRINNVFYRFA |
| | | DLMSHSGTSLTQSVARAMLRFVTVTAEALRFRQIQRGFRTTLDDLSG |
| | | RSYVMTAEDVDLTLNWGRLSSVLPDYHGQDSVRVGRISFGSINAILG |
| | | SVALILNCHHHASAVAAEFPKPSTPPGSSGGAPQVQLQQSGPELKKP |
| | | GETVKISCKASGYPFTNYGMNWVKQAPGQGLKWMGWINTSTGESTFA |
| | | DDFKGRFDFSLETSANTAYLQINNLKSEDSATYFCARWEVYHGYVPY |
| | | WGQGTTVTVSSGGGGSDIQMTQSPSSLSASVGDRVTITCKASQDVYN |
| | | AVAWYQQKPGQSPKLLIYSASSRYTGVPSRFTGSGSGPDFTFTISSV |
| | | QAEDLAVYFCQQHFRTPFTFGSGTKLEIK |
| 115645 | 34 | MKEFTLDFSTAKTYVDSLNVIRSAIGTPLQTISSGGTSLLMIDSGIG |
| | | DNLFAVDILGFDFTLGRFNNLRLIVERNNLYVTGFVNRTNNVFYRFA |
| | | DFSHVTFPGTTAVTLSADSSYTTLQRVAGISRIGMQINRHSLTTSYL |
| | | DLMSHSGTSLTQSVARAMLRFVTVTAEALRFRQIQRGFRTTLDDLSG |
| | | ASYVMTAEDVDLTLNWGRLSSVLPDYHGQDSVRVGRISFGSINAILG |
| | | SVALILNSHHHASAVAAEVQLVESGGGLVQAGGSLRLSCAASGITFS |
| | | INTMGWYRQAPGKQRELVALISSIGDTYYADSVKGRFTISRDNAKNT |
| | | VYLQMNSLKPEDTAVYYCKRFRTAAQGTDYWGQGTQVTVSSA |
| 115845 | 35 | MKEFTLDFSTAKTYVDSLNVIRSAIGTPLQTISSGGTSLLMIDSGIG |
| | | DNLFAVDILGFDFTLGRFNNLRLIVERNNLYVTGFVNRTNNVFYRFA |
| | | DFSHVTFPGTTAVTLSADSSYTTLQRVAGISRTGMQINRHSLTTSYI |
| | | ALMSHSGTSLTQSVARAMLRFVTVTAEALRFRQIQRGFRTTLDDLSG |
| | | ASYVMTAEDVDLTLNWGRLSSVLPDYHGQDSVRVGRISFGSINAILG |
| | | SVALILNSHHHASAVAAQVQLQESGGGSVQAGGSLKLICAASGYIFN |
| | | SCGMGWYRQSPGRERELVSRISGDGDTWHKESVKGRFTISQDNVKKT |
| | | LYLQMNSLKPEDTAVYFCAVCYNLETYWGQGTQVTVSSHHHHHH |
| SLTA-DI- | 36 | MKEFTLDFSTAKTYVDSLNVIRSAIGTPLQTISSGGTSLLMIDSGIG |
| 2::scFv8 | | DNLFAVDILGFDFTLGRFNNLRLIVERNNLYVTGFVNRINNVFYRFA |
| | | DFSHVTFPGTTAVTLSADSSYTTLQRVAGISRTGMQINRHSLITSYL |
| | | DLMSHSGTSLTQSVARAMLRFVTVTAEALRFRQIQRGFRTTLDDLSG |
| | | ASYVMTAEDVDLTLNWGRLSSVLPDYHGQDSVRVGRISFGSINAILG |
| | | SVALILNSHHHASAVAAEFPKPSTPPGSSGGAPQVQLQQSGPELKKP |
| | | GETVKISCKASGYPFTNYGMNWVKQAPGQGLKWMGWINTSTGESTFA |
| | | DDFKGRFDFSLETSANTAYLQINNLKSEDSATYFCARWEVYHGYVPY |
| | | WGQGTTVTVSSGGGGSGGGGSGGGGSGGGGSDIQMTQSPSSLSASVG |
| | | DRVTITCKASQDVYNAVAWYQQKPGQSPKLLIYSASSRYTGVPSRFT |
| | | GSGSGPDFTFTISSVQAEDLAVYFCQQHFRTPFTFGSGTKLEIK |
| HER2- | 97 | AEFTLDFSTAKTYVDSLNVIRSAIGTPLQTISSGGTSLLMIDSGIGD |
| targeting | | NLFAVDILGFDFTLGRFNNLRLIVERNNLYVTGFVNRTNNVFYRFAD |
| molecule #1 | | FSHVTFPGTTAVTLSADSSYTTLQRVAGISRTGMQINRHSLITSYLD |
| | | LMSHSATSLTQSVARAMLRFVTVTAEALRFRQIQRGFRTTLDDLSGR |
| | | SYVMTAEDVDLTLNWGRLSSVLPDYHGQDSVRVGRISFGSINAILGS |
| | | VALILNSHHHASAVAAEFPKPSTPPGSSGGAPDIQMTQSPSSLSASV |
| | | GDRVTITCRASQDVNTAVAWYQQKPGKAPKLLIYSASFLYSGVPSRF |
| | | SGSRSGTDFTLTISSLQPEDFATYYCQQHYTTPPTFGQGTKVEIKGG |
| | | GGSEVQLVESGGGLVQPGGSLRLSCAASGFNIKDTYIHWVRQAPGKG |
| | | LEWVARIYPTNGYTRYADSVKGRFTISADTSKNTAYLQMNSLRAEDT |
| | | AVYYCSRWGGDGFYAMDYWGQGTLVTVSSA |
| HER2- | 98 | AEFTLDFSTAKTYVDSLNVIRSAIGTPLQTISSGGTSLLMIDSGIGD |
| targeting | | NLFAVDILGFDFTLGRFNNLRLIVERNNLYVTGFVNRTNNVFYRFAD |
| molecule #2 | | FSHVTFPGTTAVTLSADSSYTTLQRVAGISRTGMQINRHSLTTSYLD |
| | | LMSHSATSLTQSVARAMLRFVTVTAEALRFRQIQRGFRTTLDDLSGR |
| | | SYVMTAEDVDLTLNWGRLSSVLPDYHGQDSVRVGRISFGSINAILGS |
| | | VALILNSHHHASAVAAEFPKPSTPPGSSGGAPQVQLLQSGAELKKPG |
| | | ESLKISCKGSGYSFTSYWIAWVRQMPGKGLEYMGLIYPGDSDTKYSP |
| | | SFQGQVTISVDKSVSTAYLQWSSLKPSDSAVYFCARHDVGYCSSSNC |
| | | AKWPEYFQHWGQGTLVTVSSGGGGSQSVLTQPPSVSAAPGQKVTISC |
| | | SGSSSNIGNNYVSWYQQLPGTAPKLLIYGHTNRPAGVPDRFSGSKSG |
| | | TSASLAISGFRSEDEADYYCAAWDDSLSGWVFGGGTKLTVLA |
| HER2- | 99 | AEFTLDFSTAKTYVDSLNVIRSAIGTPLQTISSGGTSLLMIDSGIGD |
| targeting | | NLFAVDILGFDFTLGRFNNLRLIVERNNLYVTGFVNRTNNVFYRFAD |
| molecule #3 | | FSHVTFPGTTAVTLSADSSYTTLQRVAGISRTGMQINRHSLTTSYLD |
| | | LMSHSATSLTQSVARAMLRFVTVTAEALRFRQIQRGFRTTLDDLSGR |
| | | SYVMTAEDVDLTLNWGRLSSVLPDYHGQDSVRVGRISFGSINAILGS |
| | | VALILNSHHHASAVAAEFPKPSTPPGSSGGAPQVQLQQSGPELKKPG |
| | | ETVKISCKASGYPFTNYGMNWVKQAPGQGLKWMGWINTSTGESTFAD |
| | | DFKGRFDFSLETSANTAYLQINNLKSEDSATYFCARWEVYHGYVPYW |
| | | GQGTTVTVSSGGGGSDIQLTQSHKFLSTSVGDRVSITCKASQDVYNA |
| | | VAWYQQKPGQSPKLLIYSASSRYTGVPSRFTGSGSGPDFTFTISSVQ |
| | | AEDLAVYFCQQHFRTPFTFGSGTKLEIK |
| HER2- | 100 | AEFTLDFSTAKTYVDSLNVIRSAIGTPLQTISSGGTSLLMIDSGIGD |
| targeting | | NLFAVDILGFDFTLGRFNNLRLIVERNNLYVTGFVNRTNNVFYRFAD |
| molecule #4 | | FSHVTFPGTTAVTLSADSSYTTLQRVAGISRTGMQINRHSLTTSYLD |
| | | LMSHSATSLTQSVARAMLRFVTVTAEALRFRQIQRGFRTTLDDLSGR |
| | | SYVMTAEDVDLTLNWGRLSSVLPDYHGQDSVRVGRISFGSINAILGS |
| | | VALILNSHHHASAVAAEFPKPSTPPGSSGGAPDIQMTQSPSSLSASV |
| | | GDRVTITCKASQDVSIGVAWYQQKPGKAPKLLIYSASYRYTGVPSRF |
| | | SGSGSGTDFTLTISSLQPEDFATYYCQQYYTYPYTFGQGTKVEIKGG |
| | | GGSEVQLVESGGGLVQPGGSLRLSCAASGFTFTDYTMDWVRQAPGKG |
| | | LEWVADVNPNSGGSIYNQRFKGRFTLSVDRSKNTLYLQMNSLRAEDT |
| | | AVYYCARNLGPSFYFDYWGQGTLVTVSSA |
| HER2- | 101 | KEFTLDFSTAKTYVDSLNVIRSAIGTPLQTISSGGTSLLMIDSGIGD |
| targeting | | NLFAVDILGFDFTLGRFNNLRLIVERNNLYVTGFVNRINNVFYRFAD |
| molecule #5 | | FSHVTFPGTTAVTLSADSSYTTLQRVAGISRTGMQINRHSLTTSYLD |
| | | LMSHSGTSLTQSVARAMLRFVTVTAEALRFRQIQRGFRTTLDDLSGA |
| | | SYVMTAEDVDLTLNWGRLSSVLPDYHGQDSVRVGRISFGSINAILGS |
| | | VALILNSHHHASAVAAEFPKPSTPPGSSGGAPDIQMTQSPSSLSASV |
| | | GDRVTITCRASQDVNTAVAWYQQKPGKAPKLLIYSASFLYSGVPSRF |
| | | SGSRSGTDFTLTISSLQPEDFATYYCQQHYTTPPTFGQGTKVEIKGG |
| | | GGSGGGGSGGGGSGGGGSGGGGSEVQLVESGGGLVQPGGSLRLSCAA |
| | | SGFNIKDTYIHWVRQAPGKGLEWVARIYPTNGYTRYADSVKGRFTIS |
| | | ADTSKNTAYLQMNSLRAEDTAVYYCSRWGGDGFYAMDYWGQGTLVTV |
| | | SS |
| HER2- | 102 | KEFTLDFSTAKTYVDSLNVIRSAIGTPLQTISSGGTSLLMIDSGIGD |
| targeting | | NLFAVDILGFDFTLGRFNNLRLIVERNNLYVTGFVNRINNVFYRFAD |
| molecule #6 | | FSHVTFPGTTAVTLSADSSYTTLQRVAGISRTGMQINRHSLTTSYLD |
| | | LMSHSGTSLTQSVARAMLRFVTVTAEALRFRQIQRGFRTTLDDLSGA |
| | | SYVMTAEDVDLTLNWGRLSSVLPDYHGQDSVRVGRISFGSINAILGS |
| | | VALILNSHHHASAVAAEFPKPSTPPGSSGGAPQVQLQQSGPELKKPG |
| | | ETVKISCKASGYPFTNYGMNWVKQAPGQGLKWMGWINTSTGESTFAD |
| | | DFKGRFDFSLETSANTAYLQINNLKSEDSATYFCARWEVYHGYVPYW |
| | | GQGTTVTVSSGGGGSGGGGSGGGGSGGGGSGGGGSDIQMTQSPSSLS |
| | | ASVGDRVTITCKASQDVYNAVAWYQQKPGQSPKLLIYSASSRYTGVP |
| | | SRFTGSGSGPDFTFTISSVQAEDLAVYFCQQHFRTPFTFGSGTKLEI |
| | | K |
| HER2- | 103 | REFTLDFSTARTYVDSLNVIRSAIGTPLQTISSGGTSLLMIDSGIGD |
| targeting | | NLFAVDILGFDFTLGRFNNLRLIVERNNLYVTGFVNRINNVFYRFAD |
| molecule #7 | | FSHVTFPGTTAVTLSADSSYTTLQRVAGISRTGMQINRHSLTTSYLA |
| | | LMSHSGTSLTQSVARAMLRFVTVTAEALRFRQIQRGFRTTLDDLSGA |
| | | SYVMTAEDVDLTLNWGRLSSVLPDYHGQDSVRVGRISFGSINAILGS |
| | | VALILNSHHHASAVAASPSTPPTPSPSTPPASQVQLVQSGPEVKKPG |
| | | ASVKVSCKASGYPFTNYGMNWVRQAPGQGLEWMGWINTSTGESTFAD |
| | | DFKGRVTMTTDTSTSTTYMELRSLRPDDTAVYFCARWEVYHGYVPYW |
| | | GQGTLVTVSSGGGGSGGGGSGGGGSGGGGSGGGGSDIQMTQSPSSLS |
| | | ASIGDRVTITCKASQDVYNAVAWYQQKPGEAPKLLVYSASSRYTGVP |
| | | SRFSGSGSGTDFTFTISSLQPEDIATYFCQQHFRTPFTFAPGTKLEI |
| | | K |
| HER2- | 104 | KEFTLDFSTAKTYVDSLNVIRSAIGTPLQTISSGGTSLLMIDSGIGD |
| targeting | | NLFAVDILGFDFTLGRFNNLRLIVERNNLYVTGFVNRTNNVFYRFAD |
| molecule #8 | | FSHVTFPGTTAVTLSADSSYTTLQRVAGISRTGMQINRHSLTTSYLD |
| | | LMSHSGTSLTQSVARAMLRFVTVTAEALRFRQIQRGFRTTLDDLSGA |
| | | SYVMTAEDVDLTLNWGRLSSVLPDYHGQDSVRVGRISFGSINAILGS |
| | | VALILNSHHHASAVAAAHHSEDPSSKAPKAPEVQLVESGGGLVQAGG |
| | | SLRLSCAASGITFSINTMGWYRQAPGKQRELVALISSIGDTYYADSV |
| | | KGRFTISRDNAKNTVYLQMNSLKPEDTAVYYCKRFRTAAQGTDYWGQ |
| | | GTQVTVSSA |
| HER2- | 105 | KEFTLDFSTAKTYVDSLNVIRSAIGTPLQTISSGGTSLLMIDSGIGD |
| targeting | | NLFAVDILGFDFTLGRFNNLRLIVERNNLYVTGFVNRTNNVFYRFAD |
| molecule #9 | | FSHVTFPGTTAVTLSADSSYTTLQRVAGISRTGMQINRHSLTTSYLD |
| | | LMSHSGTSLTQSVARAMLRFVTVTAEALRFRQIQRGFRTTLDDLSGA |
| | | SYVMTAEDVDLTLNWGRLSSVLPDYHGQDSVRVGRISFGSINAILGS |
| | | VALILNSHHHASAVAAEFPKPSTPPGSSGGAPQVQLQQSGPELKKPG |
| | | ETVKISCKASGYPFTNYGMNWVKQAPGQGLKWMGWINTSTGESTFAD |
| | | DFKGRFDFSLETSANTAYLQINNLKSEDSATYFCARWEVYHGYVPYW |
| | | GQGTTVTVSSGGGGSDIQMTQSPSSLSASVGDRVTITCKASQDVYNA |
| | | VAWYQQKPGQSPKLLIYSASSRYTGVPSRFTGSGSGPDFTFTISSVQ |
| | | AEDLAVYFCQQHFRTPFTFGSGTKLEIK |
| HER2- | 106 | KEFTLDFSTAKTYVDSLNVIRSAIGTPLQTISSGGTSLLMIDSGIGD |
| targeting | | NLFAVDILGFDFTLGRFNNLRLIVERNNLYVTGFVNRTNNVFYRFAD |
| molecule | | FSHVTFPGTTAVTLSADSSYTTLQRVAGISRTGMQINRHSLTTSYLD |
| #10 | | LMSHSGTSLTQSVARAMLRFVTVTAEALRFRQIQRGFRTTLDDLSGA |
| | | SYVMTAEDVDLTLNWGRLSSVLPDYHGQDSVRVGRISFGSINAILGS |
| | | VALILNSHHHASAVAAEVQLVESGGGLVQAGGSLRLSCAASGITFSI |
| | | NTMGWYRQAPGKQRELVALISSIGDTYYADSVKGRFTISRDNAKNTV |
| | | YLQMNSLKPEDTAVYYCKRFRTAAQGTDYWGQGTQVTVSSA |
| HER2- | 107 | KEFTLDFSTAKTYVDSLNVIRSAIGTPLQTISSGGTSLLMIDSGIGD |
| targeting | | NLFAVDILGFDFTLGRFNNLRLIVERNNLYVTGFVNRTNNVFYRFAD |
| molecule | | FSHVTFPGTTAVTLSADSSYTTLQRVAGISRTGMQINRHSLTTSYLA |
| #11 | | LMSHSGTSLTQSVARAMLRFVTVTAEALRFRQIQRGFRTTLDDLSGA |
| | | SYVMTAEDVDLTLNWGRLSSVLPDYHGQDSVRVGRISFGSINAILGS |
| | | VALILNSHHHASAVAAQVQLQESGGGSVQAGGSLKLTCAASGYIFNS |
| | | CGMGWYRQSPGRERELVSRISGDGDTWHKESVKGRFTISQDNVKKTL |
| | | YLQMNSLKPEDTAVYFCAVCYNLETYWGQGTQVTVSS |
| HER2- | 108 | KEFTLDFSTAKTYVDSLNVIRSAIGTPLQTISSGGTSLLMIDSGIGD |
| targeting | | NLFAVDILGFDFTLGRFNNLRLIVERNNLYVTGFVNRTNNVFYRFAD |
| molecule | | FSHVTFPGTTAVTLSADSSYTTLQRVAGISRTGMQINRHSLTTSYLD |
| #12 | | LMSHSGTSLTQSVARAMLRFVTVTAEALRFRQIQRGFRTTLDDLSGA |
| | | SYVMTAEDVDLTLNWGRLSSVLPDYHGQDSVRVGRISFGSINAILGS |
| | | VALILNSHHHASAVAAEFPKPSTPPGSSGGAPQVQLQQSGPELKKPG |
| | | ETVKISCKASGYPFTNYGMNWVKQAPGQGLKWMGWINTSTGESTFAD |
| | | DFKGRFDFSLETSANTAYLQINNLKSEDSATYFCARWEVYHGYVPYW |
| | | GQGTTVTVSSGGGGSGGGGSGGGGSGGGGSDIQMTQSPSSLSASVGD |
| | | RVTITCKASQDVYNAVAWYQQKPGQSPKLLIYSASSRYTGVPSRFTG |
| | | SGSGPDFTFTISSVQAEDLAVYFCQQHFRTPFTFGSGTKLEIK |

In some embodiments, a binding molecule comprises a binding region linker which links the Shiga toxin A subunit effector polypeptide and the binding region. For example, in some embodiments, the binding molecule comprises a binding region linker that links (i) the Shiga toxin subunit effector polypeptide and (ii) the VH or (iii) the VL. In some embodiments, the binding molecule comprises a scFv linker that links (ii) the VH and (iii) the VL. In some embodiments, the binding region linker comprises or consists of the sequence SSGGGGSGGGGSGGGGSGGGGSGGGGS (SEQ ID NO: 199). In some embodiments, the scFv linker comprises or consists of the sequence GSGSG (SEQ ID NO: 200). In some embodiments, the scFv linker comprises or consists of the sequence GGGGS (SEQ ID NO: 217). In some embodiments, the scFv linker comprises or consists of the sequence GGGGSGGGGSGGGGSGGGGSGGGGS (SEQ ID NO: 214).

ScFv linkers of variable length can be used in the binding molecules described herein. In some embodiments, linkers of 1 to 50 amino acids in length are used. In some embodiments, a linker of 3 to 12 amino acids in length is used, and the resulting scFv monomers tend to form multimers due to self-association, with the majority form being dimers. In some embodiments, linkers of 5 amino acids in length is used. In some embodiments, linkers of longer than 12 (e.g., 13, 14, 15, 16, 17, 18, 19, or 20) amino acids in length is used, and the resulting scFv predominantly forms monomers with only a minority fraction undergoing spontaneous multimerization.

In some embodiments, the binding molecule comprises, from N-terminus to C-terminus or from C-terminus to N-terminus, the Shiga toxin A subunit effector polypeptide, the binding region linker, and the binding region. In some embodiments, the binding molecule comprises, from N-terminus to C-terminus or from C-terminus to N-terminus, the Shiga toxin A subunit effector polypeptide, the binding region linker, the VH, and the VL. In some embodiments, the binding molecule comprises, from N-terminus to C-terminus or from C-terminus to N-terminus, the Shiga toxin A subunit effector polypeptide, the binding region linker, the VL, and the VH.

In some embodiments, the binding molecule comprises, from N-terminus to C-terminus or from C-terminus to N-terminus, the Shiga toxin A subunit effector polypeptide, the binding region linker, the VH, the scFv linker, and the VL. In some embodiments, the binding molecule comprises, from N-terminus to C-terminus or from C-terminus to N-terminus, the Shiga toxin A subunit effector polypeptide, the binding region linker, the VL, the scFv linker, and the VH.

Suitable linkers, whether proteinaceous or non-proteinaceous, can include, e.g., protease sensitive, environmental redox potential sensitive, pH sensitive, acid cleavable, photocleavable, and/or heat sensitive linkers.

Proteinaceous linkers can be chosen for incorporation into recombinant binding molecules. For recombinant binding molecules, linkers typically comprise about 2 to 50 amino acid residues, preferably about 5 to 30 amino acid residues. Commonly, proteinaceous linkers comprise a majority of amino acid residues with polar, uncharged, and/or charged residues, such as, e.g., threonine, proline, glutamine, glycine, and alanine. Non-limiting examples of proteinaceous linkers include alanine-serine-glycine-glycine-proline-glutamate (ASGGPE, SEQ ID NO: 201), valine-methionine (VM), alanine-methionine (AM), AM(G_{2 to 4}S)xAM where G is glycine, S is serine, and x is an integer from 1 to 10 (SEQ ID NO: 202).

Proteinaceous linkers can be selected based upon the properties desired. Proteinaceous linkers can be chosen by the skilled worker with specific features in mind, such as to optimize the binding molecule's folding, stability, expression, solubility, pharmacokinetic properties, pharmacodynamic properties, and/or the activity of the fused domains in the context of a fusion construct as compared to the activity of the same domain by itself. For example, proteinaceous linkers can be selected based on flexibility, rigidity, and/or cleavability. The skilled worker can use databases and linker design software tools when choosing linkers. In certain linkers can be chosen to optimize expression. In certain linkers can be chosen to promote intermolecular interactions between identical polypeptides or proteins to form homomultimers or different polypeptides or proteins to form heteromultimers. For example, proteinaceous linkers can be selected which allow for desired non-covalent interactions between polypeptide components of the binding molecules, such as, e.g., interactions related to the formation dimers and other higher order multimers.

Flexible proteinaceous linkers are often greater than 12 amino acid residues long and rich in small, non-polar amino acid residues, polar amino acid residues, and/or hydrophilic amino acid residues, such as, e.g., glycines, serines, and threonines. Flexible proteinaceous linkers can be chosen to increase the spatial separation between components and/or to allow for intramolecular interactions between components. For example, various “GS” linkers are known to the skilled worker and are composed of multiple glycines and/or serines, sometimes in repeating units, such as, e.g., (GxS)ₙ, (SEQ ID NO: 203), (SxG)ₙ (SEQ ID NO: 204), (GGGGS)ₙ (SEQ ID NO: 205), and (G)ₙ (SEQ ID NO: 206), in which x is 1 to 6 and n is 1 to 30. Non-limiting examples of flexible proteinaceous linkers include GKSSGSGSESKS (SEQ ID NO: 207), EGKSSGSGSESKEF (SEQ ID NO: 208), GSTSGSGKSSEGKG (SEQ ID NO: 209), GSTSGSGKSSEGSGSTKG (SEQ ID NO: 210), GSTSGSGKPGSGEGSTKG (SEQ ID NO: 211), SRSSG (SEQ ID NO: 212), and SGSSC (SEQ ID NO: 213).

Rigid proteinaceous linkers are often stiff alpha-helical structures and rich in proline residues and/or strategically placed prolines. Rigid linkers can be chosen to prevent intramolecular interactions between linked components.

Additional examples of suitable linkers are provided in Table 7.

TABLE 7
Linkers

| Linker | | SEQ |
|---|---|---|
| name | Sequence | ID NO |
| linker 1 | GGGGSGGGGSGGGGSGGGGSGGG | 214 |
| | GS | |
| linker 2 | GGGGSGGGGSGGGGSGGGGS | 215 |
| linker 3 | GSTSGSGKPGSGEGSTKG | 216 |
| linker 4 | GGGGS | 217 |
| linker 5 | EFPKPSTPPGSSGGAP | 218 |
| linker 6 | EFPKPSTPPGSSGGAPGILGFVFTL | 219 |
| linker 7 | GSTSGSGKPGSGEGS | 220 |
| linker 8 | SPSTPPTPSPSTPPAS | 221 |
| linker 9 | AHHSEDPSSKAPKAP | 222 |

Suitable linkers can allow for in vivo separation of components, such as, e.g., due to cleavage and/or environment-specific instability. In vivo cleavable proteinaceous linkers are capable of unlinking by proteolytic processing and/or reducing environments often at a specific site within an organism or inside a certain cell type. In vivo cleavable proteinaceous linkers often comprise protease sensitive motifs and/or disulfide bonds formed by cysteine pairs. In vivo cleavable proteinaceous linkers can be designed to be sensitive to proteases that exist only at certain locations in an organism, compartments within a cell, and/or become active only under certain physiological or pathological conditions (such as, e.g., involving proteases with abnormally high levels, proteases overexpressed at certain disease sites, and proteases specifically expressed by a pathogenic microorganism). For example, there are proteinaceous linkers known in the art which are cleaved by proteases present only intracellularly, proteases present only within specific cell types, and proteases present only under pathological conditions like cancer or inflammation, such as, e.g., R-x-x-R motif (SEQ ID NO: 195) and AMGRSGGGCAGNRVGSSLSCGGLNLQAM (SEQ ID NO: 223).

In some embodiments, a linker can comprise a protease sensitive site to provide for cleavage by a protease present within a target cell. In some embodiments, the linker is not cleavable, so as to reduce unwanted toxicity after administration to a vertebrate organism.

Suitable linkers include, e.g., protease sensitive, environmental redox potential sensitive, pH sensitive, acid cleavable, photocleavable, and/or heat sensitive linkers, whether proteinaceous or non-proteinaceous. Suitable cleavable linkers can include linkers comprising cleavable groups which are known in the art.

Suitable linkers can include pH sensitive linkers. For example, certain suitable linkers can be chosen for their instability in lower pH environments to provide for dissociation inside a subcellular compartment of a target cell. For example, linkers that comprise trityl groups, derivatized trityl groups, bismaleimideothoxy propane groups, adipic acid dihydrazide groups, and/or acid labile transferrin groups, can provide for release of components of the binding molecules, e.g. a polypeptide component, in environments with specific pH ranges. In certain linkers can be chosen which are cleaved in pH ranges corresponding to physiological pH differences between tissues.

Photocleavable linkers are linkers that are cleaved upon exposure to electromagnetic radiation of certain wavelength ranges, such as light in the visible range. Photocleavable linkers can be used to release a component of a binding molecule, e.g. a polypeptide component, upon exposure to light of certain wavelengths. Non-limiting examples of photocleavable linkers include a nitrobenzyl group as a photocleavable protective group for cysteine, nitrobenzyloxycarbonyl chloride cross-linkers, hydroxypropylmethacrylamide copolymer, glycine copolymer, fluorescein copolymer, and methylrhodamine copolymer. Photocleavable linkers can have particular uses in linking components to form binding molecules designed for treating diseases, disorders, and conditions that can be exposed to light using fiber optics.

In some embodiments, the binding molecules are monomers. In some embodiments, the binding proteins are dimers, such as homodimers or heterodimers. In some embodiments, the binding proteins are homodimers comprising two identical polypeptides. In some embodiments, the binding proteins are multimers comprising, for example, two, three, four, five, six, seven, eight, nine, ten, or more binding polypeptides.

### Compositions

Also provided herein are compositions comprising one or more HER2 binding molecules of the disclosure. In some embodiments, the compositions are pharmaceutical compositions. In some embodiments, the compositions are useful for treatment or prophylaxis of a HER2-positive cancer, or conditions, diseases, or symptoms associated therewith.

Pharmaceutical compositions comprising a binding molecule, or an acceptable salt or solvate thereof, can also comprise a pharmaceutically acceptable carrier, excipient, surfactant, stabilizer, antioxidant, vehicle, etc. Such agents should be non-toxic and should not interfere with the stability or efficacy of the binding molecule. Illustrative pharmaceutically acceptable buffers include histidine-buffers, citrate-buffers, succinate-buffers, acetate-buffers and phosphate-buffers or mixtures thereof. Exemplary stabilizing agents include sugars or sugar alcohols (e.g., mannitol, dextrose, glucose, trehalose, and/or sucrose). Inorganic salts (e.g., sodium chloride (NaCl), sodium sulfate (Na2SO4), sodium thiocyanate (NaSCN), magnesium chloride (MgCl), magnesium sulfate (MgSO4), ammonium thiocyanate (NH4SCN), ammonium sulfate ((NH4)2SO4), ammonium chloride (NH4Cl), calcium chloride (CaCl2), calcium sulfate (CaSO4), zinc chloride (ZnCl2)) may also be used as stabilizers. Illustrative surfactants include oloxamers, polysorbates, polyoxy ethylene alkyl ethers (Brij), alkylphenylpolyoxyethylene ethers (Triton-X) or sodium dodecyl sulphate (SDS). Suitable tonicity agents include but are not limited to salts, amino acids and sugars (e.g., sodium chloride, trehalose, sucrose or arginine). Antioxidants include but are not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. Chelating agents, reactive oxygen scavengers and chain terminators can also be used. Additional suitable carriers, diluents, excipients, stabilizers, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994. The precise nature of the carrier or other agent will depend on the route of administration, which may be oral, or by injection, e.g. cutaneous, subcutaneous, or intravenous.

In some embodiments, the compositions comprising binding molecules described herein are useful for intravenous infusion. In some embodiments, the binding molecules are formulated in an aqueous buffer solution containing a cryogenic protectant and a surfactant.

Pharmaceutical compositions can conveniently be presented in unit dosage form and can be prepared by any of the methods well known in the art of pharmacy. In such form, the composition is divided into unit doses containing appropriate quantities of the active component. Compositions can be formulated for any suitable route and means of administration.

In some embodiments, a pharmaceutical composition comprising a binding molecule (i.e., a HER2 binding molecule) as described herein, and at least one pharmaceutically acceptable excipient or carrier. In some embodiments, a pharmaceutical composition comprises: a binding molecule comprising a (i) Shiga toxin A subunit effector polypeptide and (ii) a binding region capable of specifically binding HER2 and (iii) a pharmaceutically acceptable carrier, excipient or buffer. In some embodiments, a pharmaceutical composition comprises: a binding molecule comprising a (i) Shiga toxin A subunit effector polypeptide and (ii) a binding region capable of specifically binding a target on the surface of an immune cell; and (iii) a pharmaceutically acceptable carrier, excipient or buffer.

In some embodiments, a composition comprising a HER2 binding molecule (e.g., 115111) comprises a formulation buffer comprising one or more of sodium citrate, citric acid, sorbitol, and polysorbate 20. In some embodiments, the formulation buffer comprises sodium citrate, citric acid, sorbitol, and polysorbate 20. In some embodiments, the formulation buffer comprises sodium citrate, sorbitol, and polysorbate 20. The concentration of HER2 binding molecule in the composition may be about 0.1 mg/mL to about 1 mg/mL, for example about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL or about 1.0 mg/mL. In some embodiments, the concentration of HER2 binding molecule is about 0.5 mg/mL.

In some embodiments, the formulation buffer comprises sodium citrate at a concentration of about 0.5 mg/mL to about 10 mg/mL, for example about 0.5 mg/mL, about 1.0 mg/mL, about 1.5 mg/mL, about 2.0 mg/mL, about 2.5 mg/mL, about 3.0 mg/mL, about 3.5 mg/mL, about 4.0 mg/mL, about 4.5 mg/mL, about 5.0 mg/mL, about 5.5 mg/mL, about 6.0 mg/mL, about 6.5 mg/mL, about 7.0 mg/mL, about 7.5 mg/mL, about 8.0 mg/mL, about 8.5 mg/mL, about 9.0 mg/mL, about 9.5 mg/mL, or about 10.0 mg/mL. In some embodiments, the formulation buffer comprises about 4.5 mg/mL sodium citrate.

In some embodiments, the formulation buffer comprises sodium citrate at a concentration of about 0.5 mM to about 50 mM. For example, in some embodiments, the formulation buffer comprises sodium citrate at a concentration of about 0.5 mM, about 1 mM, about 5 mM, about 10 mM, about 15 mM, about 20 mM, about 25 mM, about 30 mM, about 35 mM, about 40 mM, about 45 mM, about 50 mM, or any range or value therebetween. In some embodiments, the concentration of sodium citrate is about 20 mM.

In some embodiments, the formulation buffer comprises citric acid at a concentration of about 0.1 mg/mL to about 2.0 mg/mL, for example about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, or about 2.0 mg/mL. In some embodiments, the concentration of citric acid is about 1.0 mg/mL.

In some embodiments, the formulation buffer comprises sorbitol at a concentration of about 1 mg/mL to about 100 mg/mL, for example, about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL. In some embodiments, the concentration of sorbitol is about 30 mg/mL to about 40 mg/mL, such as about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, or about 40 mg/mL. In some embodiments, the concentration of sorbitol is about 36.4 mg/mL.

In some embodiments, the formulation buffer comprises sorbitol at a concentration of about 50 mM to about 500 mM. For example, in some embodiments, the formulation buffer comprises sorbitol at a concentration of about 50 mM, about 75 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, about 500 mM, or any range or value therebetween. In some embodiments, the concentration of sorbitol is about 200 mM.

In some embodiments, the formulation buffer comprises polysorbate 20 at a concentration of about 0.001% (v/v) to about 0.1% (v/v). For example, in some embodiments, the formulation buffer comprises polysorbate 20 at a concentration of about 0.001% (v/v), about 0.005% (v/v), about 0.01% (v/v), about 0.02% (v/v), about 0.03% (v/v), about 0.04% (v/v), about 0.05% (v/v), about 0.06% (v/v), about 0.07% (v/v), about 0.08% (v/v), about 0.09% (v/v), or about 0.1% (v/v). In some embodiments, the concentration of polysorbate 20 is about 0.02% (v/v).

In some embodiments, the formulation buffer has a pH in the range of about 4.0 to about 7.0. For example, in some embodiments, the pH of the formulation buffer is about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0. In some embodiments, the pH of the formulation buffer is about 5.3 to about 5.7. In some embodiments, the pH of the formulation buffer is about 5.5. In some embodiments, the pH of the buffer is adjusted with, for example, sodium hydroxide and/or hydrochloric acid, as needed.

In some embodiments, a composition comprises: (i) about 0.1 mg/mL to about 1 mg/mL of a HER2 binding molecule, (ii) about 0.5 mg/mL to about 10 mg/mL sodium citrate, (iii) about 1 mg/mL to about 100 mg/mL sorbitol, (iv) about 0.001% (v/v) to about 0.1% (v/v) polysorbate 20, and has a pH of about 5.3 to about 5.7. In some embodiments, a composition comprises: (i) about 0.5 mg/mL of a HER2 binding molecule, (ii) about 5.2 mg/mL sodium citrate, (iii) about 36.4 mg/mL sorbitol, (iv) about 0.02% (v/v) polysorbate 20, and has a pH of about 5.5.

In some embodiments, a composition comprises: (i) about 0.1 mg/mL to about 1 mg/mL of a HER2 binding molecule, (ii) about 0.5 mM to about 50 mM sodium citrate, (iii) about 50 mM to about 500 mM sorbitol, (iv) about 0.001% (v/v) to about 0.1% (v/v) polysorbate 20, and has a pH of about 5.3 to about 5.7. In some embodiments, a composition comprises: (i) about 0.5 mg/mL of a HER2 binding molecule, (ii) about 20 mM sodium citrate, (iii) about 200 mM sorbitol, (iv) about 0.02% (v/v) polysorbate 20, and has a pH of about 5.5.

In some embodiments, a composition comprises: (i) about 0.1 mg/mL to about 1 mg/mL of a 115111 molecule, (ii) about 0.5 mg/mL to about 10 mg/mL sodium citrate, (iii) about 1 mg/mL to about 100 mg/mL sorbitol, (iv) about 0.001% (v/v) to about 0.1% (v/v) polysorbate 20, and has a pH of about 5.3 to about 5.7. In some embodiments, a composition comprises: (i) about 0.5 mg/mL of 115111 molecule, (ii) about 5.2 mg/mL sodium citrate, (iii) about 36.4 mg/mL sorbitol, (iv) about 0.02% (v/v) polysorbate 20, and has a pH of about 5.5.

In some embodiments, a composition comprises: (i) about 0.1 mg/mL to about 1 mg/mL of a 115111 molecule, (ii) about 0.5 mM to about 50 mM sodium citrate, (iii) about 50 mM to about 500 mM sorbitol, (iv) about 0.001% (v/v) to about 0.1% (v/v) polysorbate 20, and has a pH of about 5.3 to about 5.7. In some embodiments, a composition comprises: (i) about 0.5 mg/mL of a 115111 molecule, (ii) about 20 mM sodium citrate, (iii) about 200 mM sorbitol, (iv) about 0.02% (v/v) polysorbate 20, and has a pH of about 5.5.

Diagnostic compositions can comprise a binding molecule and at least one detection promoting agent. When producing or manufacturing a diagnostic composition, a binding molecule can be directly or indirectly linked to at least one detection promoting agent. There are numerous standard techniques known to the skilled worker for incorporating, affixing, and/or conjugating various detection promoting agents to proteins or proteinaceous components of molecules, especially to immunoglobulins and immunoglobulin-derived domains.

There are numerous detection promoting agents known to the skilled worker, such as isotopes, dyes, colorimetric agents, contrast enhancing agents, fluorescent agents, bioluminescent agents, and magnetic agents, which can be operably linked to the polypeptides or binding molecules for information gathering methods, such as for diagnostic and/or prognostic applications to diseases or conditions of an organism. The incorporation of the agent is in such a way to enable the detection of the presence of the diagnostic composition in a screen, assay, diagnostic procedure, and/or imaging technique.

Similarly, there are numerous imaging approaches known to the skilled worker, such as non-invasive in vivo imaging techniques commonly used in the medical arena, for example: computed tomography imaging (CT scanning), optical imaging (including direct, fluorescent, and bioluminescent imaging), magnetic resonance imaging (MRI), positron emission tomography (PET), single-photon emission computed tomography (SPECT), ultrasound, and x-ray computed tomography imaging.

### Methods of Treatment

Also provided herein are methods for treating a subject in need thereof, the methods comprising administering to the subject an effective amount of (i) a binding molecule (e.g., a HER2 binding molecule), (ii) a nucleic acid encoding the binding molecule, or (iii) a composition comprising a binding molecule or nucleic acid encoding the same.

As used herein, the term “subject” refers to any organism, commonly a mammalian subject, such as a human or non-human animal. The terms “subject” and “patient” are used interchangeably. In some embodiments, the subject can be a mammal, such as a primate (e.g., a human or non-human primate), a livestock animal (e.g., cow, horse, pig, sheep, goat, etc.), a companion animal (e.g., cat, dog, etc.) or a laboratory animal (e.g., mouse, rabbit, rat, etc.). In some embodiments, the subject presents one or more symptoms, signs, and/or indications of cancer, such as a HER2-positive cancer.

As used herein, the terms “treat,” “treating,” or “treatment”, and grammatical variants thereof, have the same meaning as commonly understood by those of ordinary skill in the art. In some embodiments, these terms can refer to an approach for obtaining beneficial or desired clinical results. The terms can refer to slowing the onset or rate of development of a condition, disorder or disease, reducing or alleviating symptoms associated with it, generating a complete or partial regression of the condition, or some combination of any of the above. As described herein, beneficial or desired clinical results include, but are not limited to, reduction or alleviation of symptoms, diminishment of extent of disease, stabilization (e.g., not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject (e.g., a human) in need of treatment can thus be a subject already afflicted with the disease or disorder in question. The terms “treat,” “treating,” or “treatment” includes inhibition or reduction of an increase in severity of a pathological state or symptoms relative to the absence of treatment, and is not necessarily meant to imply complete cessation of the relevant disease or condition.

As used herein, the terms “prevent,” “preventing,” “prevention” and grammatical variants thereof refer to an approach for preventing the development of, or altering the pathology of, a condition or disease. Accordingly, “prevention” can refer to prophylactic or preventive measures. As described herein, beneficial or desired clinical results include, but are not limited to, prevention or slowing of symptoms, progression or development of a disease, whether detectable or undetectable. A subject (e.g. a human) in need of prevention can thus be a subject not yet afflicted with the disease or disorder in question. The term “prevention” includes slowing the onset of disease relative to the absence of treatment and is not necessarily meant to imply permanent prevention of the relevant disease, disorder or condition. Thus “preventing” or “prevention” of a condition can in certain contexts refer to reducing the risk of developing the condition, or preventing or delaying the development of symptoms associated with the condition.

In some embodiments, a method of treating or preventing cancer, the method comprising administering to a subject in need thereof an effective amount of a HER2 binding molecule comprising: (A) a cytotoxic Shiga toxin A subunit effector polypeptide; and (B) a binding region capable of specifically binding an extracellular part of human HER2, wherein the binding region comprises: (a) an immunoglobulin heavy chain variable region comprising: a CDR1 comprising the sequence of SEQ ID NO: 57; a CDR2 comprising the sequence of SEQ ID NO: 58; and a CDR3 comprising the sequence of SEQ ID NO: 59; and (b) an immunoglobulin light chain variable region comprising: a CDR1 comprising the sequence of SEQ ID NO: 60; a CDR2 comprising the sequence of SEQ ID NO: 61; and a CDR3 comprising the sequence of SEQ ID NO: 62; wherein the effective amount is a dose in the range of about 0.1 to about 15 μg/kg.

The dose of binding molecule administered to the subject may be any amount effective for treating and/or preventing cancer, or a sign or symptom thereof. In some embodiments, the effective amount is a dose in the range of about 0.001 μg/kg to 1000 μg/kg, such as about 0.01 μg/kg to about 500 μg/kg, about 0.01 μg/kg to about 300 μg/kg, about 0.01 μg/kg to about 100 μg/kg, about 0.1 μg/kg to about 100 μg/kg, about 0.1 μg/kg to about 75 μg/kg, about 0.1 μg/kg to about 50 μg/kg, or about 0.1 μg/kg to about 15 μg/kg. In some embodiments, the effective amount is a dose in the range of about 12.5 μg/kg to about 15.0 μg/kg, 15.6 μg/kg to about 22.5 μg/kg, about 19.5 μg/kg to about 33.75 μg/kg, about 24.4 μg/kg to about 50.6 μg/kg, about 30.5 μg/kg to about 75.9 μg/kg, or about 38.1 μg/kg to about 113.9 μg/kg.

In some embodiments, the dose is about 0.5 μg/kg, about 1.0 μg/kg, about 2.0 μg/kg, about 3.0 μg/kg, about 4.5 μg/kg, about 6.75 μg/kg, or about 10.0 μg/kg. In some embodiments, the dose is about 12.5 μg/kg, about 15.0 μg/kg, about 15.6 μg/kg, about 19.5 μg/kg, about 22.5 μg/kg, about 24.4 μg/kg, about 25.0 μg/kg, about 30.5 μg/kg, about 33.25 μg/kg, about 33.75 μg/kg, about 44.2 μg/kg, about 50.6 μg/kg, about 58.8 μg/kg, about 75.9 μg/kg, about 78.2 μg/kg, about 104 μg/kg, or about 113.9 μg/kg. In some embodiments, the dose is about 1 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, about 225 μg/kg, about 250 μg/kg, about 275 μg/kg, or about 300 μg/kg.

The binding molecule may be administered all at once as a single bolus dose or may be administered to the subject over a period of time. For example, in some embodiments, the binding molecule may be administered to the subject over a period of about 1 hour to about 12 hours, about 1 hour to about 4 hours, about 30 minutes to about 2 hours, or 10 minutes to about 1 hour. In some embodiments, the binding molecule is administered to the subject over a period of about 20 minutes, about 30 minutes, or about 40 minutes. In some embodiments, the binding molecule is administered to the subject over a period of about 30 minutes.

In some embodiments, the HER2 binding molecule is administered to the subject once. In some embodiments, the HER2 binding molecule is administered to the subject more than once, such as two, three, four, five, six, seven, eight, nine, ten times. In some embodiments, the HER2 binding molecule is administered to the subject more than ten times.

The binding molecule may be administered at therapeutic intervals. For example, the binding molecule may be administered once per day, once per week, twice per week, twice per month, three times per month, once per month, once every two months, once every three months, one every four months, once every five months, once every six months, or once per year.

In some embodiments, the HER2 binding molecule is administered to the subject every seven days. For example, the HER2 binding molecule may be administered to the subject on days 1, 8, and 15, wherein day 1 indicates the first day of the treatment cycle. In some embodiments, the treatment cycle is 21 days.

In some embodiments, the subject is administered a dose in the range of about 0.1 μg/kg to about 50 μg/kg at each administration. For example, in some embodiments, the subject is administered a dose of about 0.5 μg/kg, about 1.0 μg/kg, about 2.0 μg/kg, about 3.0 μg/kg, about 4.5 μg/kg, about 6.75 μg/kg, or about 10.0 μg/kg at each administration. In some embodiments, the subject is administered a dose of about 12.5 μg/kg, about 15.0 μg/kg, about 15.6 μg/kg, about 19.5 μg/kg, about 22.5 μg/kg, or about 33.75 μg/kg.

In some embodiments, the subject is administered 0.5 μg/kg of the binding molecule on days 1, 8, and 15 of a 21 day cycle. In some embodiments, the subject is administered 1.0 μg/kg of the binding molecule on days 1, 8, and 15 of a 21 day cycle. In some embodiments, the subject is administered 2.0 μg/kg of the binding molecule on days 1, 8, and 15 of a 21 day cycle. In some embodiments, the subject is administered 3.0 μg/kg of the binding molecule on days 1, 8, and 15 of a 21 day cycle. In some embodiments, the subject is administered 4.5 μg/kg of the binding molecule on days 1, 8, and 15 of a 21 day cycle. In some embodiments, the subject is administered 6.75 μg/kg of the HER2 binding molecule on days 1, 8, and 15 of a 21 day cycle. In some embodiments, the subject is administered 10.0 μg/kg of the HER2 binding molecule on days 1, 8, and 15 of a 21 day cycle. In some embodiments, the subject is administered 12.5 μg/kg of the HER2 binding molecule on days 1, 8, and 15 of a 21 day cycle. In some embodiments, the subject is administered 15.0 μg/kg of the HER2 binding molecule on days 1, 8, and 15 of a 21 day cycle. In some embodiments, the subject is administered 15.6 μg/kg of the HER2 binding molecule on days 1, 8, and 15 of a 21 day cycle. In some embodiments, the subject is administered 19.5 μg/kg of the HER2 binding molecule on days 1, 8, and 15 of a 21 day cycle. In some embodiments, the subject is administered 22.5 μg/kg of the HER2 binding molecule on days 1, 8, and 15 of a 21 day cycle. In some embodiments, the subject is administered 33.75 μg/kg of the HER2 binding molecule on days 1, 8, and 15 of a 21 day cycle.

In some embodiments, a method of killing a HER2-positive cell comprises the step of contacting the cell with a binding molecule or a pharmaceutical composition as described herein. In some embodiments, the binding molecule is cytotoxic.

In some embodiments, a method of treating cancer (e.g., a HER2-positive cancer) comprises administering to a subject in need thereof an effective amount of a binding molecule or a pharmaceutical composition as described herein.

In some embodiments, a method for treating cancer (e.g., a HER2-positive cancer) comprises administering to the subject in need thereof an effective amount a nucleic acid or an expression vector as described herein, e.g., a nucleic acid or an expression vector encoding a HER2 binding molecule or a fragment or variant thereof.

In some embodiments, the binding molecule binds to HER2, but does not block the interaction between HER2 and one or more of its ligands. For example, in some embodiments, a HER2 binding molecule does not block the interaction between HER2 and one or more of its epidermal growth factor-like ligands.

In some embodiments, the binding molecule binds to HER2 and also blocks the interaction between HER2 and one or more of its ligands. For example, in some embodiments, a HER2 binding molecule blocks the interaction between HER2 and one or more of its epidermal growth factor-like ligands.

In some embodiments, the subject has a HER2-positive cancer. In some embodiments, the HER2-positive cancer is relapsed or refractory to one or more additional therapies.

Administration of the HER2 binding molecule may be by any acceptable route. For example, the HER2 binding molecule may be administered to the subject by intravenous, subcutaneous, or intramuscular injection. In some embodiments, the HER2 binding molecule is administered to the subject by intravenous injection. In some embodiments, the HER2 composition is administered intramuscularly, intravenously, subcutaneously, intranasally, or intraperiotoneally.

The binding molecules or pharmaceutical compositions described herein can be administered alone or in combination with other therapeutic or diagnostic agents. A combination therapy can include a binding molecule, or pharmaceutical composition thereof, combined with at least one other therapeutic agent selected based on the particular subject, disease or condition to be treated. Examples of other such agents include, inter alia, a cytotoxic, anti-cancer or chemotherapeutic agent, a checkpoint inhibitor, an anti-inflammatory or anti-proliferative agent, an antimicrobial or antiviral agent, growth factors, cytokines, an analgesic, a therapeutically active small molecule or polypeptide, a single chain antibody, a classical antibody or fragment thereof, or a nucleic acid molecule which modulates signaling pathways, and similar modulating therapeutic molecules which can complement or otherwise be beneficial in a therapeutic or prophylactic treatment regimen.

In some embodiments, treatment of a subject with a binding molecule or pharmaceutical composition leads to cell death of targeted cells and/or the inhibition of growth of targeted cells. The targeted cells may be, for example, HER2-positive cells.

The HER2 binding molecule may be administered as a part of a combination therapy. For example, in some embodiments, a method of treating a subject may comprise administering to the subject the HER2 binding molecule in combination with a second anti-cancer agent. The second anti-cancer agent may be, for example, a protein, a nucleic acid, or a small molecule. In some embodiments, the second anti-cancer agent is a second HER2 binding molecule, such as trastuzumab or pertuzumab. In some embodiments, the second anti-cancer agent is trastuzumab emtansine, tucatinib, or fam-trastuzumab deruxtecan. In some embodiments, the second anti-cancer agent is a chemotherapeutic agent such as docetaxel, capecitabine, fluorouracil, or cisplatin.

The subject to be treated with the HER2 molecules of the instant disclosure may have a disease, disorder, or condition involving HER2 expression and/or activity. For example, the subject may have cancer, such as a HER2-positive cancer. In some embodiments, the cancer is breast cancer or gastric or gastroesophageal adenocarcinoma, such as a HER2-positive breast cancer, or a HER2-positive gastric or gastroesophageal adenocarcinoma. In some embodiments, the cancer is cholangiocarcinoma, such as HER2-positive cholangiocarcinoma.

In some embodiments, the cancer involves a HER2 expressing cell. In some embodiments, the cancer involving a HER2 expressing cell is any one of the following cancers: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer, such as, e.g., breast cancer, gastric cancer (such as gastric adenocarcinoma), urothelial cancer (such as urothelial carcinoma), bladder cancer, urothelial bladder cancer, serous uterine cancer, extrahepatic biliary tract cancer, or biliary carcinoma. In some embodiments, the cancer involving a HER2 expressing cell is any one of the following cancers: bone cancer (such as multiple myeloma or Ewing's sarcoma), breast cancer, central/peripheral nervous system cancer (such as brain cancer, neurofibromatosis, or glioblastoma), gastrointestinal cancer (such as gastrointestinal stromal tumors, stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), gallbladder cancer, cholangiocarcinoma, head and neck cancer, liver cancer, lung/pleura cancer (such as mesothelioma, small cell lung carcinoma, or non-small cell lung carcinoma), prostate cancer, sarcoma (such as angiosarcoma, fibrosarcoma, Kaposi's sarcoma, or synovial sarcoma), skin cancer (such as basal cell carcinoma, squamous cell carcinoma, or melanoma), cervical cancer, and uterine cancer. In some embodiments, the cancer is a HER2-positive form of: an epithelial malignancy, breast cancer, gastric cancer, urothelial cancer, cholangiocarcinoma, gallbladder cancer, bladder cancer, urothelial bladder cancer, cervical cancer, testicular cancer, ovarian cancer, uterine cancer, serous uterine cancer, head and neck cancer, non-small cell lung cancer, colorectal cancer, extrahepatic biliary tract cancer, or biliary carcinoma.

In some embodiments, the cancer is a HER2-positive form of any one of the following cancers: bone cancer, breast cancer, central/peripheral nervous system cancer, gastrointestinal cancer, germ cell cancer, glandular cancer, head-neck cancer, hematological cancer, kidney-urinary tract cancer, gallbladder cancer, cholangiocarcinoma, head and neck cancer, liver cancer, lung/pleura cancer, prostate cancer, sarcoma, skin cancer, cervical cancer, and uterine cancer, such as, e.g., breast cancer, gastric cancer, urothelial cancer, bladder cancer, urothelial bladder cancer, serous uterine cancer, extrahepatic biliary tract cancer, or biliary carcinoma. In some embodiments, the cancer is HER2-positive breast cancer, gastric cancer, gastroesophageal adenocarcinoma, or cholangiocarcinoma.

In some embodiments, the cancer is a HER2-positive form of any one of the following cancers: bone cancer (such as multiple myeloma or Ewing's sarcoma), breast cancer, central/peripheral nervous system cancer (such as brain cancer, neurofibromatosis, or glioblastoma), gastrointestinal cancer (such as gastrointestinal stromal tumors, stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), gallbladder cancer, cholangiocarcinoma, liver cancer, lung/pleura cancer (such as mesothelioma, small cell lung carcinoma, or non-small cell lung carcinoma), prostate cancer, sarcoma (such as angiosarcoma, fibrosarcoma, Kaposi's sarcoma, or synovial sarcoma), skin cancer (such as basal cell carcinoma, squamous cell carcinoma, or melanoma), cervical cancer, and uterine cancer. In some embodiments, the cancer is a HER2-positive form of: an epithelial malignancy, breast cancer, gastric cancer, urothelial cancer, cholangiocarcinoma, gallbladder cancer, bladder cancer, urothelial bladder cancer, cervical cancer, testicular cancer, ovarian cancer, uterine cancer, serous uterine cancer, head and neck cancer, non-small cell lung cancer, colorectal cancer, extrahepatic biliary tract cancer, or biliary carcinoma.

In some embodiments, the subject has a cancer that is relapsed or refractory to at least one other cancer therapy, such as at least 2, 3, 4, 5, 6, 7, 8, 9 10, or more other cancer therapies. For example, in some embodiments, the cancer is relapsed or refractory to at least two prior lines of cancer therapy. In some embodiments, the subject is relapsed or refractory to trastuzumab or pertuzumab. In some embodiments, the subject is relapsed or refractory to trastuzumab emtansine, tucatinib, or fam-trastuzumab deruxtecan. In some embodiments, the subject is relapsed or refractory to a chemotherapeutic agent such as docetaxel, capecitabine, fluorouracil, or cisplatin.

In some embodiments, the subject has a previously treated advanced HER-2 positive solid tumor or cancer. In some embodiments, the cancer is breast cancer, gastroesophageal cancer or other solid cancer. In some examples, the cancer is breast cancer, gastric adenocarcinoma, gastroesophageal adenocarcinoma, urothelial carcinoma, non-small lung cancer, metastatic colorectal carcinoma or cholangiocarcinoma. In some embodiments, the cancer is a gynecological cancer. In some embodiments, the cancer is epithelial. In some embodiments, the cancer is bladder cancer, gallbladder cancer or cholangiocarcinoma.

In some embodiments, the subject is known to be intolerant to at least one other cancer therapy, such as at least 2, 3, 4, 5, 6, 7, 8, 9 10, or more other cancer therapies. For example, in some embodiments, the subject is known to be intolerant of at least two prior lines of cancer therapy.

### Kits

Also provided herein are kits comprising a binding molecule, and optionally, instructions for use, additional reagent(s), and/or pharmaceutical delivery device(s). The kit can comprise reagents and other tools for detecting a cell type (e.g., a HER2-positive cell) in a sample or in a subject.

In some embodiments, provided herein are a device comprising a binding molecule (e.g., in the form of a pharmaceutical composition or diagnostic composition), for delivery to a subject in need thereof. Thus, a delivery device comprising a composition as described herein can be used to administer to a subject a binding molecule by various delivery methods, including: intravenous, subcutaneous, intramuscular or intraperitoneal injection; or by other suitable means recognized by a person of skill in the art.

Also provided herein are kits comprising at least one composition of matter disclosed herein (e.g., a binding molecule), and optionally, packaging and instructions for use. Kits can be useful for drug administration and/or diagnostic information gathering. In some embodiments, a kit can optionally comprise at least one additional reagent (e.g., standards, markers and the like). Kits typically include a label indicating the intended use of the contents of the kit. The kit can further comprise reagents and other tools for detecting a cell type (e.g., a HER2-positive cell) in a sample or in a subject, or for diagnosing whether a subject belongs to a group that responds to a therapeutic strategy which makes use of a compound, composition, or related method, e.g., such as a method described herein.

### EXAMPLES

The present invention is further illustrated by the following non-limiting examples.

### Example 1: 115111 Activates Caspase Activity on Target Cells, Consistent with Delivery of the Shiga Toxin Subunit A Effector Peptide

Shiga and Shiga-like toxins are known to induce apoptotic cell death through activation of caspases. To determine whether 115111 can induce apoptosis in HER2-positive (HCC1954) and HER2 negative (MDA-MB-468) target cells, 115111 was added to the cells and the cells were incubated at 37° C., in a humidified, 5% C02 atmosphere. Caspase activity was measured 20 hours after addition of the 115111, using the Caspase 3/7-Glo® (Promgea®) method.

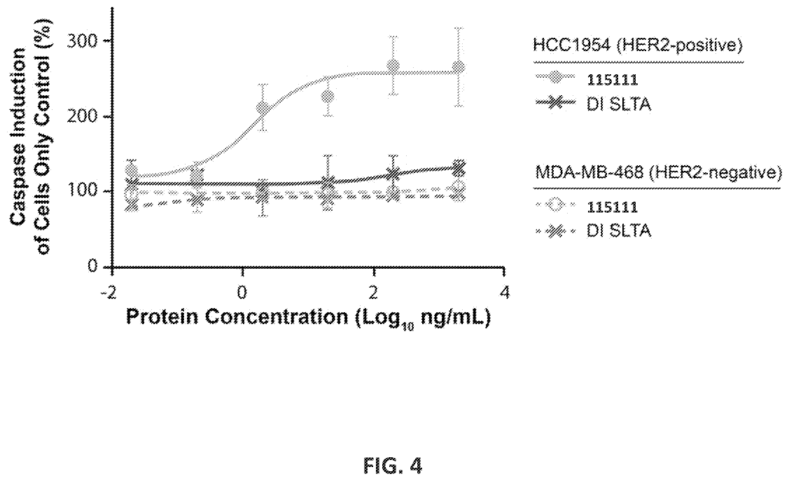

As shown in FIG. 4, 115111 induced caspase activation when incubated with HER2-positive HCC1954 cells. Caspase activation by 115111 was not observed for HER2 negative MDA-MB-468 cells. Caspase activation by the Shiga toxin A subunit effector peptide alone was not observed on either cell line.

### Example 2: 115111 has Potent and Specific Activity on HER2-Positive Cell Lines

To determine whether 115111 has cytotoxic activity on select cell lines, a cell line panel consisting of 47 distinct cell lines was evaluated for HER2 surface expression by flow cytometry and reported as HER2-specific monoclonal antibody-isotype control signal (S/I). The same panel was tested for cytotoxic activity by Cell Titer-Glo® (Promega®) of HER2-targeted agents, 115111 and ado-trastuzumab emtansine (T-DM1) with a viability measurement 96 hours after protein addition.

From the 47 cell lines tested, HER2 surface expression was graded as high (S/I≥100), moderate (SI>10 and <100) or low/negative (S/I≤10). For example, gastric cell line NCI-N87 had high HER2 expression, SNU-216 had moderate HER2 expression, and MKN-45, MKN-1, SNU-1, SCH and Hs 746T lines had low/negative HER2 expression.

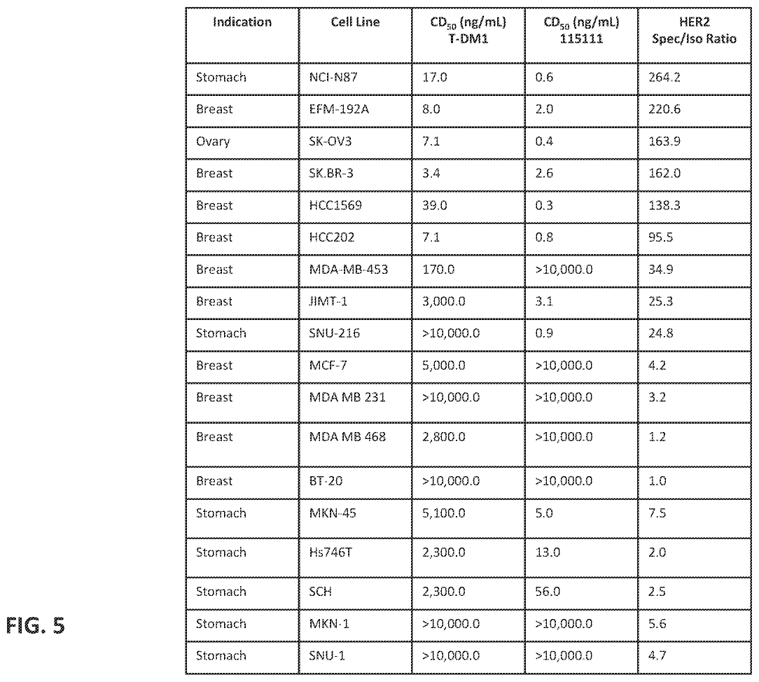

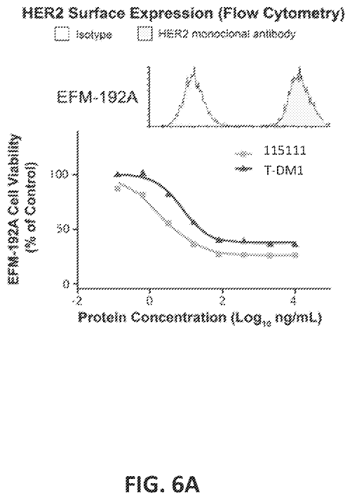

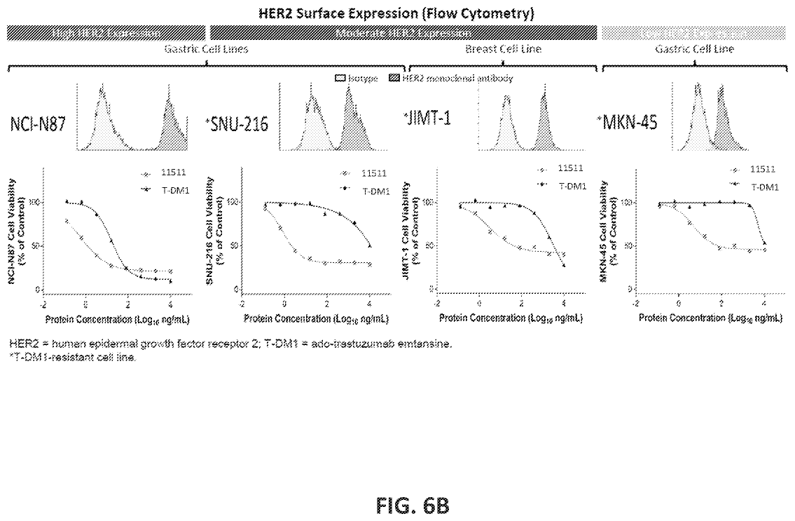

115111 demonstrated potent cytotoxic activity in nearly all of the cell lines with moderate to high HER2 expression (half-maximal cytotoxic concentration CD₅₀ of ˜1-3 ng/mL (FIG. 5)). In cell lines sensitive to 115111, the activity is similar or better than T-DM1. In HER2-positive cell lines, with the exception of the MDA-MB-453 cell line, the IC40 was >500-fold lower as compared to HER2-negative cell lines. T-DM1-resistant cell lines with moderate cell surface HER2 expression (JIMT-1 breast cancer, SNU-216 gastric cancer) were sensitive to 115111 but were not effectively killed by T-DM1 (FIG. 6A-6B).

No cytotoxicity was observed on multiple HER2-negative cell lines.

### Example 3: 115111 Kills HER2-Positive HCC1954 Cells at Concentrations Below Levels Needed for Receptor Saturation

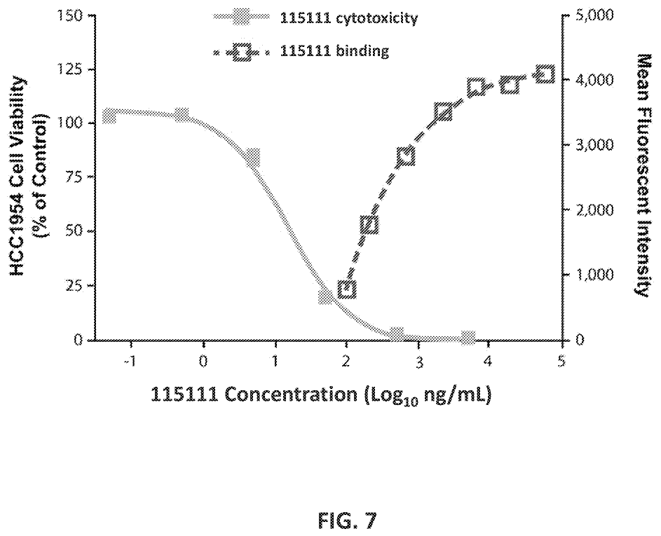

Binding of 115111 to HER2-positive cells was evaluated. Briefly, 115111 was added to HCC1954 cells and the cells were incubated for 1 hour on ice. Binding was measured using a flow-based assay using a labeled anti-toxin monoclonal antibody for detection and reported as mean fluorescence intensity as a function of protein concentration. Saturation of 115111 binding to HER2 on the cell surface was observed at concentrations >6,700 ng/mL (FIG. 7, right axis).

Cytotoxicity of 115111 was measured 96 hours after addition to a high-density assay format of HCC1954 cells using Cell Titer-Glo® (Promega®). 115111 kills target cells in this assay format with a half-maximal cytotoxic concentration (CD₅₀) of 15 ng/mL, with 80% killing observed at 50 ng/mL (FIG. 7, left axis).

### Example 4: 115111 Effectively Kills Trastuzumab-Resistant Cells and Demonstrates Cytotoxicity in the Presence of Approved HER2-Targeted Antibodies In Vitro

HER2-positive HCC1954 cells have been reported to be insensitive to trastuzumab. HCC1954 cells (low density format) were either pre-treated with vehicle or one or two HER2-targeted monoclonal antibodies (100 μg/mL each) for 1 hour prior to addition of 115111. The cytotoxic activity of 115111 on these cells was measured by Cell Titer-Glo® (Promega®) 120 hours after protein addition.

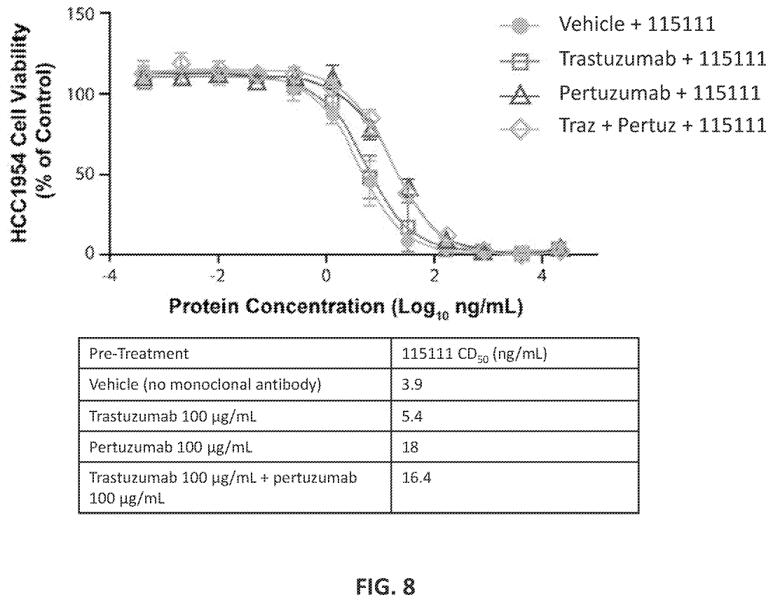

As shown in FIG. 8, 115111 had potent activity on the HCC1954 cells. Cytotoxicity of 115111 on HCC1954 cells was minimally affected (IC₅₀ within 5-fold of a control) in the presence of either traztuzumab or pertuzumab, or in the presence of both in combination.

### Example 5: 115111 Specifically Binds to Both Human and Non-Human Primate HER2 Protein

An enzyme-linked immunosorbent assay (ELISA) using recombinant HER2 protein from human and cynomolgus monkey sequences and an anti-toxin monoclonal antibody was used to determine the cross-species reactivity of 115111.

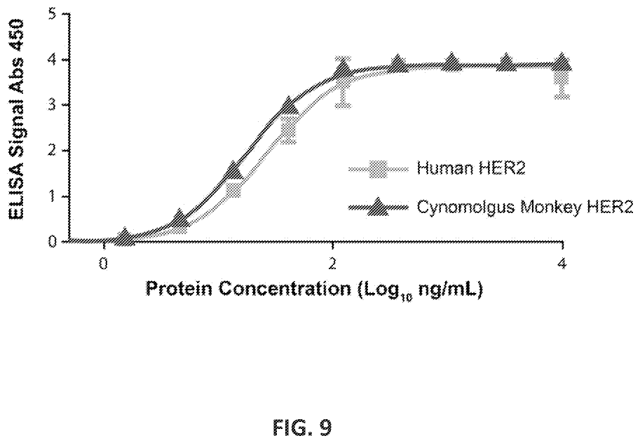

The K_{D} was measured to be 26 ng/mL for human HER2 and 18 ng/mL for cynomolgus monkey HER2 (FIG. 9). Because 115111 binds to cynomolgus monkey and human HER2 protein with similar affinity, the cynomolgus monkey is a relevant model for toxicology studies.

### Example 6: 115111 Good Laboratory Practice Studies in Non-Human Primates Indicate Planned First-In-Human Doses Will Achieve 115111 Exposure Above Levels Needed for In Vitro Cellular Cytotoxicity of HER2-Positive Tumor Cells

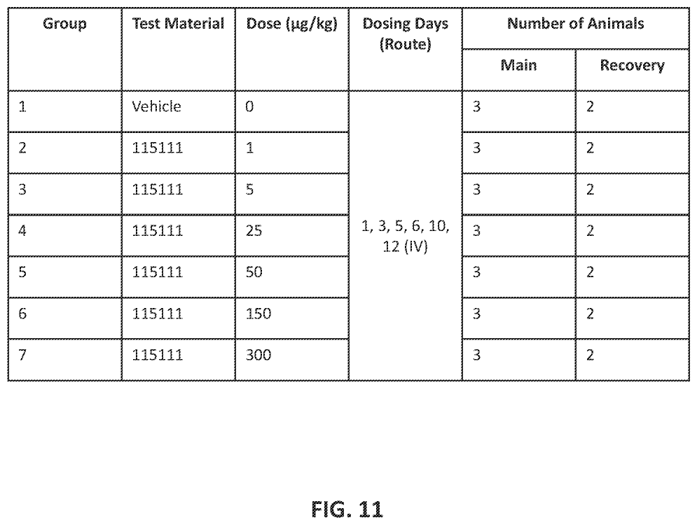

Good Laboratory Practice toxicology studies of 115111 in non-human primates (NHPs) were performed as outlined in FIG. 11. As explained above, the cynomolgus monkey is a pharmacologically relevant species to evaluate toxicity of 115111. 115111 was administered at a more frequent dosing schedule (3×/week) in the NHP toxicity study than the phase 1 study (weekly). Dose-dependent toxicity observed in primates included: increased circulating troponin-1 levels at ≥25 μg/kg (minimal at 25 μg/kg); increased ECG findings (atrioventricular block) at ≤50 μg/kg; increased myocardial degeneration/damage at ≥150 μg/kg. The highest non-severely toxic dose (HNSTD) was 5 μg/kg.

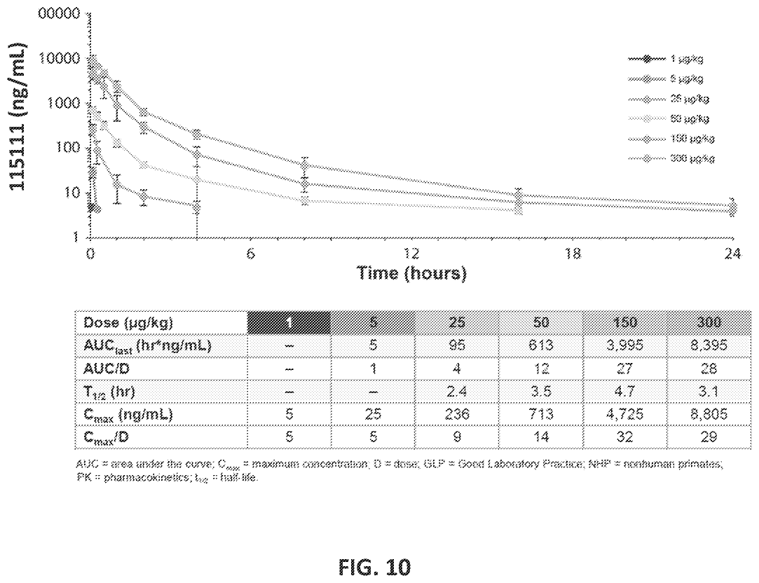

Pharmacokinetic (PK) data was measured after the first intravenous dose using a Meso Scale Discovery-based assay and is shown in FIG. 10. Based on dose-normalized area under the curve and maximum concentration values, less than dose-proportional PK was observed at doses ≤150 μg/kg. The 115111 half-life in NHP was approximately 2 to 5 hours.

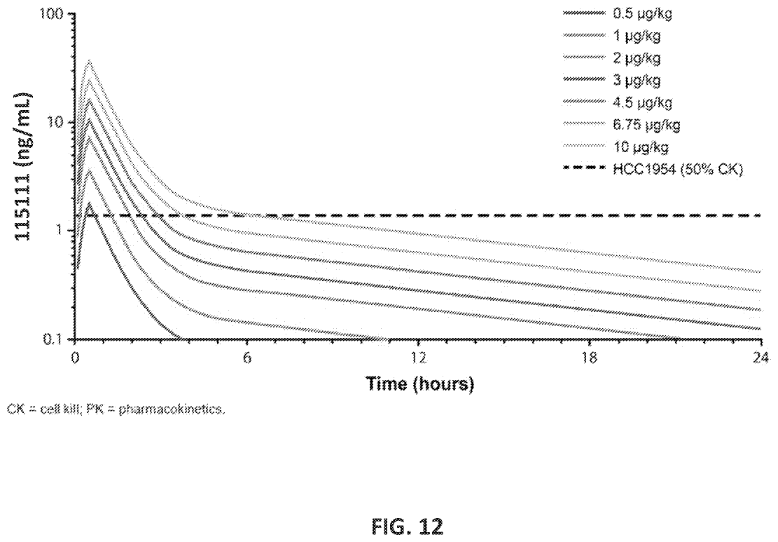

The simulated human PK using the Dedrick model is shown in FIG. 12. Simulations were based on the 25 μg/kg NHP PK data. Post-infusion time above 1.6 ng/mL (mean half-maximal cytotoxic concentration (CD₅₀) on HCC1954 cells from multiple experiments) was calculated to be 0.1 to 4.8 hours (˜0.1 hours (0.5 μg/kg), ˜0.9 hours (1 μg/kg), ˜1.6 hours (2 μg/kg), ˜2 hours (3 μg/kg), ˜2.5 hours (4.5 μg/kg), ˜3.2 hours (6.75 μg/kg), ˜4.8 hours (10 μg/kg)). This modeling suggests that 115111 can be administered at doses in humans above the in vitro half-maximal cytotoxic concentration (CD₅₀).

### Example 7: Phase 1, First in Human, Open-Label, Dose Escalation and Expansion Clinical Study of 115111 in Subjects with HER2-Positive Solid Tumors

115111 will be evaluated as a monotherapy in a first-in human, open-label study in subjects with HER2-positive locally advanced or metastatic solid cancers. This study will be conducted in two sequential parts: Part A and Part B (See FIGS. 13A, 13B, and 13C). The purpose of Part A is to determine the Recommended Phase 2 dose (RP2D) to be used in Part B. Part A will include any type of HER2-positive solid cancer. The purpose of Part B is to confirm the safety and tolerability of the RP2D of 115111. Part B will include three planned cohorts: breast cancer, gastric or gastroesophageal adenocarcinomas (GEA); and any other type of HER2-positive solid cancer.

Part A will escalate 115111 doses according to the scheme shown in FIG. 13A to determine the maximum tolerated dose (MTD) or the RP2D. MTD is defined as the highest 115111 dose that can be given so that no more than 33% of evaluable subjects experiences a dose-limiting toxicity (DLT). Therefore, at least 3 evaluable subjects must be treated with 115111 at this dose level before the MTD can be confirmed (3+3 design). If no DLT occurs at any of the dose levels tested, an RP2D of 115111 will be determined based on all available PK and PD data.

Eligible subjects will be identified and treated through competitive enrollment at multiple study centers. In Parts A and B of the study, a subject may participate for the following four periods: screening (up to 28 days before first dose of 115111); treatment period (active period where a subject will receive doses of 115111 over a 21-day treatment cycle); follow-up (30 days after last dose of 115111); long-term follow-up (every 3 months for up to 24 months after the last dose of 115111).

115111 will be given as an intravenous (IV) infusion over about 30 minutes (+10 minutes) on the same day every week (i.e., on day 1, day 8 and day 15 of each cycle, a cycle being defined as 21 days, with a permissible window of +/−2 days around each weekly infusion). A subject can continue receiving 115111 as long as it is well-tolerated, their disease has not worsened, or until the subject decides that they no longer want to participate in the study. Dose escalation for Part A of the study is shown in FIG. 13A. Subjects in Cohort 1-7 will be treated with 0.5 μg/kg, 1 μg/kg, 2 μg/kg, 3 μg/kg, 4.5 μg/kg, 6.75 μg/kg, or 10 μg/kg. If <33% of evaluable subjects have a DLT, dose escalation will continue in Part A following Cohort 7. Dose escalation of 115111 following Cohort 7 will be a 25-50% increase from the previous Cohort. For example, 115111 doses in Cohort 8 will range from 12.5-15 μg/kg, 115111 doses in Cohort 9 will range from 15.63-22.5 μg/kg, and 115111 doses in Cohort 10 will range from 19.54-33.75 μg/kg. Additional dose escalation beyond Cohort 10 is possible, provided treatment with 115111 remains tolerable.

If 10 μg/kg is tolerable in Part A, the starting dose of 115111 for Group B1 will be 10 μg/kg for the first 6 subjects, otherwise the starting dose will be lower than 10 μg/kg for the first 6 subjects. Dose escalation will be permitted in Group B1 based on the absence or presence of DLTs in these initial 6 subjects. Only doses that have been proven tolerable in Part A will be considered as options for dose escalation in Group B1.

Enrollment in Group B2 and Group B3 will start after the MTD or RP2D of is determined in Part A. The starting dose of 115111 in Group B2 and Group B3 will be the MTD or RP2D determined in Part A.

Evaluation of safety of 115111 will be measured by number of subjects with adverse events using Common Terminology Criteria for Adverse Events (CTCAE) v 5.0. Evaluation of tolerability of 115111 will be measured by number of subjects with dose limiting toxicities (DLTs). Pharmacodynamic assessments include expression of HER2, estrogen receptor, progesterone receptor, and Ki67 on the tumor cell at screening, as well as serum HER2 throughout the study. Immunogenicity of 115111 will also be evaluated in subjects by assessing anti-drug antibodies (ADA) and neutralizing antibodies. Pharmacokinetics will also be evaluated, by measurement of free 115111 (e.g., maximum plasma concentration (Cₘₐₓ), time to reach maximum concentration after drug administration (Tₘₐₓ), area under the curve). Tumor response will also be evaluated. The objective response rate (ORR) will be defined as the proportion of subjects with either a complete response or a partial response as determined by investigator assessment. Immunogenicity will be measured by anti-drug antibody and neutralizing antibody titer.

Outcome measures will be correlated with the expression of HER2 on the tumor cell analyzed by immunohistochemistry, and with serum-HER2 levels (s-HER2).

Adults 18 years or older are eligible for the study if they have a histologically confirmed, unresectable, locally advanced or metastatic solid cancer that is HER2-positive and the malignancy is relapsed, refractory to, or intolerant of existing therapy(ies). Additional inclusion criteria the study include:

1. Histologically confirmed, unresectable, locally advanced or metastatic solid cancers. In Part A (Dose-Escalation), all HER2-positive solid cancers are eligible. In Part B (Dose-Expansion), any type of HER2-positive solid cancer, including breast cancer, gastric cancer, or gastroesophageal adenocarcinomas (GEA) is eligible.

2. HER2-positive in the latest tumor sample tested for HER2 (testing to be done on a metastatic lesion in cases of metastatic cancers). Tumors tested by immunohistochemistry (IHC) must have an IHC status of 2+ or 3+ regardless of in situ hybridization (ISH) results. For breast and gastric cancers, if no IHC is available, ISH evidence of HER2 amplification per ASCO-CAP guidelines will be accepted.

3. Relapsed or refractory to or intolerant of existing therapy(ies) known to provide clinical benefit for the underlying cancer, or intolerant of such therapies. Subjects with HER2-positive breast cancer should have received at least two lines of HER2-directed therapy in the advanced setting and should have received pertuzumab trastuzumab emtansine, tucatinib, or fam-trastuzumab deruxtecan in either the early-stage or advanced setting. Subjects with HER2-positive gastric cancer must have previously received trastuzumab or fam-trastuzumab deruxtecan or have been intolerant of such therapy. Subjects with tumors that are HER2 2+ by IHC and without gene amplification are not required to have received prior HER2-targeting therapy.

4. At least 1 measurable or evaluable lesion according to RECIST 1.1.

5. ECOG performance score of ≤1.

6. Adequate bone marrow function: Absolute neutrophil count (ANC)≥1,000/mm³, platelet count≥75,000 mms and Hemoglobin≥8.0 g/dL. No red blood cell transfusion within 4 weeks of study treatment start is allowed.

7. Adequate kidney function: estimated glomerular filtration rate (eGFR)≥50 mL/min calculated by the Cockcroft-Gault formula, subjects with CLcr≥50 mL/min will be eligible irrespective of the eGFR result.

8. Adequate cardiac function: Left ventricular ejection fraction (LVEF)≥55% on the multigated acquisition (MUGA) scan (preferred) or echocardiogram (ECHO) assessment, and QTcF≤480 ms for women and QTcF≤450 ms for men [average from three QTcF values on the triplicate 12-lead electrocardiogram (ECG)] at baseline.

9. Adequate hepatic function: Total bilirubin≤1.5×ULN, and AST≤3×ULN and ALT≤3×ULN (<5×ULN (if hepatic metastases)).

10. A life expectancy of at least 3 months.

11. Adequate serum albumin: albumin≥2.5 g/dL.

12. Adequate coagulation: international normalized ratio or prothrombin time≤1.5×ULN, and partial thromboplastin time≥1.5×ULN.

14. Women of reproductive potential must have a negative pregnancy test.

Exclusion Criteria for the Study Include:

1. History or current evidence of another tumor that is histologically distinct from the tumor under study.

2. Current evidence of new or growing CNS metastases during screening. Subjects with known CNS metastases will be eligible if they meet specified criteria.

3. Evidence of CTCAE Grade>1 toxicity before the start of treatment, except for hair loss and those Grade 2 toxicities listed as permitted in other eligibility criteria.

4. History or evidence of significant cardiovascular disease.

5. Current evidence of active, uncontrolled hepatitis B virus, hepatitis C virus, human immunodeficiency virus (HIV) (evidenced by detectable viral load by PCR) or acquired immunodeficiency syndrome (AIDS) related illness.

6. Current evidence of ≥grade 2 underlying pulmonary disease.

7. Current evidence of incomplete surgery or radiotherapy at screening, or planned surgery from the start of treatment, except for minor elective surgery approved by study.

8. History or current evidence of significant (CTCAE Grade≥2) infection or wound within 2 weeks before the start of treatment.

9. Known hypersensitivity to the study drug or excipients contained in the study drug formulation.

10. Evidence of hypersensitivity requiring systemic steroids at doses>20 mg/d prednisone equivalent.

11. History of any other medical or psychiatric condition or addictive disorder, or laboratory abnormality that will increase the risks associated with study participation.

12. Women who are pregnant or breastfeeding.

13. Subjects with unintentional weight loss greater than 10% of their body weight over the preceding 3 months or less.

14. Received systemic therapy for the cancer under study or any investigational drug within 4 weeks before the start of treatment.

15. Received therapeutic anticoagulation for a thromboembolic event within 2 weeks before the start of treatment (prophylactic anticoagulation is allowed).

16. Received immunosuppressive agents or other corticosteroids at doses 220 mg prednisone equivalent per day within 2 weeks before the start of treatment.

17. For Part A: received doxorubicin (or another anthracycline) at any time.

18. For Part B: received anthracycline or anthracenedione agents at the doxorubicin equivalent cumulative dose that would increase the risk of cardiomyopathy, or received mitoxantrone at cumulative doses considered to increase the risk of cardiomyopathy.

19. Received granulocyte colony-stimulating factor or granulocyte-macrophage colony-stimulating factor for the treatment of leukopenia within 2 weeks before the start of treatment.

To date, 10 subjects, with a median of 5 prior lines of therapy and a median of 2 prior HER2-targeting regimens, have been treated with 115111 (metastatic cholangiocarcinoma n=5, metastatic breast cancer n=4, metastatic gastro-esophageal junction carcinoma n=1). Thus far, no dose limiting toxicities (DLTs) have been observed in any cohort and 115111 appears to be well tolerated, with no cardiotoxicity to date (cardiotoxicity is a known potential toxicity for HER2 targeted therapies).

The first cohort (0.5 μg/kg/dose) enrolled 4 subjects (metastatic breast cancer, n=2; metastatic cholangiocarcinoma, n=2). Three subjects were female and the mean age was 69 years (median 65, range 64-78). Subjects received a mean of 5 prior lines of therapy (median 4.5, range 3-8). Three subjects completed C1 of treatment without dose-limiting toxicities; 1 subject was inevaluable. Two subjects had progressive disease in C2. A total of 23 adverse events occurred in 4 subjects; all were grade (G) 1-2 except one G3 event of hypertension in a subject with a history of hypertension. There were 2 treatment-related adverse events (G1 chills; G2 aspartate aminotransferase increased in the setting of progressive liver metastases). There was 1 serious, non-treatment-related adverse event (G2 dyspnea) that occurred in the inevaluable subject. No cardiac adverse events were noted, nor clinically significant changes in cardiac biomarkers, an important safety parameter given non-human primate toxicity.

Four subjects are currently being treated in the second (1 μg/kg/dose) and third cohorts (2 μg/kg/dose). No cardiac adverse events or abnormalities in cardiac biomarkers have been noted thus far.

Causally related adverse events reported among to date include the following: grade 1 chills, grade 1 hypophosphatemia, grade 1 nausea, and grade 2 AST elevation in a subject with disease progression in hepatic metastases (all n=1). The ongoing subject from cohort 2 (45 y/o female with metastatic breast cancer) has no evidence of disease progression (the subject only has evaluable disease but no measurable lesions per RECIST 1.1, and is classified as non-complete response, non-progressive disease, similar to table disease) and remains on treatment, now in cycle 5. One subject in cohort 3 with metastatic breast cancer has had a follow-up CT scan at the end of cycle 2 and has stable disease. Six subjects have discontinued for disease progression and two subjects are too early to evaluate.

Taken together, this data indicates that 115111 appears to be well tolerated at the lowest dose with no apparent cardiotoxicity to date. Drug concentrations are expected to be below the level required for in vitro tumor cell killing.

While some embodiments of the invention have been described by way of illustration, it will be apparent that the invention can be put into practice with many modifications, variations and adaptations, and with the use of numerous equivalents or alternative solutions that are within the scope of persons skilled in the art, without departing from the spirit of the invention or exceeding the scope of the claims.

What is claimed is:

1. A method for treating or preventing cancer, the method comprising administering to a subject in need thereof an effective amount of a HER2 binding molecule comprising:
(A) a cytotoxic Shiga toxin A subunit effector polypeptide; and
(B) a binding region capable of specifically binding an extracellular part of human HER2, wherein the binding region comprises:
(a) an immunoglobulin heavy chain variable region comprising: a CDR1 comprising the sequence of SEQ ID NO: 57; a CDR2 comprising the sequence of SEQ ID NO: 58; and a CDR3 comprising the sequence of SEQ ID NO: 59; and
(b) an immunoglobulin light chain variable region comprising: a CDR1 comprising the sequence of SEQ ID NO: 60; a CDR2 comprising the sequence of SEQ ID NO: 61; and a CDR3 comprising the sequence of SEQ ID NO: 62;
wherein the effective amount is a dose in a range of about 0.1 μg/kg to about 50 μg/kg.

2. The method of claim 1, wherein the dose is about 0.5 μg/kg, about 1.0 μg/kg, about 2.0 μg/kg, about 3.0 μg/kg, about 4.5 μg/kg, about 6.75 μg/kg, about 10.0 μg/kg, about 12.5 μg/kg, about 15.0 μg/kg, about 15.6 μg/kg, about 19.5 μg/kg, about 22.5 μg/kg, or about 33.75 μg/kg.

3. The method of claim 1, wherein the dose is in the range of about 12.5 μg/kg to about 15 μg/kg, about 15.6 μg/kg to about 22.5 μg/kg, or about 19.5 μg/kg to about 33.75 μg/kg.

4. The method of any one of claims 1-3, wherein the HER2 binding molecule is administered to the subject by intravenous, subcutaneous, or intramuscular injection.

5. The method of any one of claims 1-3, wherein the HER2 binding molecule is administered to the subject by intravenous injection.

6. The method of claim 5, wherein the HER2 binding molecule is administered to the subject over a period of about 10 minutes to about 1 hour.

7. The method of claim 5, wherein the HER2 binding molecule is administered to the subject over a period of about 30 minutes.

8. The method of any one of claims 1-7, wherein the HER2 binding molecule is administered to the subject once.

9. The method of any one of claims 1-7, wherein the HER2 binding molecule is administered to the subject more than once.

10. The method of claim 9, wherein the HER2 binding molecule is administered to the subject every seven days.

11. The method of claim 9, wherein the HER2 binding molecule is administered to the subject over a 21 day cycle.

12. The method of claim 11, wherein the HER2 binding molecule is administered to the subject on days 1, 8, and 15 of the 21 day cycle.

13. The method of any one of claims 9-12, wherein the subject is administered a dose in the range of about 0.1 μg/kg to about 50 μg/kg at each administration.

14. The method of claim 13, wherein the subject is administered a dose of about 0.5 μg/kg, about 1.0 μg/kg, about 2.0 μg/kg, about 3.0 μg/kg, about 4.5 μg/kg, about 6.75 μg/kg, about 10.0 μg/kg, about 12.5 μg/kg, about 15.0 μg/kg, about 15.6 μg/kg, about 19.5 μg/kg, about 22.5 μg/kg, or about 33.75 μg/kg at each administration.

15. The method of any one of claims 10-14, wherein the method comprises administering to the subject 0.5 μg/kg of the HER2 binding molecule on days 1, 8, and 15.

16. The method of any one of claims 10-14, wherein the method comprises administering to the subject 1.0 μg/kg of the HER2 binding molecule on days 1, 8, and 15.

17. The method of any one of claims 10-14, wherein the method comprises administering to the subject 2.0 μg/kg of the HER2 binding molecule on days 1, 8, and 15.

18. The method of any one of claims 10-14, wherein the method comprises administering to the subject 3.0 μg/kg of the HER2 binding molecule on days 1, 8, and 15.

19. The method of any one of claims 10-14, wherein the method comprises administering to the subject 4.5 μg/kg of the HER2 binding molecule on days 1, 8, and 15.

20. The method of any one of claims 10-14, wherein the method comprises administering to the subject 6.75 μg/kg of the HER2 binding molecule on days 1, 8, and 15.

21. The method of any one of claims 10-14, wherein the method comprises administering to the subject 10.0 μg/kg of the HER2 binding molecule on days 1, 8, and 15.

22. The method of any one of claims 10-14, wherein the method comprises administering to the subject 12.5 μg/kg of the HER2 binding molecule on days 1, 8, and 15.

23. The method of any one of claims 10-14, wherein the method comprises administering to the subject 15.0 μg/kg of the HER2 binding molecule on days 1, 8, and 15.

24. The method of any one of claims 10-14, wherein the method comprises administering to the subject 15.6 μg/kg of the HER2 binding molecule on days 1, 8, and 15.

25. The method of any one of claims 10-14, wherein the method comprises administering to the subject 19.5 μg/kg of the HER2 binding molecule on days 1, 8, and 15.

26. The method of any one of claims 10-14, wherein the method comprises administering to the subject 22.5 μg/kg of the HER2 binding molecule on days 1, 8, and 15.

27. The method of any one of claims 10-14, wherein the method comprises administering to the subject 33.75 μg/kg of the HER2 binding molecule on days 1, 8, and 15.

28. The method of any one of claims 1-27, wherein the method comprises administering to the subject a composition comprising about 0.1 mg/mL to about 1 mg/mL of the HER2 binding molecule.

29. The method of claim 28, wherein the method comprises administering to the subject a composition comprising about 0.5 mg/mL of the HER2 binding molecule.

30. The method of any one of claims 1-27, wherein the method comprises administering to the subject a composition comprising a HER2 binding molecule in a buffer comprising one or more of sodium citrate, sorbitol, and polysorbate 20.

31. The method of claim 30, wherein the buffer has a pH in the range of about 5.3 to about 5.7.

32. The method of claim 31, wherein the buffer has a pH of about 5.5.

33. The method of any one of claims 1-27, wherein the method comprises administering to the subject a composition comprising:
(i) about 0.1 mg/mL to about 1 mg/mL of the HER2 binding molecule;
(ii) about 0.5 mg/mL to about 10 mg/mL sodium citrate;
(iii) about 1 mg/mL to about 100 mg/mL sorbitol; and
(iv) about 0.001% (v/v) to about 0.1% (v/v) polysorbate 20;
wherein the composition has a pH of about 5.3 to about 5.7.

34. The method of any one of claims 1-27, wherein the method comprises administering to the subject a composition comprising:
(i) about 0.5 mg/mL of the HER2 binding molecule;
(ii) about 5.2 mg/mL sodium citrate;
(iii) about 36.4 mg/mL sorbitol; and
(iv) about 0.02% (v/v) polysorbate 20;
wherein the composition has a pH of about 5.5.

35. The method of any one of claims 1-27, wherein the method comprises administering to the subject a composition comprising:
(i) about 0.5 mg/mL of the HER2 binding molecule;
(ii) about 20 mM sodium citrate;
(iii) about 200 mM sorbitol; and
(iv) about 0.02% (v/v) polysorbate 20;
wherein the composition has a pH of about 5.5.

36. The method of any one of claims 1-35, wherein the method comprises administering to the subject a second anti-cancer agent.

37. The method of claim 36, wherein the second anti-cancer agent is a second HER2 binding molecule.

38. The method of claim 37, wherein the second HER2 binding molecule is trastuzumab or pertuzumab.

39. The method of claim 36, wherein the second anti-cancer agent is trastuzumab emtansine, tucatinib, fam-trastuzumab deruxtecan, docetaxel, capecitabine, fluorouracil, or cisplatin.

40. The method of any one of claims 1-39, wherein the cancer is a HER2-positive cancer.

41. The method of claim 40, wherein the cancer is a HER2-positive solid cancer.

42. The method of claim 40, wherein the cancer is an epithelial cancer.

43. The method of claim 40, wherein the cancer is breast cancer, gastric cancer, gastroesophageal adenocarcinoma, cholangiocarcinoma, bladder cancer, gallbladder cancer, testicular cancer, ovarian cancer, uterine cancer, cervical cancer, head and neck cancer, non-small cell lung cancer, or colorectal cancer.

44. The method of claim 43, wherein the cancer is breast cancer, gastric cancer, or gastroesophageal adenocarcinoma.

45. The method of claim 43, wherein the cancer is cholangiocarcinoma.

46. The method of any one of claims 1-45, wherein the cancer is relapsed or refractory to at least one other cancer therapy, or the subject is known to be intolerant of at least one other cancer therapy.

47. The method of any one of claims 1-45, wherein the cancer is relapsed or refractory to at least two prior lines of cancer therapy, or the subject is known to be intolerant of at least two prior lines of cancer therapy.

48. The method of claim 46 or 47, wherein the cancer is relapsed or refractory to trastuzumab, pertuzumab, trastuzumab emtansine, tucatinib, fam-trastuzumab deruxtecan, docetaxel, capecitabine, fluorouracil, cisplatin, or any combination thereof.

49. The method of any one of claims 1-48, wherein the Shiga toxin A Subunit effector polypeptide has the sequence of SEQ ID NO: 20, or a sequence that is at least 95%, at least 96%, at least 97%, at least 98%, or at least 99% identical thereto.

50. The method of any one of claims 1-49, wherein the binding region has the sequence of SEQ ID NO: 224, or a sequence that is at least at least 95%, at least 96%, at least 97%, at least 98%, or at least 99% identical thereto.

51. The method of any one of claims 1-50, wherein the Shiga toxin A subunit effector polypeptide and binding region are fused, forming a continuous polypeptide.

52. The method of any one of claims 1-51, wherein the binding molecule has the sequence of SEQ ID NO: 29, or a sequence that is at least at least 95%, at least 96%, at least 97%, at least 98%, or at least 99% identical thereto.
